(19) United States
(12) Patent Application Publication
(10) Pub. No.: US 2019/0367588 A1
(43) Pub. Date: Dec. 5, 2019

(54) Anti-APOE4 Antigen-Binding Proteins and Methods of Use Thereof

(71) Applicant: Alector LLC, South San Francisco, CA (US)

(72) Inventor: Arnon Rosenthal, Woodside, CA (US)

(73) Assignee: Alector LLC, South San Francisco, CA (US)

(21) Appl. No.: 16/244,204

(22) Filed: Jan. 10, 2019

Related U.S. Application Data
(62) Division of application No. 15/414,955, filed on Jan. 25, 2017, now abandoned.
(60) Provisional application No. 62/288,196, filed on Jan. 28, 2016.

(51) Int. Cl. C07K 16/18; A61K 45/06

(52) U.S. Cl. CPC C07K 16/18; A61K 2039/505; A61K 45/06

(57) ABSTRACT

The present disclosure generally relates to antigen-binding proteins (ABPs) that specifically bind to apolipoprotein E (ApoE), compositions comprising such ABPs, methods of using such ABPs, and methods of making such ABPs. In some embodiments, the ABPs provided herein bind lipidated ApoE4. In some embodiments, the lipidated ApoE4 is within a lipoprotein particle, and the ABPs therefore bind to a lipoprotein particle comprising ApoE4. Any suitable ABP may be used. In some embodiments, the ABP is an antibody. In some embodiments, the ABP is an alternative scaffold. The ABPs provided herein may be used for the prevention or treatment of any disease, condition or disorder associated with ApoE4 expression.

This application is a divisional of U.S. application Ser. No. 15/414,955, filed Jan. 25, 2017, which claims the benefit of U.S. Provisional Application No. 62/288,196, filed Jan. 28, 2016, each of which is hereby incorporated by reference in its entirety.

### SEQUENCE LISTING

This application contains a Sequence Listing which has been submitted electronically as a text file via EFS-Web and is hereby incorporated by reference in its entirety. The text file, created Jan. 3, 2017, is named ApoE4_Sequence_Listing_ST25.txt and is 6 KB in size.

### FIELD

The present disclosure generally relates to antigen-binding proteins (ABPs) that specifically bind to apolipoprotein E (ApoE), compositions comprising such ABPs, methods of using such ABPs, and methods of making such ABPs. In some embodiments, the ABPs provided herein bind lipidated ApoE4. In some embodiments, the lipidated ApoE4 is within a lipoprotein particle, and the ABPs therefore bind to a lipoprotein particle comprising ApoE4. Any suitable ABP may be used. In some embodiments, the ABP is an antibody. In some embodiments, the ABP is an alternative scaffold. The ABPs provided herein may be used for the prevention or treatment of any disease, condition or disorder associated with ApoE4 expression, such as dementia, cognitive disorder, Alzheimer's disease, cerebral amyloid angiopathy, traumatic brain injury, stroke, epilepsy, multiple sclerosis, age-related macular degeneration.

### BACKGROUND

Apolipoprotein E (ApoE) is a glycoprotein of 299 amino acids, with a molecular mass of 34 kDa. It is synthesized with sialic acid attached by O-glycosidic linkage and is subsequently desialylated in plasma. O-glycosylation is with core 1, or possibly core 8, glycans. Thr-307 and Thr-314 are minor glycosylation sites, while Ser-308 is a major glycosylation site. ApoE is glycated in plasma VLDL of normal subjects, and glycated at a higher level (2-3 fold) in plasma of hyperglycemic diabetic patients.

ApoE is genetically polymorphic, exhibiting multiple isoforms as detected by isoelectric focusing. The polymorphism is the result of three alleles at a single gene locus designated ApoE4, ApoE3, and ApoE2, with ApoE4 being the most cationic and differing from ApoE3 by one charge unit and from ApoE2 by two charge units. The corresponding alleles for the three isoforms were termed ε4, ε3, and ε2 and differ in their frequencies: ε4 (15-20%), ε3 (65-70%), and ε2 (5-10%). They give rise to six phenotypes, all of which are readily detectable in human subjects: three homozygous phenotypes (ε4/4, ε3/3, and ε2/2) and three heterozygous phenotypes (ε4/3, ε3/2, and ε4/2). Genetic ApoE isoforms differ at two sites: ApoE3 has cysteine at position 112 and Arg at position 158, ApoE4 has arginines at both positions, and ApoE2 has cysteines at both positions.

The gene coding for the three isoforms of ApoE resides on the long arm of chromosome 19 in humans. The gene has 3.6 kilobases with three introns and codes for a 317 residue precursor protein; an 18 amino acid prepeptide that signals secretion is cotranslationally removed. ApoE is synthesized and secreted by many tissues, primarily liver, brain, skin, and tissue macrophages throughout the body. Plasma ApoE (40-70 mg/ml) arises primarily from hepatic synthesis (75%). The second most common site of synthesis is the brain. Astrocytes produce a large proportion of cerebrospinal fluid ApoE (3-5 mg/ml), while neurons synthesize ApoE when stressed. ApoE, is a main lipid-binding protein of the lipoproteins, which include chylomicrons, lipoprotein particles that consist of triglycerides (85-92%), phospholipids (6-12%), cholesterol (1-3%) and proteins (1-2%), very low density lipoproteins (VLDL), intermediate density lipoproteins (IDL), and a subclass of high density lipoproteins (HDL). ApoE proteins are present on lipoprotein in association with other apolipoproteins. In the brain, ApoE is associated with two other apolipoproteins, ApoJ and ApoA-1, predominantly on high-density-like lipoprotein particles. Unlike plasma HDL that contains ApoA-1 as its major apolipoprotein, the predominant apolipoprotein of HDL in the central nervous system (CNS) is ApoE. Although HDL-like lipoproteins are the only lipoproteins in the central nervous system (CNS), their role in CNS lipid and cholesterol homeostasis is not clearly defined.

The five major groups of lipoproteins (chylomicrons, VLDL, IDL, low density lipoproteins (LDL), and HDL) enable fats and cholesterol to move within the water-based solution of the bloodstream. They transport exogenous lipids to liver, adipose, cardiac, and skeletal muscle tissue, where their triglyceride components are unloaded by the activity of lipoprotein lipase. The contents of lipoproteins taken into a cell are stored, used for cell membrane structure, or converted into other products such as steroid hormones or bile acids.

Structural analyses provides insight into the mechanisms of ApoE's involvement in cardiovascular, neurological, and infectious diseases. ApoE has two structural domains separated by a hinge region. The N-terminal domain (amino acids 1-191) contains the receptor-binding region (amino acids 134-150 and Arg-172) and forms a four-helix antiparallel bundle. The C-terminal domain (amino acids 225-299) contains the major lipid-binding region centered on amino acids 244-272. The amino acid differences among the isoforms profoundly affect their structures when in complexes with lipids and roles in disease. For example, there are interactions among amino acids 154-158 in ApoE3 and ApoE4. Such interactions are not present in ApoE2, and therefore amino acid 154 of ApoE2 interacts by salt bridge with amino acid 150. This disrupts the ability of ApoE2 to bind the LDLR. (J Lipid Res (2000) 41: 1087-1095; J Lipid Res (1998) 39: 1173-1180).

ApoE4 appears to increase the concentrations of atherogenic lipoproteins and to accelerate atherogenesis. Understanding structural differences in ApoE isoforms helps elucidate molecular mechanisms responsible for the associated pathology. The increase in plasma cholesterol, LDL, and in the lipoprotein ApoB that are associated with the ApoE4 allele appear to reflect the influence of Arg-112. This amino acid alters the lipid-binding region of ApoE4 and changes its lipid binding preference from small phospholipid-rich HDL to large triglyceride-rich VLDL. This preference is specific to ApoE4 and is not displayed by ApoE2 or ApoE3 (Biochemistry (2010) 49:10881-10889; Biochemistry (2008) 47:2968-2977). This difference is due to ApoE4 domain interactions, in which the N- and C-terminal domains interact, resulting in a more compact structure.

In addition to being critical for lipid binding, the basic amino acid residues arginine and lysine were shown to be critical for high affinity binding of ApoE2 and ApoE3 to the LDL receptor. Mutagenesis studies identified critical basic residues required for receptor binding within the residue 134-150 region of ApoE, as well as Arg-172. Modeling of ApoE bound to phospholipid revealed why lipid binding is required for high-affinity binding to LDL receptors. To fit the molecular envelope of phospholipids, ApoE folded into a helical horseshoe, bringing critical residues for receptor binding, amino acids 134-150 and Arg-172, into close proximity. When complexed with lipids, this region is largely exposed to solvent and forms a 20 Å field of positive potential, which is available for receptor binding. In contrast, in ApoE2, the presence of Cys-158 disrupts a salt bridge, which, in ApoE3 and ApoE4, forms between Arg-158 and Asp-154. This alters the size of the positively charged domain leading to ApoE2 being defective in LDL receptor binding activity (2% LDL receptor binding activity compared with ApoE3 or ApoE4). Nevertheless, ApoE2 can still mediate lipoprotein clearance through binding heparin sulfate proteoglycans (HSPGs).

The C-terminal domain of ApoE (amino acids 225-299 and more specifically residues 261-272) is predicted to rearrange and form amphipathic α-helices, which are responsible for lipid binding primarily at amino acids 244-272. ApoE3 and ApoE2 preferentially bind to small, phospholipid-rich HDL, whereas ApoE4 binds to large, triglyceride-rich VLDL. The preferential binding of ApoE4 to VLDL is a result of its high lipid binding ability coupled with the fact that ˜60% of the VLDL particle surface is covered with phospholipids. In contrast, the surface of HDL particles is 80% covered with apolipoproteins and ApoE-lipid interactions are less important for binding to HDL. Instead, binding to HDL is mediated largely through interactions between the N-terminal helix bundle domains of ApoE2 and ApoE3 with the resident apolipoproteins on HDL (Biochemistry. (2010); 49: 10881-10889).

The high lipid binding ability of ApoE4 is caused by allele specific orientation of the side chain and rearrangements of salt bridges, which allows interactions between the C and N terminal regions of ApoE4. Specifically, in ApoE4, Arg-112 forms a salt bridge with Glu-109 and causes the Arg-61 side chain to extend away from the four-helix bundle. In ApoE3, this side chain is buried. The orientation of Arg-61 in ApoE4 promotes interaction with Glu-255, within the lipid-binding region, causing ApoE4 to have a more compact conformation than ApoE3.

ApoE4 exhibits greater lipid binding ability than ApoE3 as a consequence of a rearrangement involving the segment spanning residues 261-272 in the C-terminal domain. The high lipid binding ability of ApoE4 coupled with the VLDL particle surface being ˜60% phospholipid (PL) covered is the basis for its preference to bind to VLDL rather than HDL. ApoE4 binds much more than ApoE3 to VLDL.

As discussed above, domain interaction is an important structural property of ApoE4 that may be responsible for some of its pathogenic effects. Mutation of Arg-61 to threonine, or Glu-255 to alanine, abolishes domain interaction, causing the mutated ApoE4 to function similarly to ApoE3 with regard to lipid preference. Because ApoE4 binds preferentially to VLDL and chylomicron remnants, it may accelerate clearance through the LDLR and therefore lead to down regulation of the LDLR and to a pathological increase in LDL levels.

Histopathological and imaging studies revealed a positive correlation between amyloid plaque density, fibrillar amyloid beta burden and the number of ApoE4 alleles. Likewise, the level of soluble amyloid beta in the cerebrospinal fluid (CSF) was found to be lower in ApoE4 carriers, indicating that deposition of soluble amyloid beta in amyloid plaques, and its depletion from the CSF, begins earlier in ApoE4-positive subjects. All ApoE isoforms were shown to bind amyloid beta. However, lipid-associated ApoE2 and ApoE3 form SDS-stable complexes with amyloid beta to a much greater extent than ApoE4, and efficiency of complex formation between lipidated ApoE and amyloid beta follows the order of ApoE2>ApoE3>>ApoE4. Since the binding efficiency of ApoE isoforms to amyloid beta correlates inversely with the risk of developing AD, it has been hypothesized that ApoE4 is unable to clear amyloid beta. However, it remains possible that ApoE4 facilitates pathological aggregation of amyloid beta (J Neurosci (2013) 33:358-370).

Inflammation and abnormal activation of astrocytes and microglia are common pathological features of Alzheimer's disease (AD), along with amyloid plaques and neurofibrillary tangles. Activated glial cells are closely associated with amyloid plaques, suggesting that plaques or soluble forms of amyloid beta around plaques may induce inflammatory cascades. Consistent with neuropathological findings, amyloid beta was shown to trigger glial neuroinflammatory responses in cell culture systems. Interestingly, amyloid beta induces the production of ApoE and the increased levels of ApoE limit Aβ-driven neuroinflammation, implying that ApoE may have general anti-inflammatory effects. Consistent with the observed anti-inflammatory role of ApoE in vitro, lack of ApoE expression in mice was associated with increased inflammation, including induction of several cytokines and proinflammatory responses, in response to treatment of amyloid beta and other activating stimuli. Several studies demonstrated that exogenously applied ApoE4 has weak anti-inflammatory activity and in fact displays robust proinflammatory activity on astrocytes and microglial cells. Likewise, ApoE4 knockin mice display greater inflammatory responses to intravenous administration of LPS, compared with ApoE3 knockin mice. Thus, ApoE4 may have proinflammatory or less effective anti-inflammatory function and therefore may exacerbate detrimental neuroinflammation in AD.

In addition to its role the aggregation and clearance of amyloid beta, ApoE4 my affect AD onset and progression by modulating the function of the cerebrovascular system and brain metabolism. The ApoE4 isoform has been linked to increased levels of LDL and has been shown to be a risk factor for cardiovascular disease. As a result, increased levels of atherosclerosis associated with ApoE4 could have detrimental effects on brain function through decreased blood flow and altered metabolic properties. Positron emission tomography (PET) studies have shown that AD brains exhibit decreased glucose metabolism in distinct regions. Furthermore, studies looking at both young and old non-demented carriers of the ApoE4 isoform observed a similar regional pattern of hypometabolism prior to the onset of disease that correlates with the changes seen in the AD brain.

Finally, ApoE4 has been associated with leakage in the blood-brain barrier (BBB) (Molecular Medicine (2001) 7(12):810-815; J. Biol. Chem. (2011) 286:17536-17542). Specifically, in vitro BBB models consisting of brain endothelial cells and pericytes prepared from wild-type (WT) mice, and primary astrocytes prepared from human ApoE3- and ApoE4-knock-in mice, revealed that the barrier function of tight junctions (TJs) was impaired, and the phosphorylation of the tight junction protein occludin at threonine residues and the activation of protein kinase C were attenuated when the BBB was reconstituted with primary astrocytes from ApoE4-knock-in mice. Consistent with the results of in vitro studies, BBB permeability was higher in ApoE4-knock-in mice than in ApoE3-knock-in mice. Thus, ApoE4-knock-in mice display BBB breakdown and activation of the proinflammatory CypA-nuclear factor-κB-matrix-metalloproteinase-9 pathway in pericytes. This, in turn, leads to neuronal uptake of multiple blood-derived neurotoxic proteins, and microvascular and cerebral blood flow reductions. In addition, ApoE4 was associated with disrupted perivascular drainage of soluble amyloid beta from the brain. This effect may be mediated, in part, by changes in age-related expression of basement membrane proteins in the cerebral vasculature. The vascular defects in ApoE4-expressing mice precede neuronal dysfunction and can initiate neurodegenerative changes (Nature (2012) doi:10.1038/nature11087; PLoS ONE (2012) 7(7):e41636. doi:10.1371/journal.pone.0041636).

All references cited herein, including patent applications and publications, are hereby incorporated by reference in their entirety.

### SUMMARY

The present disclosure is generally directed to ABPs that specifically bind to ApoE4 (“ApoE4 ABPs”). In some embodiments, the ABPs specifically bind to lipidated ApoE4. In some embodiments, the ABPs provided herein preferentially bind lipidated ApoE4 as compared to unlipidated ApoE4.

In some embodiments, the lipidated ApoE4 is associated with a lipoprotein particle. In some aspects, the lipoprotein particle is selected from a chylomicron, an HDL particle, an IDL particle, an LDL particle, a VLDL particle, and combinations thereof. In some embodiments, the lipoprotein particle further comprises at least one lipoprotein other than ApoE4.

In some embodiments, the ApoE4 ABPs provided herein are isolated antibodies. In some aspects, the antibodies are selected from monoclonal antibodies, human antibodies, humanized antibodies, chimeric antibodies, bispecific antibodies, and antibody fragments. In some embodiments, the ABPs provided herein are alternative scaffolds, as described in more detail elsewhere in this disclosure.

In some embodiments, an ApoE4 ABP provided herein binds lipidated ApoE4 with an affinity greater than (as indicated by lower K_{d}) the affinity of the ABP for non-lipidated ApoE4. In some aspects, the affinity of the ABP for lipidated ApoE4 is at least 2-fold, at least 3-fold, at least 4-fold, at least 5-fold, at least 10-fold, at least 20-fold, at least 50-fold, at least 100-fold, at least 200-fold, at least 500-fold, at least 1000-fold, or at least 10,000-fold greater than the affinity of the ABP for non-lipidated ApoE4.

In some embodiments, an ApoE4 ABP provided herein binds lipidated ApoE4 with an affinity greater than (as indicated by lower K_{d}) the affinity of the ABP for ApoE2 and/or ApoE3. In some aspects, the affinity of the ABP for lipidated ApoE4 is at least 2-fold, at least 3-fold, at least 4-fold, at least 5-fold, at least 10-fold, at least 20-fold, at least 50-fold, at least 100-fold, at least 200-fold, at least 500-fold, at least 1000-fold, or at least 10,000-fold greater than the affinity of the ABP for ApoE2 and/or ApoE3.

In some embodiments, an ApoE4 ABP provided herein binds lipidated ApoE4 with an affinity greater than (as indicated by lower K_{d}) the affinity of the ABP for lipidated ApoE2 and/or lipidated ApoE3. In some aspects, the affinity of the ABP for lipidated ApoE4 is at least 2-fold, at least 3-fold, at least 4-fold, at least 5-fold, at least 10-fold, at least 20-fold, at least 50-fold, at least 100-fold, at least 200-fold, at least 500-fold, at least 1000-fold, or at least 10,000-fold greater than the affinity of the ABP for lipidated ApoE2 and/or lipidated ApoE3.

In some embodiments, an ApoE4 ABP provided herein binds lipidated ApoE4 with an affinity (as measured by K_{d}) of 10⁻⁶ M or less, 10⁻⁷ M or less, 10⁻⁸ M or less, 10⁻⁹ M or less, 10⁻¹⁰ M or less, or 10⁻¹¹M or less. In some aspects, the lipidated ApoE4 is associated with a lipoprotein particle.

In some embodiments, the ApoE4 protein is a mammalian protein. In some aspects, the mammalian protein is a human protein. In some aspects, the ApoE4 protein is a wild-type protein. In some aspects, the ApoE4 protein is a naturally occurring variant. In some aspects, the ApoE4 protein is a glycated or glycosylated ApoE4 protein.

In some embodiments, the ApoE4 ABP binds to one or more amino acid residues within amino acid residues selected from: (a) amino acid residues 55-78 (QVTQELRALMDETMKELKAYKSEL (i.e., SEQ ID NO: 2)) of SEQ ID NO: 1; (b) amino acid residues 134-150 (RVRLASHLRKLRKRLLR (i.e., SEQ ID NO: 3)) of SEQ ID NO: 1; (c) amino acid residues 154-158 (DLQKR (i.e., SEQ ID NO: 4)) of SEQ ID NO: 1; (d) amino acid residues 208-272 (QAWGERLRARMEEMGSRTRDRLDEVKEQVAEVRAKLEEQAQQIRLQAEAFQARLKSWFEPLV EDM (i.e., SEQ ID NO: 5)) of SEQ ID NO: 1; (e) amino acid residues 225-299 (TRDRLDEVKEQVAEVRAKLEEQAQQIRLQAEAFQARLKSWFEPLVEDMQRQWAGLVEKVQA AVGTSAAPVPSDNH (i.e., SEQ ID NO: 6)) of SEQ ID NO: 1; and (f) amino acid residues 244-272 (EEQAQQIRLQAEAFQARLKSWFEPLVEDM (i.e., SEQ ID NO: 7)) of SEQ ID NO: 1. In some embodiments, the ABP binds to an epitope of SEQ ID NO: 1 comprising at least one of amino acid residues Arg-61, Glu-109, Arg-112, Arg-136, His-140, Lys-143, Arg-150, Asp-154, Arg-158, Arg-172 and Glu-255.

In some embodiments, the ApoE4 ABP disrupts the interaction between an N-terminal domain and C-terminal domain of an ApoE4 protein. In certain embodiments, the ABP disrupts the interaction between helix 2 comprising amino acid residues 55-78 (QVTQELRALMDETMKELKAYKSEL (i.e., SEQ ID NO: 2)) of SEQ ID NO: 1 and the lipid binding domain comprising amino acid residues 244-272 (EEQAQQIRLQAEAFQARLKSWFEPLVEDM (i.e., SEQ ID NO: 7)) of SEQ ID NO: 1. In certain embodiments, the ABP disrupts the interaction between amino acid residues Arg-61 and Glu-255 of SEQ ID NO: 1.

In some embodiments, the ApoE4 ABP modulates a function of, or phenotype associated with, ApoE4. In some embodiments, the function of, or phenotype associated with, ApoE4 is selected from one or more functions or phenotypes provided in Table 1. In some embodiments, the ABP modulates the respective function or phenotype so that such function or phenotype more closely resembles the corresponding function or phenotype of ApoE2.

Also provided are isolated nucleic acid molecules encoding the ABPs provided herein, or portions (e.g., antigen-binding fragments) thereof. In some aspects, provided herein are isolated nucleic acid molecules comprising nucleotide sequences that encode the heavy chain and/or light chain variable region of an anti-ApoE4 antibody described herein. In some aspects, provided herein are isolated nucleic acid molecules comprising nucleotide sequences that encode the heavy chain and/or light chain of an anti-ApoE4 antibody described herein. In some aspects, provided herein are isolated nucleic acid molecules comprising nucleotide sequences that encode an anti-ApoE4 antibody described herein. In some aspects, provided herein are isolated nucleic acid molecules comprising nucleotide sequences that encode an anti-ApoE4 alternative scaffold described herein.

Also provided are vectors comprising the nucleic acid molecules provided herein. In some aspects, the vector is an expression vector in which a nucleic acid molecule provided herein is operably linked to an expression control element. In embodiments where the ABP is an antibody, the heavy chain variable region and light chain variable regions may be contained in the same vector or in different vectors.

Also provided are host cells comprising the nucleic acid molecules provided herein and the vectors provided herein.

Also provided are methods of making an ABP provided herein by using a host cell provided herein or a cell-free expression system comprising a nucleic acid molecule or a vector provided herein. In certain embodiments, provided herein are methods of producing an anti-ApoE4 ABP by culturing a host cell provided herein under conditions that an ABP is produced. In certain embodiments, the method further includes recovering the anti-ApoE4 ABP produced by the host cell. Also provided is an ABP produced by the methods disclosed herein.

Also provided is a pharmaceutical composition comprising an anti-ApoE4 ABP provided herein and a pharmaceutically acceptable carrier.

Also provided are methods of preventing, treating or reducing the risk of a disease, condition or disorder associated with ApoE4 expression in a subject, comprising administering to the subject a therapeutically effective amount of an ABP provided herein or a pharmaceutical composition provided herein.

Also provided are methods of modulating one or more activities of, or phenotypes associated with, an ApoE4 protein or a lipoprotein particle comprising an ApoE4 protein in a subject, comprising administering to the subject a therapeutically effective amount of an ABP provided herein or a pharmaceutical composition provided herein.

In some embodiments, the methods further comprise administering to the subject a therapeutically effective amount of a second agent. In some embodiments, the second agent is selected from an amyloid beta directed therapeutic, a tau protein directed therapeutic, and combinations thereof. In certain embodiments, the second agent is selected from an antibody that binds a CD33 protein, an antibody that binds a sortilin protein, an antibody that binds a TREM2 protein, an antibody that binds an amyloid beta protein, an antibody that binds tau protein, a BACE inhibitor, a gamma secretase inhibitor, an agent that disaggregates amyloid beta oligomers, an agent that disaggregates tau fibrils, and combinations thereof.

In some embodiments, the ABP provided herein is administered by intravenous, intramuscular, intraperitoneal, intracerobrospinal, intracranial, intraarterial cerebral infusion, intracerebroventricular, intraspinal, subcutaneous, intra-articular, intrasynovial, intrathecal, oral, topical, or inhalation routes.

The methods provided herein find use in preventing, treating or reducing the risk of any disease, condition or disorder associated with ApoE4 expression. In some embodiments, the disease, disorder or condition is selected from dementia (e.g., frontotemporal dementia, vascular dementia), cognitive disorder, Alzheimer's disease (e.g., late onset Alzheimer's disease, familial Alzheimer's disease, sporadic form of Alzheimer's disease), cerebral amyloid angiopathy, traumatic brain injury, stroke, epilepsy, multiple sclerosis, and age-related macular degeneration. Other diseases, conditions or disorders associated with ApoE4 expression in a subject may include, for example, a cardiovascular disease, coronary heart disease (e.g., early-onset coronary heart disease), hypercholesterolemia, peripheral vascular disease, hypertriglyceridemia, hyperlipoproteinemia Type III, lipoprotein glomerulopathy and sea-blue histiocyte disease.

It is understood that each feature, embodiment or aspect, or combination thereof, described herein is meant to be combinable with any other feature, embodiment or aspect, or combination thereof, described herein. For example, where features are described with language such as “one embodiment,” “some embodiments,” “certain embodiments,” “further embodiment,” “specific exemplary embodiments,” and/or “another embodiment,” each of these types of embodiments is a non-limiting example of a feature that is intended to be combined with any other feature, or combination of features, described herein without having to list every possible combination, regardless of whether such combinations are actually written and drawing no implication from the writing of some combinations but not others. Such features or combinations of features apply to any of the aspects of the invention.

Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1 shows an amino acid sequence of a mature human ApoE4 protein.

FIG. 2 depicts the structure of certain LDL receptor family member proteins.

### DETAILED DESCRIPTION

### 1. General Techniques

Techniques and procedures described or referenced herein (e.g., for cloning and expressing nucleotide and polypeptide sequences, including for example antibody sequences) are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (RI. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993), each of which is incorporated herein by reference.

### 2. Definitions

As used herein, the terms “lipidated ApoE4” or “lipidated ApoE4 protein” refer to an ApoE4 protein that is bound to a lipid. The interaction between ApoE4 and the lipid is non-covalent. The lipid may be any suitable lipid that is bound by an ApoE4 protein. Suitable lipids include, for example, one or more of a triglyceride, a phospholipid, a sphingolipid, a cholesterol ester, cholesterol, DMPC, triolein, phosphatidylcholine, phosphatidylethanolamine, phosphatidylglycerol, phosphatidylserine, phosphatidylinositol, PIP, phosphatidic acid, and cardiolipin.

As used herein, the term “ApoE4 carrier” refers to a subject having at least one ε4 allele. In some aspects, the subject has one ε4 allele. In some aspects, the subject has two ε4 alleles. In some aspects, the subject is an ε4/4 homozygote. In some aspects, the subject is an ε4/3 heterozygote. In some aspects, the subject is an ε4/2 heterozygote.

As used herein the terms “preventing,” “prevention” and “prevent” include providing prophylaxis, either temporarily or permanently, either partially or completely, with respect to occurrence or recurrence of a particular disease, disorder, or condition in an subject. A subject may be predisposed to, susceptible to a particular disease, disorder, or condition, or at risk of developing such a disease, disorder, or condition, but not yet diagnosed with the disease, disorder, or condition. Such preventing need not be absolute to be useful.

As used herein, a subject “at risk” of developing a particular disease, disorder or condition may or may not have detectable disease or symptoms of disease (e.g., clinical symptoms), and may or may not have displayed detectable disease or symptoms of disease prior to the treatment methods described herein. “At risk” denotes that a subject has one or more risk factors (e.g., the presence of ApoE4), which are measurable parameters that correlate with development of a particular disease, disorder, or condition, as known in the art. A subject having one or more of these risk factors has a higher probability of developing a particular disease, disorder, or condition than a subject without one or more of these risk factors.

As used herein, the terms “treatment,” “treating” and “treat” refer to clinical intervention designed to alter the natural course of the individual being treated during the course of clinical pathology. Desirable effects of treatment include decreasing the rate of progression, eliminating, ameliorating or palliating the pathological state, and remission or improved prognosis of a particular disease, disorder, or condition, either temporarily or permanently, either partially or completely. An individual is successfully “treated,” for example, if one or more symptoms associated with a particular disease, disorder, or condition are mitigated or eliminated.

An “effective amount” refers to an amount effective, at dosages and for periods of time necessary, to achieve the desired therapeutic or prophylactic result. An effective amount can be provided in one or more administrations.

A “therapeutically effective amount” is an amount required to effect a measurable improvement in a symptom or the progression of a particular disease, disorder, or condition. A therapeutically effective amount herein may vary according to factors such as the disease state, age, sex, and weight of the patient, and the ability of the anti-ApoE4 ABP to elicit a desired response or effect in the subject. A therapeutically effective amount is also one in which any toxic or detrimental effects of the anti-ApoE4 ABP are outweighed by the therapeutically beneficial effects.

As used herein, administration “in conjunction” with another compound or composition (e.g., second agent) includes simultaneous administration and/or administration at different times. Administration in conjunction also encompasses administration as a co-formulation or administration as separate compositions, including at different dosing frequencies or intervals, and using the same route of administration or different routes of administration.

An “individual” or “subject” for purposes of treatment, prevention, or reducing risk (e.g., reduction of risk) refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Preferably, the individual or subject is human.

As used herein, the term “antigen-binding protein” (ABP) refers to a protein comprising one or more antigen-binding domains that specifically bind to an antigen. In some embodiments, the antigen-binding domain binds the antigen with specificity and affinity similar to that of an antibody. In some embodiments, the ABP comprises an antibody. In some embodiments, the ABP consists of an antibody. In some embodiments, the ABP consists essentially of an antibody. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment. An “ApoE4 ABP,” “anti-ApoE4 ABP,” or “ApoE4-specific ABP” is an ABP, as provided herein, which specifically binds to the antigen ApoE4. In certain embodiments, an ApoE4 ABP provided herein binds to an epitope of ApoE4 that is conserved between or among ApoE4 proteins from different species.

As used herein, the term “antigen-binding domain” means the portion of an ABP that is capable of specifically binding to an antigen. One example of an antigen-binding domain is an antigen-binding domain formed by a V_{H}-V_{L} dimer of an antibody. Another example of an antigen-binding domain is an antigen-binding domain formed by diversification of certain loops from the tenth fibronectin type III domain of an Adnectin.

The term “antibody” is used in the broadest sense and includes fully assembled antibodies, immunoglobulins, tetrameric antibodies, native antibodies, monoclonal antibodies, polyclonal antibodies, multispecific antibodies (e.g., bispecific antibodies), antibody fragments, and recombinant peptides comprising the forgoing. An antibody is one type of ABP.

As used herein, the term “alternative scaffold” refers to a non-antibody molecule in which one or more regions are diversified to produce one or more antigen-binding domains. In some embodiments, the antigen-binding domain binds the antigen or epitope with specificity and affinity similar to that of an antibody. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD₃ (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J. Biol. Chem., 2014, 289:14392-14398; each of which is incorporated by reference in its entirety. An alternative scaffold is one type of ABP.

An “immunoglobulin” or “native antibody” is usually a tetrameric glycoprotein of about 150,000 Daltons. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen binding. The carboxy-terminal portion of each chain defines a constant region primarily responsible for effector function. Light chains are classified as kappa (κ) or lambda (λ), based on the amino acid sequences of their constant domains. Heavy chains are classified as mu (μ), delta (δ), gamma (γ), alpha (α), or epsilon (ε), and define the antibody's isotype as IgM, IgD, IgG, IgA, or IgE, respectively. Within light and heavy chains, the variable and constant regions are joined by a “J” region of about 12 or more amino acids, with the heavy chain also including a “D” region of about 10 more amino acids (see generally, Fundamental Immunology, Ch. 7 (Paul, W., ed., 2nd ed. Raven Press, N.Y. (1989); Basic and Clinical Immunology, 8th Ed., Stites, D., Terr A., and Parslow, T. (eds.), Appleton & Lange, Norwalk, Conn., 1994, page 71 and Chapter 6, incorporated by reference in their entirety for all purposes). The variable regions of each light/heavy chain pair form the antibody binding site such that an intact immunoglobulin has two binding sites. Each light chain is linked to a heavy chain by one covalent disulfide bond, while the number of disulfide linkages varies among the heavy chains of different immunoglobulin isotypes.

Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain Particular amino acid residues are believed to form an interface between the light and heavy chain variable domains (Chothia et al., J. Mol. Biol. 196:901-917, 1987).

Immunoglobulin variable domains exhibit the same general structure of relatively conserved framework regions (FR) joined by three hypervariable regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk, (J. Mol. Biol. 196:901-917, 1987); Chothia et al., (Nature 342:878-883, 1989).

The hypervariable region of an antibody refers to the CDR amino acid residues of an antibody which are responsible for antigen-binding. The hypervariable region comprises amino acid residues from a CDR (e.g., residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, 5th Ed. Public Health Service, National Institutes of Health, Bethesda, Md. (1991)) and/or those residues from a hypervariable loop (e.g., residues 26-32 (L1), 50-52 (L2) and 91-96 (L3) in the light chain variable domain and 26-32 (H1), 53-55 (H2) and 96-101 (1-13) in the heavy chain variable domain as described by (Chothia et al., J. Mol. Biol. 196: 901-917 (1987)). CDRs have also been identified and numbered according to ImMunoGenTics (IMGT) numbering (Lefranc, M.-P., The Immunologist, 7, 132-136 (1999); Lefranc, M.-P. et al., Dev. Comp. Immunol., 27, 55-77 (2003), which describes the CDR locations in the light and heavy chain variable domains as follows: CDR1, approximately residues 27 to 38; CDR2, approximately residues 56 to 65; and, CDR3, approximately residues 105 to 116 (germline) or residues 105 to 117 (rearranged). In one embodiment, it is contemplated that the CDRs are located at approximately residues 26-31 (L1), 49-51 (L2) and 88-98 (L3) in the light chain variable domain and approximately residues 26-33 (H1), 50-58 (H2) and 97-111 (H3) in the heavy chain variable domain of an antibody heavy or light chain of approximately similar length to those disclosed herein. However, one of skill in the art understands that the actual location of the CDR residues may vary from the projected residues described above when the sequence of the particular antibody is identified. Framework or FR residues are those variable domain residues other than the hypervariable region residues.

An “isolated” ABP, such as an isolated ABP of the present disclosure that binds to an ApoE4 protein, is one that has been identified, separated and/or recovered from a component of its production environment (e.g., naturally or recombinantly). Preferably, the isolated ABP is free of association with all other contaminant components from its production environment. Contaminant components from its production environment, such as those resulting from recombinant transfected cells, are materials that would typically interfere with research, diagnostic, prophylactic or therapeutic uses for the ABP, and may include enzymes, hormones, and other proteinaceous or non-proteinaceous solutes. In preferred embodiments, the ABP will be purified: (1) to greater than 95% by weight of ABP as determined by, for example, the Lowry method, and in some embodiments, to greater than 96%, 97%, 98% or 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. An isolated ABP includes the ABP in situ within recombinant cells since at least one component of the ABP's natural environment will not be present. Ordinarily, however, an isolated ABP will be prepared by at least one purification step.

The “variable region” or “variable domain” of an antibody, such as an anti-ApoE4 antibody of the present disclosure, refers to the amino-terminal portion or domains of the heavy or light chain of the antibody. The variable domains of the heavy chain and light chain may be referred to as “V_{H}” and “V_{L},” respectively. These domains are generally the most variable parts of the antibody (relative to other antibodies of the same class) and are primarily responsible for antigen binding (e.g., contain the antigen binding sites).

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) or complementarity determining regions (CDRs), in both the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-ApoE4 antibody of the present disclosure, obtained from a population of substantially homogeneous antibodies, i.e., the individual antibodies comprising the population are identical except for possible naturally occurring mutations and/or post-translational modifications (e.g., isomerizations and amidations) that may be present in minor amounts. Monoclonal antibodies are each directed against the same epitope or epitopes. In contrast to polyclonal antibody preparations which typically include different antibodies directed against different epitopes, each monoclonal antibody is directed against a single determinant on the antigen. In addition to their specificity, monoclonal antibodies are advantageous in that they are, for example, synthesized by hybridoma culture or recombinantly produced (e.g., by transformed or transfected mammalian host cells), uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies of the present disclosure may be made by a variety of techniques, including, for example, the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” or “whole antibody” are used interchangeably to refer to an antibody, such as an anti-ApoE4 antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises an antigen-binding portion of an intact antibody, such as the variable region of the intact antibody. Examples of antibody fragments include Fab, Fab′, F(ab′)₂ and Fv fragments, diabodies, triabodies, tetrabodies, minibodies, linear antibodies (see U.S. Pat. No. 5,641,870, Example 2; Zapata et al., Protein Eng. 8(10):1057-1062 (1995)), single-chain antibodies (scFvs), certain multispecific antibodies formed from antibody fragments, domain antibodies (dAbs), nobodies, small modular immunopharmaceuticals (SMIPs), antigen-binding-domain immunoglobulin fusion proteins, camelized antibodies, VHH-containing antibodies, variants or derivatives of any of the foregoing, and polypeptides that contain at least a portion of an immunoglobulin that is sufficient to confer specific antigen binding to the polypeptide, such as one, two, three, four, five or six CDR sequences. Antibody fragments may be produced by recombinant DNA techniques or by enzymatic or chemical cleavage of intact antibodies.

Papain digestion of intact antibodies, such as intact anti-ApoE4 antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (VII), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Fv” is the minimum antibody fragment which contains a complete antigen binding site. This fragment consists of a dimer of one heavy chain variable region domain and one light chain variable region domain in tight, non-covalent association. From the folding of these two domains emanate six hypervariable loops (3 loops each from the heavy and light chains) that contribute the amino acid residues for antigen binding and confer antigen binding specificity to the antibody. However, even a single variable domain (or half of an Fv comprising only three HVRs specific for an antigen) may have the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains, which enables the sFv to form the desired structure for antigen binding. For a review of the sFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Nat'l Acad. Sci. USA 90:6444-48 (1993).

As used herein, a “chimeric antibody” refers to an antibody, such as a chimeric anti-ApoE4 antibody of the present disclosure, in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used to refer to a subset of “chimeric antibodies.”

“Humanized” forms of non-human (e.g., murine) antibodies, such as humanized forms of anti-ApoE4 antibodies of the present disclosure, are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody, such as an anti-ApoE4 antibody of the present disclosure, produced by a human or has made using any of the techniques for making human antibodies, such as those disclosed herein or known in the art. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Methods for display of peptides on the surface of yeast, microbial and mammalian cells can also be used to identify human antibodies (See, for example, U.S. Pat. Nos. 5,348,867; 5,723,287; 6,699,658; Wittrup, Curr Op. Biotech. 12:395-99 (2001); Lee et al, Trends in Biotech. 21(1) 45-52 (2003); Surgeeva et al, Adv. Drug Deliv. Rev. 58: 1622-54 (2006)). Additionally, human antibodies may be isolated using in vitro display methods and microbial cell display, including ribosome display and mRNA display (Amstutz et al, Curr. Op. Biotech. 12: 400-05 (2001)). Selection using ribosome display is described in Hanes et al., (Proc. Natl. Acad Sci USA, 94:4937-4942 (1997)) and U.S. Pat. Nos. 5,643,768 and 5,658,754.

Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain, such as that of an anti-ApoE4 antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the V_{L}, and 26-35 (H1), 50-65 or 49-65 (a preferred embodiment) (H2), and 93-102, 94-102, or 95-102 (H3) in the V_{H}. The variable-domain residues are numbered according to Kabat et al., supra, for each of these extended-HVR definitions.

“Framework” or “FR” residues are those variable domain residues other than the HVR residues as herein defined.

The phrase “variable-domain residue-numbering as in Kabat” or “amino-acid-position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. References to residue numbers in the variable domain of antibodies means residue numbering by the Kabat numbering system. References to residue numbers in the constant domain of antibodies means residue numbering by the EU numbering system (e.g., see United States Patent Publication No. 2010-280227).

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a V_{H}, preferable those changes occur at only three, two, or one of positions 71H, 73H and 78H; for instance, the amino acid residues at those positions may by 71A, 73T and/or 78A. In one embodiment, the V_{L} acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino-acid modification” at a specified position, e.g., of an anti-ApoE4 ABP of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” ABP, such as an affinity matured anti-ApoE4 ABP of the present disclosure, is one with one or more alterations in one or more antigen-binding domains (e.g., HVRs) thereof that result in an improvement in the affinity of the ABP for antigen, compared to a parent ABP that does not possess those alteration(s). In one embodiment, an affinity-matured ABP has nanomolar or even picomolar affinities for the target antigen. Affinity-matured antibodies are produced by various procedures known in the art. For example, Marks et al., Bio/Technology 10:779-783 (1992) describes affinity maturation of antibodies by V_{H}- and V_{L}-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, “specifically recognizes,” “specifically binds” or “binds specifically to” refers to measurable and reproducible interactions such as attraction or binding between a target and an ABP, such as an anti-ApoE4 ABP provided herein, that is determinative of the presence of the target in the presence of a heterogeneous population of molecules including biological molecules. For example, an ABP, such as an anti-ApoE4 ABP of the present disclosure, that specifically or preferentially binds to a target or an epitope is an ABP that binds this target or epitope with greater affinity, avidity, more readily, and/or with greater duration than it binds to other targets or other epitopes of the target. It is also understood by reading this definition that, for example, an ABP (or a moiety) that specifically or preferentially binds to a first target may or may not specifically or preferentially bind to a second target. As such, “specific binding” or “preferential binding” does not necessarily require (although it can include) exclusive binding. An ABP that specifically binds to a target may have an association constant of at least about 10³M⁻¹ or 10⁴M⁻¹, sometimes about 10⁵M⁻¹ or 10⁶ M⁻¹, in other instances about 10⁶ M⁻¹ or 10⁷M⁻¹, about 10⁸ M⁻¹ to 10⁹M⁻¹, or about 10¹⁰ M⁻¹ to 10¹¹ M⁻¹ or higher. Alternatively, an ABP that specifically binds to a target may exhibit binding affinity to the target antigen of a K_{d} of less than or equal to about 10⁻⁵ M, less than or equal to about 10⁻⁶ M, less than or equal to about 10⁻⁷ M, less than or equal to about 10⁻⁸ M, less than or equal to about 10⁻⁹ M, less than or equal to about 10⁻¹⁰ M, less than or equal to about 10⁻¹¹ M, or less than or equal to about 10⁻¹² M, or less. Such affinities may be readily determined using conventional techniques, such as by equilibrium dialysis; by using surface plasmon resonance (SPR) technology (e.g., the BIAcore 2000 instrument, using general procedures outlined by the manufacturer); by radioimmunoassay using ¹²⁵I-labeled target antigen; by KinExA kinetic exclusion assay (e.g., using general procedures for the KinExA device outlined by the manufacturer, Sapidyne Instruments, Inc., Boise, Id.; U.S. Pat. No. 6,664,114); or by another method set forth in the examples below or known to the skilled artisan. The affinity data may be analyzed, for example, by the method of Scatchard et al., (Ann N.Y. Acad. Sci., 51:660, 1949).

A variety of immunoassay formats can be used to select ABPs specifically immunoreactive with a particular protein. For example, solid-phase ELISA immunoassays are routinely used to select ABPs specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity

As used herein, an “interaction” between an ApoE4 protein, and a second molecule, such as for example a protein (e.g., glycoprotein) or a glycolipid (e.g., ganglioside) encompasses, for example, protein-protein interaction, a physical interaction, a chemical interaction, binding, covalent binding, and ionic binding. As used herein, an ABP “inhibits interaction” between two molecules (e.g., protein, glycolipid) when the ABP disrupts, reduces, or completely eliminates an interaction between the two molecules. An ABP of the present disclosure, or fragment thereof, “inhibits interaction” between two molecules when the ABP or fragment thereof binds to one of the two molecules (i.e., binds to an ApoE4 protein).

As used herein, the terms “modulates” and “modulating” refer to a change in the quality or quantity of a gene, protein, or any molecule that is inside, outside, or on the surface of a cell. In some aspects, the change can be an increase or decrease in expression or level of the molecule. In some aspects, the terms “modulates” and “modulating” also include changing the quality or quantity, positively or negatively, of a function/activity including, for example, intracellular signaling, cell-to-cell signaling, cell proliferation, cell survival, growth, adhesion, apoptosis, binding, chemotaxis, phagocytosis, internalization, clearance, recruitment, differentiation, and the like. In some aspects, the terms “modulates” and “modulating” refer to changing the function of, or phenotype associated with, an ApoE4 protein.

As used herein, the term “modulator” refers to a composition that modulates one or more physiological or biochemical events, such as an event associated with the activity of a molecule (e.g., target protein, ApoE4 protein), or with a disease, condition or disorder of the present disclosure. One example of a modulator of ApoE4 protein is an anti-ApoE4 ABP provided herein. In some embodiments, the modulator inhibits one or more biological activities associated a disease condition or disorder of the present disclosure. In some embodiments, the modulator increases one or more biological activities thereby ameliorating a symptom associated with a disease, condition or disorder of the present disclosure. In some embodiments, the modulator is an ABP, a peptide, a protein, an antibody or antibody fragment. In some embodiments, the modulator acts by blocking ligand binding or by competing for a ligand-binding site. In some embodiments, the modulator acts independently of ligand binding. In some embodiments the modulator does not compete for a ligand binding site. In some embodiments, the modulator blocks expression of a gene product involved in a disease, condition or disorder of the present disclosure. In some embodiments, the modulator blocks a physical interaction of two or more biomolecules involved in a disease, condition or disorder of the present disclosure. In some embodiments, modulators of the present disclosure inhibit one or more ApoE4 biological activities. In some embodiments, modulators of the present disclosure enhance or increase one or more ApoE4 biological activities. Modulators of the present disclosure may also inhibit interactions between ApoE4 and a ligand, such as a glycoprotein or glycolipid ligand. In some embodiments, modulators of the present disclosure may increase interactions between ApoE4 and a ligand.

An “agonist” ABP or an “activating” ABP is an ABP, such as an agonist anti-ApoE4 ABP of the present disclosure, that induces (e.g., increases) one or more activities or functions of a target antigen after the ABP binds the antigen. An “antagonist” ABP is used in the broadest sense, and includes an ABP, such as an antagonist anti-ApoE4 ABP of the present disclosure, that partially or fully blocks, inhibits, or neutralizes a biological activity of a target antigen after the ABP binds the antigen. Methods for identifying ABP agonists or antagonists may comprise contacting a target antigen of interest (e.g., ApoE4) with a candidate agonist or antagonist ABP and measuring a detectable change in one or more biological activities normally associated with the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Any suitable Fc region may be used in the ABPs provided herein. Suitable native-sequence Fc regions for use in the ABPs provided herein include human IgG1, IgG2, IgG3 and IgG4, but not limited to these Fc regions.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of ABPs.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

As used herein, “percent (%) amino acid sequence identity” and “homology” with respect to a peptide, polypeptide or ABP sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

An “isolated” nucleic acid molecule (e.g., an isolated nucleic acid molecule comprising a nucleotide sequence encoding a polypeptide, ABP, heavy or light chain of an antibody, heavy or light chain variable region of an antibody, or any portion thereof) is a nucleic acid molecule that is identified and separated from at least one contaminant nucleic acid molecule with which it is ordinarily associated in the environment in which it was produced. Preferably, the isolated nucleic acid molecule is free of association with all components associated with the production environment. Such isolated nucleic acid molecules are in a form or setting other than the form or setting in which they are found in nature. Isolated nucleic acid molecules therefore are distinguished from nucleic acids encoding the polypeptides and ABPs herein that exist naturally in cells, if any.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a viral vector, wherein additional DNA segments may be ligated into the viral genome. One type of viral vector is a phage vector. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes (e.g., nucleic acid molecules that encode the heavy or light chain of an antibody, such as an anti-ApoE4 antibody) to which they are operatively linked (e.g., operatively linked to an expression control element). Such vectors may be referred to herein as “recombinant expression vectors,” or simply, “expression vectors.” Frequently, expression vectors of utility in recombinant DNA techniques are often in the form of plasmids.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, and phosphoamidates, carbamates) and with charged linkages (e.g., phosphorothioates and phosphorodithioates), those containing pendant moieties, such as, for example, proteins (e.g., nucleases, toxins, antibodies, signal peptides, and poly-L-lysine), those with intercalators (e.g., acridine and psoralen), those containing chelators (e.g., metals, radioactive metals, boron, and oxidative metals), those containing alkylators, those with modified linkages (e.g., alpha anomeric nucleic acids), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “host cell” includes an individual cell or cell culture that can be or has been a recipient of exogenous vector(s) or nucleic acid(s). Host cells include progeny of a single host cell, and the progeny may not necessarily be completely identical (in morphology or in genomic DNA complement) to the original parent cell due to natural, accidental, or deliberate mutation. A host cell includes cells transfected, transformed or transduced with a polynucleotide or vector of this disclosure.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the invention described herein include “comprising,” “consisting,” and “consisting essentially of” aspects and embodiments.

### 3. ApoE4 Protein

In some aspects, the present disclosure provides ABPs that bind to a lipidated ApoE4 protein or a lipoprotein particle comprising an ApoE4 protein, such as for example, a lipoprotein particle that is a chylomicron, a high density lipoprotein (HDL) particle, an intermediate density lipoprotein (IDL) particle, a low density lipoprotein (LDL) particle or a very low density lipoprotein (VLDL) particle. In certain embodiments, the ABP modulates one or more ApoE4 activities after binding to a lipidated ApoE4 protein or a lipoprotein particle comprising an ApoE4 protein.

ApoE4 is variously referred to as APOE4, ApoE4, apoe4, Apolipoprotein E4, AD2, LDLCQ5 LPG, Alzheimer Disease 2 (APOE*E4-Associated, Late Onset), Apo-E4, Apolipoprotein E4, apolipoprotein E epsilon 4, ApoE-ε4 and e4.

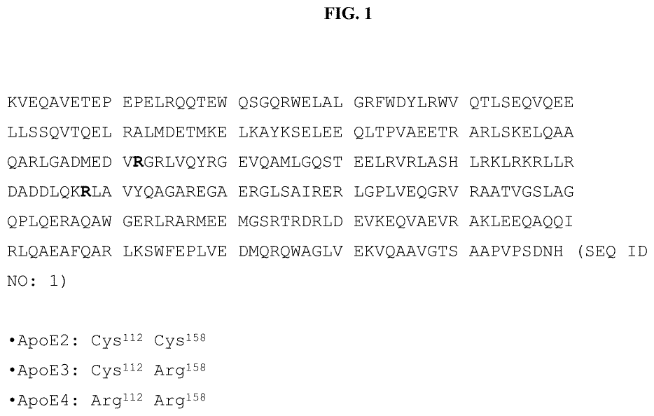

ApoE4 is 299 amino acids long (see FIG. 1; SEQ ID NO: 1) in its mature form and transports lipoproteins, fat-soluble vitamins, and cholesterol into the lymph system and then into the blood. It is synthesized principally in the liver, but has also been found in other tissues such as the brain, kidneys, and spleen. In the nervous system, non-neuronal cell types, most notably astroglia and microglia, are the primary producers of ApoE4, while neurons preferentially express the receptors for ApoE. There are seven currently identified mammalian receptors for ApoE4, which belong to the evolutionarily conserved low density lipoprotein receptor gene family.

ApoE4 is a member of a polymorphic family with three major isoforms: ApoE2 (cys112, cys158), ApoE3 (cys112, arg158), and ApoE4 (arg112, arg158). Although these allelic forms differ from each other by only one or two amino acids at positions 112 and/or 158, these differences alter ApoE structure and function. The ApoE proteins were initially recognized for their importance in lipoprotein metabolism and cardiovascular disease. Defects in ApoE are involved in familial dysbetalipoproteinemia, also known as type III hyperlipoproteinemia (HLP III), in which increased plasma cholesterol and triglycerides are the consequence of impaired clearance of chylomicron, VLDL and LDL remnants. More recently, ApoE proteins have been studied for their role in several biological processes not directly related to lipoprotein transport, including Alzheimer's disease (AD), immunoregulation, and cognition.

In the field of immune regulation, a number of studies point to ApoE's interaction with many immunological processes, including suppressing T cell proliferation, macrophage functioning regulation, lipid antigen presentation facilitation (by CD1) to natural killer T cells, as well as modulation of inflammation and oxidation.

ApoE4 is a known genetic risk factor for late-onset sporadic Alzheimer's disease (AD) in a variety of ethnic groups (Neurosciences (2012,) 17 (4): 321-6). Caucasian and Japanese carriers of two E4 alleles have between 10 and 30 times the risk of developing AD by 75 years of age, as compared to those not carrying any E4 alleles. While the mechanism of ApoE4's role remains to be fully determined, evidence suggests an interaction with amyloid (Neurosci. Lett. (1992) 135 (2): 235-238). Alzheimer's disease is characterized by build-ups of aggregates of the peptide beta-amyloid. Apolipoprotein E enhances proteolytic breakdown of this peptide, both within and between cells. ApoE4 appears to not be as effective as the other isoforms at catalyzing these reactions, potentially resulting in increased vulnerability to AD in individuals with that gene variation (Neuron (2008) 58 (5): 681-93). Linkage studies were followed by association analysis and demonstrated the ApoE4 allele as a strong genetic risk factor for AD (Am J Hum Genet (1991) 48:1034-50; Science (1993) 261: 921-3; Proc Natl Acad Sci (1993) 90: 1977-1981). Although 40-65% of AD patients have at least one copy of the APOE4 allele, ApoE4 is not a determinant of the disease, as at least a third of patients with AD are ApoE4 negative and some ApoE4 homozygotes never develop the disease. Yet those with two E4 alleles have up to 30 times the risk of developing AD. There are also data indicating that the ApoE2 allele may serve a protective role in AD (Nat. Genet. (1993)7; (2): 180-4). Thus, the genotype most at risk for developing Alzheimer's disease, and an earlier age of onset is ApoE 4,4. The ApoE 3,4 genotype is at increased risk, though not to the same degree as those homozygous for ApoE 4. The genotype ApoE 3,3 is considered at normal risk for AD. The genotype ApoE 2,3 is considered at lower risk for AD. Interestingly, people with copies of both the ApoE2 allele and the ApoE4 allele are at normal risk, similar to the ApoE 3,3 genotype.

As used herein, the term “disease-associated proteins or peptides” refers to a protein or peptide that is capable of forming an aggregate. In certain embodiments, the protein or peptide that is capable of forming an aggregate is selected from amyloid beta, tau, IAPP, TDP-43, alpha-synuclein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain, S-IBM, and combinations thereof. In certain embodiments, the disease-associated protein or peptide is selected from amyloid beta, alpha synuclein, tau, TDP-43, PrPSc, huntingtin, and combinations thereof.

### 4. Anti-ApoE4 Antigen-Binding Proteins

In some embodiments, the ApoE4 bound by the ABPs provided herein is human ApoE4 hApoE4 (SEQ ID NO: 1). In some embodiments, the ABPs provided herein also bind ApoE4 from one or more additional species. In some aspects, the one or more additional species are selected from Gorilla gorilla (Q9GLM8), Macaca mulatta (I2CYL7), Mus musculus (Q6GTX3), Rattus norvegicus (Q6PAH0), and Danio rerio (NM_131098.1).

In some embodiments, the ABPs provided herein comprise an immunoglobulin molecule. In some embodiments, the ABPs provided herein consist of an immunoglobulin molecule. In some embodiments, the ABPs provided herein consist essentially of an immunoglobulin molecule. In some aspects, the immunoglobulin molecule comprises an antibody. In some aspects, the immunoglobulin molecule consists of an antibody. In some aspects, the immunoglobulin molecule consists essentially of an antibody.

In some embodiments, the ABPs provided herein comprise a light chain. In some aspects, the light chain is a kappa light chain. In some aspects, the light chain is a lambda light chain.

In some embodiments, the ABPs provided herein comprise a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the ABPs provided herein comprise an antibody fragment. In some embodiments, the ABPs provided herein consist of an antibody fragment. In some embodiments, the ABPs provided herein consist essentially of an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment. In some aspects, the antibody fragment is a fragment of a single domain antibody. In some aspects, the antibody fragment is a diabody. In some aspects, the antibody fragment is a triabody. In some aspects, the antibody fragment is a tetrabody. In some aspects, the antibody fragment is a minibody. In some aspects, the antibody fragment is a linear antibody. In some aspects, the antibody fragment is a domain antibody. In some aspects, the antibody fragment is a nanobody. In some aspects, the antibody fragment is a SMIP. In some aspects, the antibody fragment is an antigen-binding-domain immunoglobulin fusion protein. In some aspects, the antibody fragment is a camelized antibody. In some aspects, the antibody fragment is a VHH-containing antibody.

In some embodiments, the ABPs provided herein are monoclonal antibodies. In some embodiments, the ABPs provided herein are polyclonal antibodies.

In some embodiments, the ABPs provided herein comprise a chimeric antibody. In some embodiments, the ABPs provided herein consist of a chimeric antibody. In some embodiments, the ABPs provided herein consist essentially of a chimeric antibody. In some embodiments, the ABPs provided herein comprise a humanized antibody. In some embodiments, the ABPs provided herein consist of a humanized antibody. In some embodiments, the ABPs provided herein consist essentially of a humanized antibody. In some embodiments, the ABPs provided herein comprise a human antibody. In some embodiments, the ABPs provided herein consist of a human antibody. In some embodiments, the ABPs provided herein consist essentially of a human antibody.

In some embodiments, the ABPs provided herein comprise an alternative scaffold. In some embodiments, the ABPs provided herein consist of an alternative scaffold. In some embodiments, the ABPs provided herein consist essentially of an alternative scaffold. Any suitable alternative scaffold may be used. In some aspects, the alternative scaffold is selected from an Adnectin™, an iMab, an Anticalin®, an EETI-II/AGRP, a Kunitz domain, a thioredoxin peptide aptamer, an Affibody®, a DARPin, an Affilin, a Tetranectin, a Fynomer, and an Avimer.

### 5. Affinity of Antigen-Binding Proteins for ApoE4

In some embodiments, the affinity of an ABP provided herein for ApoE4 as indicated by K_{d}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the ABP is between about 10⁻⁷ M and 10⁻¹² M. In some embodiments, the affinity of the ABP is between about 10⁻⁷ M and 10⁻¹¹ M. In some embodiments, the affinity of the ABP is between about 10⁻⁷ M and 10⁻¹⁰ M. In some embodiments, the affinity of the ABP is between about 10⁻⁷ M and 10⁻⁹ M. In some embodiments, the affinity of the ABP is between about 10⁻⁷ M and 10⁻⁸ M. In some embodiments, the affinity of the ABP is between about 10⁻⁸ M and 10⁻¹² M. In some embodiments, the affinity of the ABP is between about 10⁻⁸ M and 10⁻¹¹ M. In some embodiments, the affinity of the ABP is between about 10⁻⁹ M and 10⁻¹¹ M. In some embodiments, the affinity of the ABP is between about 10⁻¹⁰ M and 10⁻¹¹ M.

In some embodiments an ABP provided herein has a kₐ of at least about 10⁴ M⁻¹×sec⁻¹ when binding to ApoE4. In some embodiments the ABP has a kₐ of at least about 10⁵ M⁻¹×sec⁻¹. In some embodiments the ABP has a kₐ of at least about 10⁶ M⁻¹×sec⁻¹. In some embodiments the ABP has a kₐ of between about 10⁴ M⁻¹×sec⁻¹ and about 10⁵ M⁻¹×sec⁻¹. In some embodiments the ABP has a kₐ of between about 10⁵ M⁻¹×sec⁻¹ and about 10⁶ M⁻¹×sec⁻¹.

In some embodiments an ABP provided herein has a k_{d} of about 10⁻⁵ sec⁻¹ or less when binding to ApoE4. In some embodiments the ABP has a k_{d} of about 10⁻⁴ sec⁻¹ or less. In some embodiments the ABP has a k_{d} of about 10⁻³ sec⁻¹ or less. In some embodiments the ABP has a k_{d} of between about 10⁻² sec⁻¹ and about 10⁻⁵ sec⁻¹. In some embodiments the ABP has a k_{d} of between about 10⁻² sec⁻¹ and about 10⁻⁴ sec⁻¹. In some embodiments the ABP has a k_{d} of between about 10⁻³ sec⁻¹ and about 10⁻⁵ sec⁻¹.

In some embodiments, the anti-ApoE4 ABP binds a lipidated ApoE4 protein or a lipoprotein particle comprising an ApoE4 protein with a binding affinity greater than the binding affinity of the ABP for a non-lipidated ApoE4 protein. In some embodiments, the binding affinity of the ABP for a lipidated ApoE4 protein or a lipoprotein particle comprising an ApoE4 protein is at least 2-fold, at least 3-fold, at least 4-fold, at least 5-fold, at least 10-fold, at least 20-fold, at least 50-fold, at least 100-fold, at least 200-fold, at least 500-fold, at least 1000-fold, or at least 10,000-fold greater or more than the binding affinity of the ABP for a non-lipidated ApoE4 protein (as measured by lower K_{d}).

In some embodiments, the ABP binds specifically to a lipidated ApoE4 protein. In some embodiments, the ABP binds to a lipidated ApoE4 protein with greater affinity (e.g., preferentially, as measured by K_{d}) than the affinity of the ABP for an ApoE2 and/or ApoE3 protein. In some embodiments, the binding affinity of the ABP for a lipidated ApoE4 protein is at least 2-fold, at least 3-fold, at least 4-fold, at least 5-fold, at least 10-fold, at least 20-fold, at least 50-fold, at least 100-fold, at least 200-fold, at least 500-fold, at least 1000-fold, or at least 10,000-fold greater or more than the binding affinity of the ABP for an ApoE2 and/or ApoE3 protein (as measured by lower K_{d}).

### 6. Effects of Antigen-Binding Proteins on Functions of ApoE4 and Phenotypes Associated with ApoE4

In some embodiments, an anti-ApoE4 ABP provided herein modulates one or more functions of ApoE4 or a lipoprotein particle comprising ApoE4, or a phenotype associated with ApoE4 or a lipoprotein particle comprising ApoE4. In some aspects, the one or more functions of ApoE4, or phenotypes associated with ApoE4, are modulated to exhibit greater similarity to the corresponding function or phenotype associated with ApoE2 or a lipoprotein particle comprising an ApoE2 protein. In some aspects, the one or more functions of ApoE4, or phenotypes associated with ApoE4, are modulated to exhibit greater similarity to the corresponding function or phenotype associated with ApoE3 or a lipoprotein particle comprising an ApoE3 protein. Anti-ApoE4 ABPs of the present disclosure may be tested for one or more of the foregoing properties using procedures known in the art and/or described herein.

6.1. HDL and Phospholipid-Rich Particle Binding

In some embodiments, the anti-ApoE4 ABPs provided herein stabilize or increase the binding of a lipidated ApoE4 protein to an HDL particle or a phospholipid-rich lipid particle. In some embodiments, such increased binding is associated with decreased binding of ApoE4 to VLDL or triglyceride-rich particles. In some embodiments, the anti-ApoE4 ABP increases the distribution of a lipidated ApoE4 protein to HDL particles or phospholipid-rich lipid particles. In some embodiments, the anti-ApoE4 ABP increases the binding of a lipidated ApoE4 protein to an HDL particle or a phospholipid-rich lipid particle (e.g., in vitro or in a subject) by at least 10%, at least 20%, at least 30%, at least 40%, at least 50%, at least 70%, at least 90%, at least 100%, at least 125%, at least 150%, at least 175%, at least 200%, at least 300%, at least 400%, at least 500%, at least 1000% or more, for example, as compared to the binding of a lipidated ApoE4 protein to an HDL particle or a phospholipid-rich lipid particle (e.g., in vitro or in a subject) in the absence of the anti-ApoE4 ABP. In other embodiments, the anti-ApoE4 ABP increases the binding of a lipidated ApoE4 protein to an HDL particle or a phospholipid-rich lipid particle (e.g., in vitro or in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the binding of a lipidated ApoE4 protein to an HDL particle or a phospholipid-rich lipid particle (e.g., in vitro or in a subject) in the absence of the anti-ApoE4 ABP. In some embodiments, the binding of a lipidated ApoE4 protein to an HDL particle or a phospholipid-rich lipid particle in the presence of the ABP exhibits greater similarity to the binding of an ApoE2 protein to an HDL particle or a phospholipid-rich lipid particle. In some embodiments, the binding of a lipidated ApoE4 protein to an HDL particle or a phospholipid-rich lipid particle in the presence of the ABP exhibits greater similarity to the binding of an ApoE3 protein to an HDL particle or a phospholipid-rich lipid particle.

6.2. VLDL Particle and Triglyceride-Rich Lipid Particle Binding

In some embodiments, the anti-ApoE4 ABPs provided herein decreases the binding of a lipidated ApoE4 protein to a VLDL particle or a triglyceride-rich lipid particle. In some embodiments, such decreased binding is associated with increased binding to HDL or phospholipid-rich particles. In some embodiments, the anti-ApoE4 ABP decreases the distribution of a lipidated ApoE4 protein to VLDL particles or triglyceride-rich lipid particles. In some embodiments, the anti-ApoE4 ABP decreases the binding of a lipidated ApoE4 protein to a VLDL particle or a triglyceride-rich lipid particle (e.g., in vitro or in a subject) by at least 10%, at least 20%, at least 30%, at least 40%, at least 50%, at least 60%, at least 70%, at least 80%, at least 90%, at least 95%, at least 99% or more, for example, as compared to the binding of a lipidated ApoE4 protein to a VLDL particle or a triglyceride-rich lipid particle (e.g., in vitro or in a subject) in the absence of the anti-ApoE4 ABP. In other embodiments, the anti-ApoE4 ABP decreases the binding of a lipidated ApoE4 protein to a VLDL particle or a triglyceride-rich lipid particle (e.g., in vitro or in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the binding of a lipidated ApoE4 protein to a VLDL particle or a triglyceride-rich lipid particle (e.g., in vitro or in a subject) in the absence of the anti-ApoE4 ABP. In some embodiments, the binding of a lipidated ApoE4 protein to a VLDL particle or a triglyceride-rich lipid particle in the presence of the ABP exhibits greater similarity to the binding of an ApoE2 protein to a VLDL particle or a triglyceride-rich lipid particle. In some embodiments, the binding of a lipidated ApoE4 protein to a VLDL particle or a triglyceride-rich lipid particle in the presence of the ABP exhibits greater similarity to the binding of an ApoE3 protein to a VLDL particle or a triglyceride-rich lipid particle.

6.3. VLDL Particle Release

In some embodiments, the anti-ApoE4 ABPs provided herein increases the release of a lipidated ApoE4 protein from a VLDL particle. In some embodiments, the anti-ApoE4 ABP increases the release of a lipidated ApoE4 protein from a VLDL particle (e.g., in vitro or in a subject) by at least 10%, at least 20%, at least 30%, at least 40%, at least 50%, at least 70%, at least 90%, at least 100%, at least 125%, at least 150%, at least 175%, at least 200%, at least 300%, at least 400%, at least 500%, at least 1000% or more, for example, as compared to the release of a lipidated ApoE4 protein from a VLDL particle (e.g., in vitro or in a subject) in the absence of the anti-ApoE4 ABP. In other embodiments, the anti-ApoE4 ABP may increase the release of a lipidated ApoE4 protein from a VLDL particle (e.g., in vitro or in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the release of a lipidated ApoE4 protein from a VLDL particle in the absence of the anti-ApoE4 ABP. In some embodiments, the release of a lipidated ApoE4 protein from a VLDL particle in the presence of the ABP exhibits greater similarity to the release of an ApoE2 protein from a VLDL particle. In some embodiments, the release of a lipidated ApoE4 protein from a VLDL particle in the presence of the ABP exhibits greater similarity to the release of an ApoE3 protein from a VLDL particle.

6.4. ApoE4 Binding to LDLR and Other Members of the LDLR Protein Family

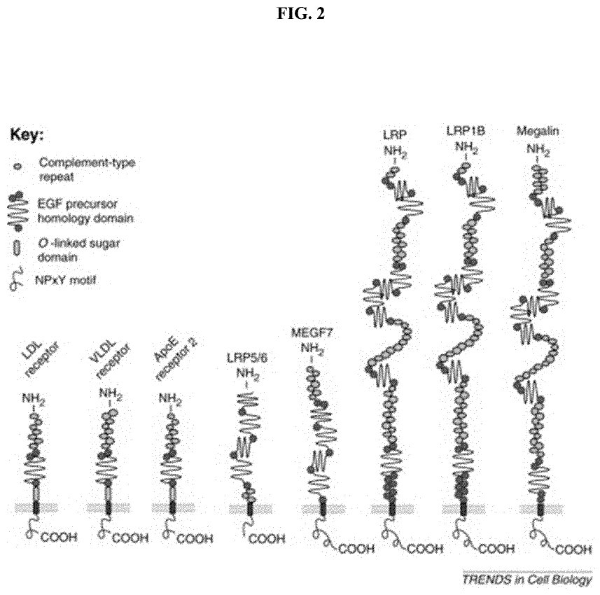

In some embodiments, the anti-ApoE4 ABPs provided herein decrease the binding of a lipidated ApoE4 protein to an LDLR or to one or more other members of the LDLR protein family. In some embodiments, the ABPs decrease the affinity of the ApoE4 protein for LDLR or the one or more other members of the LDLR protein family. In some aspects, the ABPs block the interaction between ApoE4 and LDLR or the one or more other members of the LDLR protein family. In some embodiments, the one or more other members of the LDLR protein family are selected from LDLR, VLDLR, LRP1, LRP1b, LRP2, LRP3, LRP4, LRP5, LRP6, LRP7, LRP8, LRP10, LRP11, LRP12 sortilin, and combinations thereof (see e.g., FIG. 2 for illustration of certain members).

In some embodiments, the ABP decreases the binding or weakens the affinity of a lipidated ApoE4 protein to an LDLR and/or to one or more other members of the LDLR protein family (e.g., in vitro or in a subject) by at least 10%, at least 20%, at least 30%, at least 40%, at least 50%, at least 60%, at least 70%, at least 80%, at least 90%, at least 95%, at least 99% or more, for example, as compared to the binding or affinity of a lipidated ApoE4 protein to an LDLR in the absence of the anti-ApoE4 ABP. In other embodiments, the anti-ApoE4 ABP decreases the binding or weakens the affinity of a lipidated ApoE4 protein to an LDLR (e.g., in vitro or in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the binding or affinity of a lipidated ApoE4 protein to an LDLR in the absence of the anti-ApoE4 ABP. In some embodiments, the binding or affinity of a lipidated ApoE4 protein to an LDLR or one or more other members of the LDLR protein family in the presence of the ABP exhibits greater similarity to the binding of an ApoE2 protein to such LDLR or other LDLR protein family member. In some embodiments, the binding or affinity of a lipidated ApoE4 protein to an LDLR or one or more other members of the LDLR protein family in the presence of the ABP exhibits greater similarity to the binding of an ApoE3 protein to such LDLR or other LDLR protein family member.

6.5. HSPG Binding

In some embodiments, the anti-ApoE4 ABPs provided herein enhance or strengthen (e.g., increase) the binding affinity of a lipidated ApoE4 protein to an HSPG. In some embodiments, the anti-ApoE4 ABP increases the binding affinity of a lipidated ApoE4 protein to an HSPG (e.g., in vitro or in a subject) by at least 10%, at least 20%, at least 30%, at least 40%, at least 50%, at least 70%, at least 90%, at least 100%, at least 125%, at least 150%, at least 175%, at least 200%, at least 300%, at least 400%, at least 500%, at least 1000% or more, for example, as compared to the binding affinity of a lipidated ApoE4 protein to an HSPG (e.g., in vitro or in a subject) in the absence of the anti-ApoE4 ABP. In other embodiments, the anti-ApoE4 ABP increases the binding affinity of a lipidated ApoE4 protein to an HSPG (e.g., in vitro or in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the binding affinity of a lipidated ApoE4 protein to an HSPG (e.g., in vitro or in a subject) in the absence of the anti-ApoE4 ABP. In some embodiments, the binding affinity of a lipidated ApoE4 protein for an HSPG exhibits greater similarity in the presence of the ABP to the binding affinity of an ApoE2 protein for an HSPG. In some embodiments, the binding affinity of a lipidated ApoE4 protein for an HSPG exhibits greater similarity in the presence of the ABP to the binding affinity of an ApoE3 protein for an HSPG.

6.6. APP Processing to Amyloid Beta

In some embodiments, the anti-ApoE4 ABPs provided herein reduce (e.g., prevent) processing of APP to amyloid beta. In some embodiments, the anti-ApoE4 ABPs reduce or decrease APP processing to amyloid beta (e.g., in vitro or in a subject) by at least 10%, at least 20%, at least 30%, at least 40%, at least 50%, at least 60%, at least 70%, at least 80%, at least 90%, at least 95%, at least 99% or more, for example, as compared to the APP processing (e.g., in vitro or in a subject) in the absence of the anti-ApoE4 ABP. In other embodiments, the anti-ApoE4 ABPs reduce or decrease APP processing to amyloid beta (e.g., in vitro or in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the processing of APP (e.g., in vitro or in a subject) in the absence of the anti-ApoE4 ABP. In some embodiments, the processing of APP to amyloid beta exhibits greater similarity in the presence of the ABP to the processing of APP to amyloid beta when the ApoE2 protein is present. In some embodiments, the processing of APP to amyloid beta exhibits greater similarity in the presence of the ABP to the processing of APP to amyloid beta when the ApoE3 protein is present.

6.7. Amyloid Beta Clearance

In some embodiments, the anti-ApoE4 ABPs provided herein may increase clearance (e.g., reduce accumulation) of amyloid beta in the brain. In some embodiments, the anti-ApoE4 ABP may increase clearance of amyloid beta in the brain (e.g., in a subject) by at least 10%, at least 20%, at least 30%, at least 40%, at least 50%, at least 70%, at least 90%, at least 100%, at least 125%, at least 150%, at least 175%, at least 200%, at least 300%, at least 400%, at least 500%, at least 1000% or more, for example, as compared to the clearance of amyloid beta in the brain in the absence of the anti-ApoE4 ABP. In other embodiments, an anti-ApoE4 ABP of the present disclosure may clearance of amyloid beta in the brain (e.g., in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the clearance of amyloid beta in the brain (e.g., in a corresponding subject) in the absence of the anti-ApoE4 ABP. In some embodiments, the clearance of Amyloid beta in the brain exhibits greater similarity in the presence of the ABP to the clearance of Amyloid beta in the brain when the ApoE2 protein is present. In some embodiments, the clearance of Amyloid beta in the brain exhibits greater similarity in the presence of the ABP to the clearance of Amyloid beta in the brain when the ApoE3 protein is present.

6.8. Blood-Brain Barrier Leakage

In some embodiments, the anti-ApoE4 ABPs provided herein reduce (e.g., prevent) blood-brain barrier leakage. In some embodiments, the anti-ApoE4 ABP reduces or decrease blood-brain barrier leakage (e.g., in a subject) by at least 10%, at least 20%, at least 30%, at least 40%, at least 50%, at least 60%, at least 70%, at least 80%, at least 90%, at least 95%, at least 99% or more, for example, as compared to blood-brain barrier leakage in the absence of the anti-ApoE4 ABP. In other embodiments, the anti-ApoE4 ABP reduces or decreases blood-brain barrier leakage (e.g., in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the blood-brain barrier leakage in the absence of the anti-ApoE4 ABP.

6.9. Formation of Neurofibrillary Tangles

In some embodiments, the anti-ApoE4 ABPs provided herein reduce (e.g., decrease, prevent) formation of neurofibrillary tangles. In some embodiments, the anti-ApoE4 ABP reduces formation of neurofibrillary tangles (e.g., in a subject) by at least 10%, at least 20%, at least 30%, at least 40%, at least 50%, at least 60%, at least 70%, at least 80%, at least 90%, at least 95%, at least 99% or more, for example, as compared to formation of neurofibrillary tangles in the absence of the anti-ApoE4 ABP. In other embodiments, the anti-ApoE4 ABP reduces formation of neurofibrillary tangles (e.g., in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the formation of neurofibrillary tangles in the absence of the anti-ApoE4 ABP.

6.10. Accelerated Aging

In some embodiments, an anti-ApoE4 ABP provided herein reduces (e.g., decreases, prevents) accelerated aging as measured by age-dependent length of telomeres. In some embodiments, the anti-ApoE4 ABP reduces accelerated aging as measured by age-dependent length of telomeres (e.g., in a subject) by at least 10%, at least 20%, at least 30%, at least 40%, at least 50%, at least 60%, at least 70%, at least 80%, at least 90%, at least 95%, at least 99% or more, for example, as compared to accelerated aging as measured by age-dependent length of telomeres in the absence of the anti-ApoE4 ABP. In other embodiments, the anti-ApoE4 ABP reduces accelerated aging as measured by age-dependent length of telomeres (e.g., in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared accelerated aging as measured by age-dependent length of telomeres in the absence of the anti-ApoE4 ABP.

Table 1 shows various functions of ApoE proteins (or lipoproteins comprising them), or phenotypes associated therewith, along with the effect of treatment with the ABPs provided herein on each function or effect. Except as otherwise indicated, the function or phenotype of ApoE2 is used as a baseline. Except as otherwise indicated, the change in function or phenotype in the ApoE4 column is relative to ApoE2 (or a lipoprotein comprising ApoE2). For example, “Increased” processing of APP to amyloid beta in the ApoE4 column indicates that such processing is increased relative to ApoE2 (or a lipoprotein comprising ApoE2).

The effects of treatment, in vitro or in a subject, with the ABPs provided herein are provided in the last column of Table 1.

An “increase” in a function or phenotype indicates that, in some embodiments, the ApoE4 function or phenotype associated with it is increased (in vitro or in a subject) by at least 10%, at least 20%, at least 30%, at least 40%, at least 50%, at least 70%, at least 90%, at least 100%, at least 125%, at least 150%, at least 175%, at least 200%, at least 300%, at least 400%, at least 500%, at least 1000% or more, for example, as compared to the function or phenotype in the absence of the anti-ApoE4 ABP. In other embodiments, such function or phenotype is increased (in vitro or in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the function or phenotype in the absence of the anti-ApoE4 ABP. In some embodiments, the function or phenotype exhibits greater similarity in the presence of the ABP to the function or phenotype of an ApoE2 protein. In some embodiments, the function or phenotype exhibits greater similarity in the presence of the ABP to the function or phenotype of an ApoE3 protein.

A “decrease” in a function or phenotype indicates that, in some embodiments, the ApoE4 function or phenotype associated with it is decreased (in vitro or in a subject) by at least 10%, at least 20%, at least 30%, at least 40%, at least 50%, at least 60%, at least 70%, at least 80%, at least 90%, at least 95%, at least 99% or more, for example, as compared to the function or phenotype in the absence of the anti-ApoE4 ABP. In other embodiments, such function or phenotype is decreased (in vitro or in a subject) by at least 1.5-fold, at least 2.0-fold, at least 3.0-fold, at least 4.0-fold, at least 5.0-fold, at least 7.5-fold, at least 10-fold, at least 20-fold, at least 50-fold, at least 100-fold, at least 200-fold, at least 500-fold, at least 1000-fold or more, for example, as compared to the function or phenotype in the absence of the anti-ApoE4 ABP. In some embodiments, the function or phenotype exhibits greater similarity in the presence of the ABP to the function or phenotype of an ApoE2 protein. In some embodiments, the function or phenotype exhibits greater similarity in the presence of the ABP to the function or phenotype of an ApoE3 protein.

In some embodiments, an ABP provided herein modulates one or more functions of ApoE4, or phenotypes associated with ApoE4, from among the functions and phenotypes provided in Table 1, as indicated in Table 1. In some embodiments, an ABP provided herein modulates two or more functions of ApoE4, or phenotypes associated with ApoE4, from among the functions and phenotypes provided in Table 1, as indicated in Table 1. In some embodiments, an ABP provided herein modulates three or more functions of ApoE4, or phenotypes associated with ApoE4, from among the functions and phenotypes provided in Table 1, as indicated in Table 1. In some embodiments, an ABP provided herein modulates more than three functions of ApoE4, or phenotypes associated with ApoE4, from among the functions and phenotypes provided in Table 1, as indicated in Table 1.

TABLE 1
Functions of, and phenotypes associated with, lipidated ApoE4 and comparison to lipidated ApoE2.

| Function of | | | |
|---|---|---|---|
| or | | | |
| Phenotype Associated with ApoE | | | Effect of Treatment with ApoE4 |
| Protein or Lipoprotein Particle | Lipidated | Lipidated | Antigen-Binding Protein |
| Comprising ApoE Protein | ApoE2 | ApoE4 | Provided Herein |
| HDL and Phospholipid-Rich Particle | Favored | Not Favored | Increases binding of lipidated |
| Binding | | | ApoE4 to HDL or a phospholipid |
| | | | rich lipid particle |
| VLDL and Triglyceride-Rich | Not Favored | Favored | Reduces binding of lipidated |
| Particle Binding | | | ApoE4 to VLDL or a triglyceride |
| | | | rich lipid particle, or increases the |
| | | | release of ApoE4 from such |
| | | | particles |
| Binding of LDLR or LDLR Family | Reduced to | Increased | Reduces binding of lipidated |
| Members | 2% of | relative to | ApoE4 to LDLR or LDLR family |
| | ApoE3 | ApoE3 and | members |
| | | ApoE2 | |
| Binding of Atypical LDLR Family | Normal | Normal | Binding to atypical LDLR family |
| Members | | | members is preserved |
| HSPG Binding | Greater than | Reduced | Increases binding of ApoE4 to |
| | ApoE4 | Compared to | HSPG |
| | | ApoE2 | |
| Processing of APP to Amyloid Beta | Normal | Increased | Reduces ApoE4-associated |
| | | | processing of APP to amyloid beta |
| Rate of Clearance of Amyloid Beta | Normal | Decreased | Reduces ApoE4-associated |
| | | | inhibition of amyloid beta |
| | | | clearance |
| Blood-Brain Barrier (BBB)Leakage | Normal | Increased | Reduces ApoE4-associated BBB |
| | | | leakage |
| Formation of Neurofibrillary | Normal | Increased | Reduces ApoE4-associated |
| Tangles | | | formation of neurofibrillary |
| | | | tangles |
| Inflammation | Normal | Increased | Reduces ApoE4-associated |
| | | | inflammation |
| Production of Amyloid Beta | Normal | Increased | Reduces ApoE4-associated |
| | | | production of amyloid beta |
| Clearance of Amyloid Beta from the | Normal | Decreased | Reduces ApoE4-associated |
| CNS by Transport Across the | | | reduction in clearance of amyloid |
| Blood-Brain Barrier | | | beta across the blood-brain barrier, |
| | | | or increasing clearance of amyloid |
| | | | beta across the BBB |
| Accumulation of Amyloid Beta in | Normal | Increased | Reducing ApoE4-associated |
| Tissue | | | accumulation of amyloid beta in |
| | | | tissue, or increasing clearance of |
| | | | amyloid beta from a tissue |
| Level of Intraneuronal Amyloid | Normal | Increased | Reduces ApoE4-associated |
| Beta | | | intraneuronal accumulation of |
| | | | amyloid beta |
| Internalization of Amyloid Beta into | Normal | Increased | Reduces ApoE4-associated |
| Nerve Cells | | | internalization of amyloid beta |
| | | | into nerve cells |
| Binding to Amyloid Beta and | No | Yes | Reduces the ApoE4-associated |
| Stabilization of Amyloid Beta, | | | stabilization of amyloid beta and |
| Resulting in the Accumulation of | | | the formation of amyloid beta |
| Multimers of Amyloid Beta | | | multimers |
| LDL Cholesterol Levels in Blood or | Normal | Increased | Reduces ApoE4-associated |
| Plasma | | | increase in LDL cholesterol |
| Clinically Undesirable Lipid Profile | No | Yes | Reduces ApoE4-associated |
| (e.g., hypercholesterolemia, high | | | clinically undesirable lipid profile |
| total cholesterol, high LDL) | | | |
| LDLR Levels on Cell Surfaces | Normal | Decreased | Reduces ApoE4-associated |
| | | | downregulation of LDLR on cell |
| | | | surfaces |
| LDLR Protein Family Member | Normal | Decreased | Reduces ApoE4-associated |
| Levels on Cell Surfaces | | | downregulation of LDLR protein |
| | | | family members on cell surfaces |
| Recovery from Traumatic or Non- | Normal | Delayed | Reduces ApoE4-associated |
| Traumatic Acquired Brain Injury | | | delayed recovery from traumatic |
| (e.g., Head Trauma, Cerebral | | | or non-traumatic acquired brain |
| Hemorrhage, Stroke or Epilepsy) | | | injury |
| Risk of Developing Alzheimer's | Decreased | Increased | Reduces ApoE4-associated risk of |
| Disease or Late Onset Alzheimer's | Compared | Compared to | developing Alzheimer's disease or |
| Disease, or Symptoms or Pathology | to ApoE3 | ApoE3 and | late onset Alzheimer's disease, or |
| Thereof | | ApoE2 | symptoms or pathology thereof |
| Risk of Developing Cardiovascular | Decreased | Increased | Reduces ApoE4-associated risk of |
| Disease, or Symptoms or Pathology | Compared | Compared to | developing cardiovascular disease |
| Thereof | to ApoE3 | ApoE3 and | or symptoms or pathology thereof |
| | | ApoE2 | |
| Risk of Developing Coronary Heart | Decreased | Increased | Reduces ApoE4-associated risk of |
| Disease, or Symptoms or Pathology | Compared | Compared to | developing coronary artery disease |
| Thereof | to ApoE3 | ApoE3 and | or symptoms or pathology thereof |
| | | ApoE2 | |
| Risk of Developing Atherosclerosis, | Decreased | Increased | Reduces ApoE4-associated risk of |
| or Symptoms or Pathology Thereof | Compared | Compared to | developing atherosclerosis or |
| | to ApoE3 | ApoE3 and | symptoms or pathology thereof |
| | | ApoE2 | |
| Risk of Developing Peripheral | Normal | Increased | Reduces ApoE4-associated risk of |
| Vascular Disease, or Symptoms or | | | developing peripheral vascular |
| Pathology Thereof | | | disease or symptoms or pathology |
| | | | thereof |
| Risk of Developing Dementia, or | Normal | Increased | Reduces ApoE4-associated risk of |
| Symptoms or Pathology Thereof | | | developing dementia or symptoms |
| | | | or pathology thereof |
| Risk of Developing Vascular | Normal | Increased | Reduces ApoE4-associated risk of |
| Dementia, or Symptoms or | | | developing vascular dementia or |
| Pathology Thereof | | | symptoms or pathology thereof |
| Risk of Developing Frontotemporal | Normal | Increased | Reduces ApoE4-associated risk of |
| Dementia, or Symptoms or | | | developing frontotemporal |
| Pathology Thereof | | | dementia or symptoms or |
| | | | pathology thereof |
| Risk of Developing Cerebral | Normal | Increased | Reduces ApoE4-associated risk of |
| Amyloid Angiopathy, or Symptoms | | | developing cerebral amyloid |
| or Pathology Thereof | | | angiopathy or symptoms or |
| | | | pathology thereof |
| Risk of Developing Multiple | Normal | Increased | Reduces ApoE4-associated risk of |
| Sclerosis, or Symptoms or | | | developing multiple sclerosis or |
| Pathology Thereof | | | symptoms or pathology thereof |
| Risk of Developing Age-Related | Normal | Increased | Reduces ApoE4-associated risk of |
| Macular Degeneration, or | | | developing age-related macular |
| Symptoms or Pathology Thereof | | | degeneration or symptoms or |
| | | | pathology thereof |
| Rate of Aging | Normal | Increased | Reduces ApoE4-associated |
| | | | acceleration of aging |
| Cognitive Impairment | No | Yes | Reduces ApoE4-associated |
| | | | cognitive impairment, or |
| | | | normalizes cognitive function in a |
| | | | subject expressing ApoE4 |
| Phagocytosis in Microglia, | Normal | Decreased | Reduces ApoE4-associated |
| Macrophages, Monocytes or | | | inhibition of phagocytosis in |
| Astrocytes | | | microglia, macrophages, |
| | | | monocytes, or astrocytes |
| Uptake of Soluble Amyloid Beta by | Normal | Decreased | Reduces ApoE4-associated |
| Astrocytes | | | decrease in soluble amyloid beta |
| | | | uptake by astrocytes |
| Myelin Cholesterol Levels | Normal | Depleted | Reduces ApoE4-associated |
| | | | depletion of myelin cholesterol |
| Adverse Drug Reaction to Statin | No | Yes | Reduces ApoE4-associated |
| Therapy or Poor Responsiveness to | | | adverse drug reaction to statin |
| Statin Therapy | | | therapy or poor responsiveness to |
| | | | statin therapy |
| Pathological Alzheimer's Disease- | No | Yes | Reduces ApoE4-associated |
| like Gene Expression Profile | | | aberrant gene expression profiles |
| | | | associated with Alzheimer's |
| | | | disease |
| Glucose Metabolism in Brains of | Normal | Decreased | Reduces ApoE4-associated |
| Pre-Symptomatic Alzheimer's | | | reduction in glucose metabolism |
| Disease Patients | | | in brains of pre-symptomatic |
| | | | Alzheimer's disease patients |
| Volume of Brain Structures in Pre- | Normal | Decreased | Reduces ApoE4-associated |
| Symptomatic Alzheimer's Patients | | | reduction in volume of brain |
| | | | structures in pre-symptomatic |
| | | | Alzheimer's disease patients |
| Senile Plaque Formation | Normal | Increased | Reduces ApoE4-associated senile |
| | | | plaque formation |
| Uptake of Amyloid Beta by | Normal | Decreased | Reduces ApoE4-associated |
| Neurons, Astroglia, Microglia, | | | decrease in amyloid beta uptake |
| Oligodendroglia, Endothelial Cells | | | by neurons, astroglia, microglia, |
| | | | oligodendroglia or endothelial |
| | | | cells |
| Pathological Microglial Activity | No | Yes | Reduces ApoE4-associated |
| (including one or more of increased | | | pathological microglial activity |
| inflammatory polarization or | | | |
| decreased repair functions and | | | |
| decreased phagocytosis) | | | |
| Competing with Soluble Amyloid | No | Yes | Reduces the binding of ApoE4 to |
| Beta for Low-Density Lipoprotein | | | LRP1, thereby decreasing |
| Receptor-Related Protein 1 (LRP1)- | | | ApoE4's ability to compete with |
| Dependent Cellular Uptake by | | | soluble amyloid beta for binding |
| Astrocytes | | | to LRP1 |
| Clearance of Apoptotic Neurons, | Normal | Decreased | Reduces ApoE4-associated |
| Nerve Tissue Debris, Non-Nerve | | | reduction in clearance of apoptotic |
| Tissue Debris, Bacteria, Foreign | | | neurons, nerve tissue debris, non- |
| Bodies, or Disease-Associated | | | nerve tissue debris, bacteria, |
| Proteins or Peptides | | | foreign bodies, or disease- |
| | | | associated proteins or peptides. |
| Phagocytosis of Apoptotic Neurons, | Normal | Decreased | Reduces ApoE4-associated |
| Nerve Tissue Debris, Non-Nerve | | | reduction in phagocytosis of |
| Tissue Debris, Bacteria, Foreign | | | apoptotic neurons, nerve tissue |
| Bodies, or Disease-Associated | | | debris, non-nerve tissue debris, |
| Proteins or Peptides | | | bacteria, foreign bodies, or |
| | | | disease-associated proteins or |
| | | | peptides. |

### 7. Preparation of Antigen-Binding Proteins

Anti-ApoE4 ABPs of the present disclosure include alternative scaffolds, polyclonal antibodies, monoclonal antibodies, humanized and chimeric antibodies, human antibodies, antibody fragments (e.g., antigen binding fragments, Fab, Fab′-SH, Fv, scFv, and F(ab′)2), bispecific and polyspecific antibodies, multivalent antibodies, library-derived antibodies, antibodies having modified effector functions, fusion proteins containing an antibody portion, and any other modified configuration of the immunoglobulin molecule that includes an antigen binding site, including glycosylation variants, amino acid sequence variants, and covalently modified variants of the foregoing. The anti-ApoE4 antibodies may be human, murine, rat, or of any other origin (including chimeric or humanized antibodies).

7.1. Polyclonal Antibodies

Polyclonal antibodies, such as anti-ApoE4 polyclonal antibodies, are generally raised in animals by multiple immunizations (e.g., subcutaneous (sc) injection, intraperitoneal (ip) injection) of the relevant antigen and an adjuvant. It may be useful to conjugate the relevant antigen (e.g., a purified or recombinant ApoE4 protein of the present disclosure) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R′ are independently lower alkyl groups. Examples of adjuvants which may be employed include, for example, Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 μg (for rabbits) or 5 μg (for mice) of the protein or conjugate with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant-cell culture as protein fusions. Also, aggregating agents such as alum are suitable to enhance the immune response.

7.2. Monoclonal Antibodies

Monoclonal antibodies, such as anti-ApoE4 monoclonal antibodies, are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being from a mixture of discrete antibodies.

For example, the anti-ApoE4 monoclonal antibodies may be made using the hybridoma method first described by Köhler et al., Nature, 256:495 (1975), or may be made by recombinant DNA methods (e.g., U.S. Pat. No. 4,816,567).

In the hybridoma method, a mouse or other appropriate host animal, such as a hamster, is immunized as hereinabove described to elicit lymphocytes that produce or are capable of producing antibodies that will bind (e.g., specifically bind) to the protein used for immunization (e.g., a purified or recombinant ApoE4 protein (e.g., lipidated ApoE4) of the present disclosure). Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The immunizing agent will typically include the antigenic protein (e.g., a purified or recombinant ApoE4 protein (e.g., lipidated ApoE4 protein) or lipoprotein particle comprising an ApoE4 protein of the present disclosure) or a fusion variant thereof. Generally peripheral blood lymphocytes (“PBLs”) are used if cells of human origin are desired, while spleen or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, such as myeloma cells of rodent, bovine or human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient-cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors (available from the Salk Institute Cell Distribution Center, San Diego, Calif. USA), as well as SP-2 cells and derivatives thereof (e.g., X63-Ag8-653) (available from the American Type Culture Collection, Manassas, Va. USA). Human myeloma and mouse-human heteromyeloma cell lines have also been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen (e.g., a lipidated ApoE4 protein of the present disclosure). Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen (e.g., a lipidated ApoE4 protein of the present disclosure). Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Anti-ApoE4 monoclonal antibodies may also be made by recombinant DNA methods, such as those disclosed in U.S. Pat. No. 4,816,567, and as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host-cells such as E. coli cells, simian COS cells, Chinese hamster ovary (CHO) cells, or myeloma cells that do not otherwise produce immunoglobulin protein, in order to synthesize monoclonal antibodies in such recombinant host-cells. Review articles on recombinant expression in bacteria of DNA encoding the antibody include Skerra et al., Curr. Opin. Immunol., 5:256-262 (1993) and Plückthun, Immunol. Rev. 130:151-188 (1992).

In certain embodiments, anti-ApoE4 antibodies can be isolated from antibody phage libraries, such as for example, generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies of desired specificity (e.g., those that bind an ApoE4 protein (e.g., lipidated ApoE4) of the present disclosure).

The DNA encoding antibodies or fragments (e.g., antigen binding fragments) thereof may also be modified, for example, by substituting the coding sequence for human heavy chain constant domain and light chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein (e.g., anti-ApoE4 antibodies of the present disclosure or fragments thereof) may be monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, such as Fab fragments, can be accomplished using routine techniques known in the art.

Chimeric or hybrid anti-ApoE4 antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

7.3. Humanized Antibodies

Anti-ApoE4 antibodies of the present disclosure or antibody fragments thereof further include humanized antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

CDR-grafted antibodies are antibodies that include the CDRs from a non-human “donor” antibody linked to the framework region from a human “recipient” antibody. Generally, CDR-grafted antibodies include more human antibody sequences than chimeric antibodies because they include both constant region sequences and variable region (framework) sequences from human antibodies. Thus, for example, a CDR-grafted humanized antibody of the disclosure can comprise a heavy chain that comprises a contiguous amino acid sequence (e.g., about 5 or more, 10 or more, or even 15 or more contiguous amino acid residues) from the framework region of a human antibody (e.g., FR-1, FR-2, or FR-3 of a human antibody) or, optionally, most or all of the entire framework region of a human antibody. CDR-grafted antibodies and methods for making them are described in, Jones et al., Nature, 321: 522-525 (1986), Riechmann et al., Nature, 0.332: 323-327 (1988), and Verhoeyen et al., Science, 239: 1534-1536 (1988)). Methods that can be used to produce humanized antibodies also are described in U.S. Pat. Nos. 4,816,567, 5,721,367, 5,837,243, and 6,180,377. CDR-grafted antibodies are considered less likely than chimeric antibodies to induce an immune reaction against non-human antibody portions. However, it has been reported that framework sequences from the donor antibodies may be required for the binding affinity and/or specificity of the donor antibody, presumably because these framework sequences affect the folding of the antigen-binding portion of the donor antibody. Therefore, when donor, non-human CDR sequences are grafted onto unaltered human framework sequences, the resulting CDR-grafted antibody can exhibit, in some cases, loss of binding avidity relative to the original non-human donor antibody. See, e.g., Riechmann et al., Nature, 332: 323-327 (1988), and Verhoeyen et al., Science, 239: 1534-1536 (1988).

In some embodiments, recovery of binding avidity can be achieved by “de-humanizing” a CDR-grafted antibody. De-humanizing can include restoring residues from the donor antibody's framework regions to the CDR grafted antibody, thereby restoring proper folding. Similar “de-humanization” can be achieved by (i) including portions of the “donor” framework region in the “recipient” antibody or (ii) grafting portions of the “donor” antibody framework region into the recipient antibody (along with the grafted donor CDRs).

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Furthermore, it is important that antibodies be humanized with retention of high affinity for the antigen and other favorable biological properties. To achieve this goal, according to one method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., lipidated ApoE4 proteins of the present disclosure), is achieved. In general, the CDR residues are directly and most substantially involved in influencing antigen binding.

In some embodiments, HUMAN ENGINEERED™ antibodies include for example “veneered” antibodies and antibodies prepared using HUMAN ENGINEERING™ technology (see for example, U.S. Pat. Nos. 5,766,886 and 5,869,619). HUMAN ENGINEERING™ involves altering an non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding and/or antigen-binding properties. Generally, low risk positions in a non-human antibody are substituted with human residues; high risk positions are rarely substituted, and humanizing substitutions at moderate risk positions are sometimes made, although not indiscriminately. Positions with prolines in the non-human antibody variable region sequence are usually classified as at least moderate risk positions. The particular human amino acid residue to be substituted at a given low or moderate risk position of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making HUMAN ENGINEERED™ proteins are described in Studnicka et al., Prot. Eng., 7: 805-814 (1994), U.S. Pat. Nos. 5,766,886, 5,770,196, 5,821,123, and 5,869,619.

“Veneered” antibodies are non-human or humanized (e.g., chimeric or CDR-grafted antibodies) antibodies that have been engineered to replace certain solvent-exposed amino acid residues so as to further reduce their immunogenicity or enhance their function. As surface residues of a chimeric antibody are presumed to be less likely to affect proper antibody folding and more likely to elicit an immune reaction, veneering of a chimeric antibody can include, for instance, identifying solvent-exposed residues in the non-human framework region of a chimeric antibody and replacing at least one of them with the corresponding surface residues from a human framework region. Veneering can be accomplished by any suitable engineering technique, including the use of the above-described HUMAN ENGINEERING™ technology.

Various forms of the humanized anti-ApoE4 antibody are contemplated. For example, the humanized anti-ApoE4 antibody may be an antibody fragment, such as an Fab, which is optionally conjugated with one or more ApoE4 ligand. Alternatively, the humanized anti-ApoE4 antibody may be an intact antibody, such as an intact IgG1 antibody.

7.4. Human Antibodies

Alternatively, human anti-ApoE4 antibodies can be generated. For example, it is possible to produce transgenic animals (e.g., mice) that are capable, upon immunization, of producing a full repertoire of human antibodies in the absence of endogenous immunoglobulin production. The homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Nat'l Acad. Sci. USA, 90:2551 (1993); Jakobovits et al., Nature, 362:255-258 (1993); Bruggermann et al., Year in Immunol., 7:33 (1993); U.S. Pat. No. 5,591,669 and WO 97/17852.

Alternatively, phage display technology can be used to produce human ApoE4 antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also U.S. Pat. Nos. 5,565,332 and 5,573,905. Additionally, yeast display technology can be used to produce human anti-ApoE4 antibodies and antibody fragments in vitro (e.g., WO 2009/036379; WO 2010/105256; WO 2012/009568; US 2009/0181855; US 2010/0056386; and Feldhaus and Siegel (2004) J. Immunological Methods 290:69-80). In other embodiments, ribosome display technology can be used to produce human anti-ApoE4 antibodies and antibody fragments in vitro (e.g., Roberts and Szostak (1997) Proc Natl Acad Sci 94:12297-12302; Schaffitzel et al. (1999) J. Immunological Methods 231:119-135; Lipovsek and Plückthun (2004) J. Immunological Methods 290:51-67).

The disclosure contemplates a method for producing an ApoE4 binding (e.g., ApoE4-specific) antibody or antigen-binding fragment (e.g., portion) thereof comprising the steps of synthesizing a library of human antibodies on phage, screening the library with an ApoE4 protein or a portion thereof, isolating phage that bind the target antigen ApoE4, and obtaining the antibody from the phage. By way of example, one method for preparing the library of antibodies for use in phage display techniques comprises the steps of immunizing a non-human animal comprising human immunoglobulin loci with target antigen or an antigenic portion thereof to create an immune response, extracting antibody producing cells from the immunized animal; isolating RNA from the extracted cells, reverse transcribing the RNA to produce cDNA, amplifying the cDNA using a primer, and inserting the cDNA into a phage display vector such that antibodies are expressed on the phage. Recombinant ApoE4 binding (e.g., ApoE4-specific) antibodies of the invention may be obtained in this way.

Phage-display processes mimic immune selection through the display of antibody repertoires on the surface of filamentous bacteriophage, and subsequent selection of phage by their binding to an antigen of choice. One such technique is described in WO 99/10494, which describes the isolation of high affinity and functional agonistic antibodies for MPL and msk receptors using such an approach. Antibodies of the disclosure can be isolated by screening of a recombinant combinatorial antibody library, preferably a scFv phage display library, prepared using human V_{L} and V_{H} cDNAs prepared from mRNA derived from human lymphocytes. Methodologies for preparing and screening such libraries are known in the art (see e.g., U.S. Pat. No. 5,969,108). There are commercially available kits for generating phage display libraries (e.g., the Pharmacia Recombinant Phage Antibody System, catalog no. 27-9400-01; and the Stratagene SurfZAP™ phage display kit, catalog no. 240612). There are also other methods and reagents that can be used in generating and screening antibody display libraries (see, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum. Antibod. Hybridomas 3:81-85; Huse et al. (1989) Science 246:1275-1281; McCafferty et al., Nature (1990) 348:552-554; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982.

In one embodiment, to isolate human antibodies that bind (e.g., specific for) the target antigen with the desired characteristics, a human V_{H} and V_{L} library are screened to select for antibody fragments having the desired specificity. The antibody libraries used in this method are preferably scFv libraries prepared and screened as described herein and in the art (McCafferty et al., PCT Publication No. WO 92/01047, McCafferty et al., (Nature 348:552-554, 1990); and Griffiths et al., (EMBO J. 12:725-734, 1993). The scFv antibody libraries preferably are screened using an ApoE4 target protein (e.g., lipidated ApoE4 protein, lipoprotein particle comprising an ApoE4 protein) as the antigen.

Alternatively, the Fd fragment (V_{H}-C_{H1}) and light chain (V_{L}-C_{L}) of antibodies are separately cloned by PCR and recombined randomly in combinatorial phage display libraries, which can then be selected for binding to a particular antigen. The Fab fragments are expressed on the phage surface, i.e., physically linked to the genes that encode them. Thus, selection of Fab by antigen binding co-selects for the Fab encoding sequences, which can be amplified subsequently. Through several rounds of antigen binding and re-amplification, a procedure termed panning, Fab that bind (e.g., specific for) the antigen are enriched and finally isolated.

The techniques of Cole et al., and Boerner et al., are also available for the preparation of human anti-ApoE4 monoclonal antibodies (Cole et al., Monoclonal Antibodies and Cancer Therapy, Alan R. Liss, p. 77 (1985) and Boerner et al., J. Immunol. 147(1): 86-95 (1991). Similarly, human anti-ApoE4 antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806, 5,569,825, 5,625,126, 5,633,425, 5,661,016 and in the following scientific publications: Marks et al., Bio/Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (1996) and Lonberg and Huszar, Intern. Rev. Immunol. 13: 65-93 (1995).

Finally, human anti-ApoE4 antibodies may also be generated in vitro by activated B-cells (see e.g., U.S. Pat. Nos. 5,567,610 and 5,229,275).

7.5. Antibody Fragments

In certain embodiments there are advantages to using anti-ApoE4 antibody fragments, rather than whole anti-ApoE4 antibodies. In some embodiments, smaller antigen-binding fragment sizes allow for rapid clearance and better brain penetration.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host cells, for example, using nucleic acids encoding anti-ApoE4 antibodies of the present disclosure. Fab, Fv and scFv antibody fragments can all be expressed in and secreted from E. coli, thus allowing the straightforward production of large amounts of these fragments. Anti-ApoE4 antibody fragments can also be isolated from the antibody phage libraries as discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host-cell culture. Production of Fab and F(ab′)2 antibody fragments with increased in vivo half-lives are described in U.S. Pat. No. 5,869,046. In other embodiments, the antibody of choice is a single chain Fv fragment (scFv). See WO 93/16185; U.S. Pat. Nos. 5,571,894 and 5,587,458. The anti-ApoE4 antibody fragment may also be a “linear antibody,” e.g., as described in U.S. Pat. No. 5,641,870. Such linear antibody fragments may be monospecific or bispecific.

### 8. Bispecific and Polyspecific Antibodies

Bispecific antibodies (BsAbs) are antibodies that have binding specificities for at least two different epitopes, including those on the same or another protein (e.g., one or more ApoE4 proteins of the present disclosure). Alternatively, one part of a BsAb can be armed to bind to the target ApoE4 antigen, and another can be combined with an arm that binds to a second protein. Such antibodies can be derived from full-length antibodies or antibody fragments (e.g., F(ab′)2 bispecific antibodies).

Methods for making bispecific antibodies are known in the art. Traditional production of full-length bispecific antibodies is based on the co-expression of two immunoglobulin heavy chain/light chain pairs, where the two chains have different specificities. Millstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO 1, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only half of the bispecific molecules provides for an easy way of separation. This approach is disclosed in WO 94/04690. For further details of generating bispecific antibodies, see, for example, Suresh et al., Methods in Enzymology 121: 210 (1986).

According to another approach described in WO 96/27011 or U.S. Pat. No. 5,731,168, the interface between a pair of antibody molecules can be engineered to maximize the percentage of heterodimers which are recovered from recombinant-cell culture. The preferred interface comprises at least a part of the C_{H}3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T-cells, as well as trigger the lytic activity of human cytotoxic lymphocytes against human breast tumor targets.

Various techniques for making and isolating bispecific antibody fragments directly from recombinant-cell culture have also been described. For example, bispecific heterodimers have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Nat'l Acad. Sci. USA, 90: 6444-6448 (1993) has provided an alternative mechanism for making bispecific antibody fragments. The fragments comprise a heavy chain variable domain (V_{H}) connected to a light chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two specificities are also contemplated. For example, trispecific antibodies can be prepared. Tutt et al., J. Immunol. 147:60 (1991).

Exemplary bispecific antibodies may bind to two different epitopes on a given molecule (e.g., an ApoE4 protein of the present disclosure). In some embodiments a bispecific antibody binds to a first antigen, such as an ApoE4 protein of the present disclosure, and a second antigen facilitating transport across the blood-brain barrier. Numerous antigens are known in the art that facilitate transport across the blood-brain barrier (see, e.g., Gabathuler R., Approaches to transport therapeutic drugs across the blood-brain barrier to treat brain diseases, Neurobiol. Dis. 37 (2010) 48-57). Such second antigens include, for example, transferrin receptor (TR), insulin receptor (HIR), TMEM30A receptor, α(2,3)-siaglycoprotein receptor, insulin-like growth factor receptor (IGFR), low-density lipoprotein receptor related proteins 1 and 2 (LPR-1 and 2), diphtheria toxin receptor, including CRM197 (a non-toxic mutant of diphtheria toxin), llama single domain antibodies such as TMEM 30(A) (Flippase), protein transduction domains such as TAT, Syn-B, or penetratin, poly-arginine or generally positively charged peptides, Angiopep peptides such as ANG1005 (see, e.g., Gabathuler, 2010), and other cell surface proteins that are enriched on blood-brain barrier endothelial cells (see, e.g., Daneman et al., PLoS One. 2010 Oct. 29; 5(10):e13741). In some embodiments, second antigens for an anti-ApoE4 antibody may include, for example, a DAP12 antigen. In other embodiments, bispecific antibodies that bind to ApoE4 may inhibit one or more ApoE4 activities.

8.1. Multivalent Antibodies

A multivalent antibody may be internalized (and/or catabolized) faster than a bivalent antibody by a cell expressing an antigen to which the antibodies bind. The anti-ApoE4 antibodies of the present disclosure or antibody fragments thereof can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises an Fc region or a hinge region. In this scenario, the antibody will comprise an Fc region and three or more antigen binding sites amino-terminal to the Fc region. The preferred multivalent antibody herein contains three to about eight, but preferably four, antigen binding sites. The multivalent antibody contains at least one polypeptide chain (and preferably two polypeptide chains), wherein the polypeptide chain or chains comprise two or more variable domains. For instance, the polypeptide chain or chains may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}—C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

8.2. Alternative Scaffolds

The alternative scaffolds provided herein may be made by any suitable method, including the illustrative methods described herein or those known in the art. For example, methods of preparing Adnectins™ are described in Emanuel et al., mAbs, 2011, 3:38-48, incorporated by reference in its entirety. Methods of preparing iMabs are described in U.S. Pat. Pub. No. 2003/0215914, incorporated by reference in its entirety. Methods of preparing Anticalins® are described in Vogt and Skerra, Chem. Biochem., 2004, 5:191-199, incorporated by reference in its entirety. Methods of preparing Kunitz domains are described in Wagner et al., Biochem. & Biophys. Res. Comm., 1992, 186:118-1145, incorporated by reference in its entirety. Methods of preparing thioredoxin peptide aptamers are provided in Geyer and Brent, Meth. Enzymol., 2000, 328:171-208, incorporated by reference in its entirety. Methods of preparing Affibodies are provided in Fernandez, Curr. Opinion in Biotech., 2004, 15:364-373, incorporated by reference in its entirety. Methods of preparing DARPins are provided in Zahnd et al., J. Mol. Biol., 2007, 369:1015-1028, incorporated by reference in its entirety. Methods of preparing Affilins are provided in Ebersbach et al., J. Mol. Biol., 2007, 372:172-185, incorporated by reference in its entirety. Methods of preparing Tetranectins are provided in Graversen et al., J. Biol. Chem., 2000, 275:37390-37396, incorporated by reference in its entirety. Methods of preparing Avimers are provided in Silverman et al., Nature Biotech., 2005, 23:1556-1561, incorporated by reference in its entirety. Methods of preparing Fynomers are provided in Silacci et al., J. Biol. Chem., 2014, 289:14392-14398, incorporated by reference in its entirety.

Further information on alternative scaffolds is provided in Binz et al., Nat. Biotechnol., 2005 23:1257-1268; and Skerra, Current Opin. in Biotech., 2007 18:295-304, each of which is incorporated by reference in its entirety.

8.3. Effector Function Engineering

It may also be desirable to modify an anti-ApoE4 ABP of the present disclosure to modify effector function and/or to increase serum half-life of the ABP. For example, the Fc receptor binding site on the constant region may be modified or mutated to remove or reduce binding affinity to certain Fc receptors, such as FcγRI, FcγRII, and/or FcγRIII. In some embodiments, the effector function is impaired by removing N-glycosylation of the Fc region (e.g., in the CH 2 domain of IgG) of an antibody. In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000).

To increase the serum half-life of the ABP, one may incorporate a salvage receptor binding epitope into the ABP (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

8.4. Affinity Maturation

It may be desirable to improve (e.g., increase) the affinity of an ABP or antigen binding fragment of the present disclosure for its target antigen, through one or more sequence alterations (e.g., in one or more HVRs/CDRs). Affinity-matured ABPs may have nanomolar, sub-nanomolar, picomolar or even sub-picomolar affinities for the target antigen, with affinity for the target antigen improved at least 2-fold, 3-fold, 4-fold, 5-fold, 10-fold, 50-fold, 100-fold, 500-fold, or 1000-fold or more, compared to a parent ABP that does not possess the sequence alteration(s). The present disclosure contemplates that affinity maturation may be used to increase the binding affinity and/or specificity for an ApoE4 target protein (e.g., lipidated ApoE4 target protein) or lipoprotein particle comprising an ApoE4 protein. In certain embodiments, affinity maturation may be used to change the relative affinities for binding to a lipidated ApoE4 target protein and a non-lipidated ApoE4 target protein (e.g., lipidated ApoE4 binding affinity at least 2-fold great than non-lipidated ApoE4 binding affinity, lipidated ApoE4 binding affinity at least 5-fold greater than non-lipidated ApoE4 protein binding affinity, lipidated ApoE4 binding affinity at least 10-fold great than non-lipidated ApoE4 binding affinity, lipidated ApoE4 binding affinity at least 100-fold greater (or more) than non-lipidated ApoE4 binding affinity, and the like).

Affinity-matured ABPs are produced by various procedures known in the art. For example, Marks et al., Bio/Technology 10:779-783 (1992) describes affinity maturation by V_{H}- and V_{L}-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992). Other affinity maturation methods include, for example, using panning (see e.g., Huls et al. (Cancer Immunol Immunother. 50:163-71 (2001)); phage display technologies (see e.g., Daugherty et al., Proc Natl Acad Sci USA. 97:2029-34 (2000)); look-through mutagenesis (see e.g., Rajpal et al., Proc Natl Acad Sci USA. 102:8466-71 (2005)); error-prone PCR (see e.g., Zaccolo et al., J. Mol. Biol. 285:775-783 (1999)); DNA shuffling (see e.g., U.S. Pat. Nos. 6,605,449 and 6,489,145, WO 02/092780 and Stemmer, Proc. Natl. Acad. Sci. USA, 91:10747-51 (1994)); alanine scanning mutagenesis (see e.g., Cunningham and Wells, (Science 244:1081-1085 (1989)); and a variety of other techniques known in the art (see e.g., WO2009/088933; WO2009/088928; WO2009/088924; Clackson et al., Nature 352:624-628, 1991; Virnekas et al., Nucleic Acids Res. 22:5600-5607, 1994; Glaser et al., J. Immunol. 149:3903-3913, 1992; Jackson et al., J. Immunol. 154:3310-3319, 1995; Schier et al., J. Mol. Biol. 255:28-43, 1996; and Yang et al., J. Mol. Biol. 254:392-403, 1995), incorporated by reference herein in their entirety.

8.5. Other Amino Acid Sequence Modifications

Amino acid sequence modifications of anti-ApoE4 ABPs of the present disclosure, or ABP fragments thereof, are also contemplated. For example, it may be desirable to improve the binding affinity and/or other biological properties of the ABPs. Amino acid sequence variants of the ABPs are prepared by introducing appropriate nucleotide changes into the nucleic acid encoding the ABPs, or by peptide synthesis. Such modifications include, for example, deletions from, and/or insertions into and/or substitutions of, residues within the amino acid sequences of the ABP. Any combination of deletion, insertion, and substitution is made to arrive at the final construct, provided that the final construct possesses the desired characteristics (i.e., the ability to bind or physically interact with an ApoE4 protein of the present disclosure). The amino acid changes also may alter post-translational processes of the ABP, such as changing the number or position of glycosylation sites.

A useful method for identification of certain residues or regions of the anti-ApoE4 ABP that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the target antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed ABP variants are screened for the desired activity.

Amino acid sequence insertions include amino-(“N”) and/or carboxy-(“C”) terminal fusions ranging in length from one residue to polypeptides containing a hundred or more residues, as well as intrasequence insertions of single or multiple amino acid residues. Examples of terminal insertions include an ABP with an N-terminal methionyl residue or the ABP fused to a cytotoxic polypeptide. Other insertional variants of the ABP molecule include the fusion to the N- or C-terminus of the ABP to an enzyme or a polypeptide which increases the serum half-life of the ABP.

Another type of variant is an amino acid substitution variant. These variants have at least one amino acid residue in the ABP molecule replaced by a different residue. The sites of greatest interest for substitutional mutagenesis include the hypervariable regions, but FR alterations are also contemplated. Conservative substitutions are shown in the Table A below under the heading of “preferred substitutions.” If such substitutions result in a change in biological activity, then more substantial changes, denominated “exemplary substitutions” in Table 2, or as further described below in reference to amino acid classes, may be introduced and the products screened.

TABLE 2
Amino Acid Substitutions

| Original | | |
|---|---|---|
| Residue | Exemplary Substitutions | Preferred Substitutions |
| Ala (A) | val; leu; ile | Val |
| Arg (R) | lys; gln; asn | Lys |
| Asn (N) | gln; his; asp, lys; arg | Gln |
| Asp (D) | glu; asn | Glu |
| Cys (C) | ser; ala | Ser |
| Gln (Q) | asn; glu | Asn |
| Glu (E) | asp; gln | Asp |
| Gly (G) | Ala | Ala |
| His (H) | asn; gln; lys; arg | Arg |
| Ile (I) | leu; val; met; ala; phe; norleucine | Leu |
| Leu (L) | norleucine; ile; val; met; ala; phe | Ile |
| Lys (K) | arg; gln; asn | Arg |
| Met (M) | leu; phe; ile | Leu |
| Phe (F) | leu; val; ile; ala; tyr | Tyr |
| Pro (P) | Ala | Ala |
| Ser (S) | Thr | Thr |
| Thr (T) | Ser | Ser |
| Trp (W) | tyr; phe | Tyr |
| Tyr (Y) | trp; phe; thr; ser | Phe |
| Val (V) | ile; leu; met; phe; ala; norleucine | Leu |

Substantial modifications in the biological properties of the ABP are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions entail exchanging a member of one of these classes for another class.

Any cysteine residue not involved in maintaining the proper conformation of the ABP also may be substituted, generally with serine, to improve the oxidative stability of the molecule and prevent aberrant crosslinking. Conversely, cysteine bond(s) may be added to the ABP to improve its stability (for example, where the ABP is an antibody fragment, such as an Fv fragment).

A preferred type of substitutional variant involves substituting one or more hypervariable region residues of a parent antibody (e.g., a humanized or human anti-ApoE4 antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen (e.g., an ApoE4 protein of the present disclosure). Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the ABP alters the original glycosylation pattern of the ABP. By altering is meant deleting one or more carbohydrate moieties found in the ABP, and/or adding one or more glycosylation sites that are not present in the ABP.

Glycosylation of ABPs is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the ABP is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original ABP (for O-linked glycosylation sites).

Nucleic acid molecules encoding amino acid sequence variants of the anti-ApoE4 ABP are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the ABPs (e.g., anti-ApoE4 ABPs of the present disclosure) or ABP fragments.

8.6. Other ABP Modifications

Anti-ApoE4 ABPs of the present disclosure, or ABP fragments thereof, can be further modified to contain additional non-proteinaceous moieties that are known in the art and readily available. Preferably, the moieties suitable for derivatization of the ABP are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the ABP may vary, and if more than one polymer is attached, they can be the same or different molecules. In general, the number and/or type of polymers used for derivatization can be determined based on considerations including, but not limited to, the particular properties or functions of the ABP to be improved, whether the ABP derivative will be used in a therapy under defined conditions. Such techniques and other suitable formulations are disclosed in Remington: The Science and Practice of Pharmacy, 20th Ed., Alfonso Gennaro, Ed., Philadelphia College of Pharmacy and Science (2000).

### 9. Nucleic Acids, Vectors and Host Cells

Anti-ApoE4 ABPs of the present disclosure may be produced using recombinant methods and compositions, such as for example isolated nucleic acids encoding anti-ApoE4 antibodies, e.g., as described in U.S. Pat. No. 4,816,567. In some embodiments, isolated nucleic acids (e.g., nucleic acid molecules) comprising a nucleotide sequence encoding any of the anti-ApoE4 ABPs or antigen binding fragments of the present disclosure are provided. Such nucleic acids may comprise a nucleotide sequence that encodes an amino acid sequence containing the V_{L} and/or an amino acid sequence containing the V_{H} of the anti-ApoE4 antibody (e.g., the light and/or heavy chains of the antibody). Additionally, such nucleic acids may comprise a nucleotide sequence that encodes an amino acid sequence containing the light chain variable region and/or an amino acid sequence containing the heavy chain variable region of the anti-ApoE4 antibody. In some embodiments, one or more vectors (e.g., expression vectors) comprising such nucleic acids are provided. In some embodiments, a host cell comprising such nucleic acids or vectors (e.g., expression vectors) is also provided. In some embodiments, the host cell comprises (e.g., has been transfected with, has been transduced with): (1) a vector containing a nucleic acid that encodes an amino acid sequence containing the V_{L} of the antibody and an amino acid sequence containing the V_{H} of the antibody, or (2) a first vector containing a nucleic acid that encodes an amino acid sequence containing the V_{L} of the antibody and a second vector containing a nucleic acid that encodes an amino acid sequence containing the V_{H} of the antibody. In some embodiments, the host cell comprises (e.g., has been transfected with, has been transduced with) a nucleic acid molecule encoding a heavy chain variable region of the antibody and a nucleic acid molecule encoding a light chain variable region of the antibody, wherein the heavy chain and light chain variable regions are expressed by different nucleic acid molecules or from the same nucleic acid molecule. In some embodiments, the host cell is eukaryotic, e.g., a Chinese Hamster Ovary (CHO) cell or lymphoid cell (e.g., Y0, NS0, Sp20 cell).

Methods of making an anti-ApoE4 ABP of the present disclosure are provided. In some embodiments, the method includes culturing a host cell of the present disclosure containing a nucleic acid encoding the anti-ApoE4 ABP, under conditions suitable for expression of the ABP. In some embodiments, the ABP is subsequently recovered from the host cell (or host cell culture medium).

For recombinant production of an anti-ApoE4 ABP or antigen binding fragment of the present disclosure, a nucleic acid encoding the anti-ApoE4 ABP or fragments thereof is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the anti-ApoE4 ABPs of the present disclosure, fragments thereof, or polypeptides (including antibodies) described herein include, for example, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Stratagene, and Invitrogen.

Expression vectors generally are replicable polynucleotide constructs that contain a nucleic acid of the present disclosure (e.g., nucleic acid operably linked to an expression control element). The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator); other expression control elements. For expression (i.e., translation), one or more translational controlling elements are also usually required, such as ribosome binding sites, translation initiation sites, and stop codons.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-ApoE4 ABP of the present disclosure. In some embodiments, the expression vector contains a nucleic acid molecule comprising a nucleotide sequence encoding any of the anti-ApoE4 ABPs or antigen binding fragments of the present disclosure operably linked to an expression control element.

Suitable host cells for cloning or expression of ABP-encoding vectors include prokaryotic or eukaryotic cells. For example, anti-ApoE4 ABPs of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, N.J., 2003), pp. 245-254, describing expression of antibody fragments in E. coli.). After expression, the ABP may be isolated from the bacterial cell paste in a soluble fraction and can be further purified.

In addition to prokaryotes, eukaryotic microorganisms, such as filamentous fungi or yeast, are also suitable cloning or expression hosts for ABP-encoding vectors, including fungi and yeast strains whose glycosylation pathways have been “humanized,” resulting in the production of an ABP with a partially or fully human glycosylation pattern (e.g., Gerngross, Nat. Biotech. 22:1409-1414 (2004); and Li et al., Nat. Biotech. 24:210-215 (2006)).

Suitable host cells for the expression of glycosylated ABP can also be derived from multicellular organisms (invertebrates and vertebrates). Examples of invertebrate cells include plant and insect cells. Numerous baculoviral strains have been identified which may be used in conjunction with insect cells, for example for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing ABPs in transgenic plants.).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for ABP production, see, e.g., Yazaki and Wu, Methods in Molecular Biology, Vol. 248 (B. K. C. Lo, ed., Humana Press, Totowa, N.J.), pp. 255-268 (2003).

### 10. Pharmaceutical Compositions

Anti-ApoE4 ABPs of the present disclosure can be incorporated into a variety of formulations for therapeutic administration by combining the ABPs with appropriate pharmaceutically acceptable carriers or diluents, and may be formulated into preparations in solid, semi-solid, liquid or gaseous forms. Examples of such formulations include, for example, tablets, capsules, powders, granules, ointments, solutions, suppositories, injections, inhalants, gels, microspheres, and aerosols. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, for example, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

A pharmaceutical composition of the present disclosure can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include, for example, sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The concentration of ABP in these formulations can vary widely, for example from less than about 0.5%, usually at or at least about 1% to as much as 15 or 20% by weight and will be selected, for example, based on fluid volumes, viscosities, and other characteristics of the formulation, in accordance with the particular mode of administration selected. For example, a pharmaceutical composition for parenteral injection could be made up to contain 1 ml sterile buffered water, and 50 mg of ABP, and a composition for intravenous infusion could be made up to contain 250 ml of sterile Ringer's solution, and 150 mg of ABP. Actual methods for preparing parenterally administrable compositions will be known or apparent to those skilled in the art and are described in more detail in, for example, Remington's Pharmaceutical Science, 15th ed., Mack Publishing Company, Easton, Pa. (1980).

The ABPs of the present disclosure can be lyophilized for storage and reconstituted in a suitable carrier prior to use. This technique has been shown to be effective with conventional immunoglobulins. Any suitable lyophilization and reconstitution techniques can be employed. It will be appreciated by those skilled in the art that lyophilization and reconstitution can lead to varying degrees of ABP activity loss and that use levels may have to be adjusted to compensate.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, such as endotoxins, which may be present during the synthesis or purification process. Compositions for parenteral administration are also sterile, substantially isotonic and made under GMP conditions.

Formulations may be optimized for retention and stabilization in the brain or central nervous system. When the agent is administered into the cranial compartment, it is desirable for the agent to be retained in the compartment, and not to diffuse or otherwise cross the blood-brain barrier. Stabilization techniques include cross-linking, multimerizing, or linking to groups such as polyethylene glycol, polyacrylamide, neutral protein carriers, and the like. in order to achieve an increase in molecular weight.

Other strategies for increasing retention include the entrapment of the ABP, such as an anti-ApoE4 ABP of the present disclosure, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e., having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, such as carboxymethylcellulose esters characterized by being water insoluble, a molecular weight of about 5 kD to 500 kD. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### 11. Pharmaceutical Dosages

Pharmaceutical compositions of the present disclosure containing an anti-ApoE4 ABP of the present disclosure may be administered to an individual in need of treatment with the anti-ApoE4 ABP, preferably a human, in accord with known methods, such as intravenous administration as a bolus or by continuous infusion over a period of time, by intramuscular, intraperitoneal, intracerobrospinal, intracranial, intraarterial cerebral infusion, intracerebroventricular, intraspinal, subcutaneous, intra-articular, intrasynovial, intrathecal, oral, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the anti-ApoE4 ABPs of the present disclosure, normal dosage amounts may vary from about 10 ng/kg up to about 100 mg/kg of an individual's body weight or more per day, preferably about 0.1 mg/kg/day to 10 mg/kg/day, depending upon the route of administration. For repeated administrations over several days or longer, depending on the severity of the disease, disorder, or condition to be treated, the treatment is sustained until a desired suppression of symptoms is achieved.

An exemplary dosing regimen may include administering an initial dose of an anti-ApoE4 ABP, of about 2 mg/kg, followed by a maintenance dose of about 1 mg/kg every other week. Other dosage regimens may be useful, depending on the pattern of pharmacokinetic decay that the physician wishes to achieve. For example, dosing an individual from one to twenty-one times a week is contemplated. In certain embodiments, dosing ranging from about 3 μg/kg to about 2 mg/kg (such as about 3 μg/kg, about 10 μg/kg, about 30 μg/kg, about 0.1 mg/kg, about 0.3 mg/kg, about 1 mg/kg, about 2 mg/kg, and about 5 mg/kg) may be used.

It may be advantageous to administer the ABP or binding fragment as a fixed dose, independent of a dose per subject weight ratio. In some embodiments, the ABP or fragment is administered as a fixed dose of about 500 mg, about 250 mg, about 100 mg, about 50 mg, about 25 mg, about 10 mg, or about 5 mg.

In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the anti-ApoE4 ABP administered, can vary over time independently of the dose used.

Dosages for a particular anti-ApoE4 ABP may be determined empirically in individuals who have been given one or more administrations of the anti-ApoE4 ABP. Individuals are given incremental doses of an anti-ApoE4 ABP. To assess efficacy of an anti-ApoE4 ABP, a clinical symptom of any of the diseases, disorders, or conditions of the present disclosure (e.g., dementia, Alzheimer's disease, cerebral amyloid angiopathy, cardiovascular disease, coronary heart disease, age-related macular degeneration, peripheral vascular disease, hypertriglyceridemia, hyperlipoproteinemia Type III, multiple sclerosis, or a traumatic or non-traumatic acquired brain injury) can be monitored.

Administration of an anti-ApoE4 ABP of the present disclosure can be continuous or intermittent, depending, for example, on the recipient's physiological condition, whether the purpose of the administration is therapeutic or prophylactic, and other factors known to skilled practitioners. The administration of an anti-ApoE4 ABP may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the invention that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### 12. Therapeutic Uses

Further aspects of the present disclosure provide a method of preventing, treating or reducing the risk of a disease, condition or disorder associated with ApoE4 expression in a subject, comprising administering to the subject a therapeutically effective amount of an anti-ApoE4 ABP, such as for example, an anti-ApoE4 ABP or pharmaceutical composition described herein.

In some embodiments, the subject is an ε4 homozygote. In some embodiments, a subject is an ε4 heterozygote. In some embodiments, the heterozygote is an ε4/ε3 heterozygote. In some embodiments, the heterozygote is an ε4/ε2 heterozygote. In some embodiments, the subject carries a natural variant of ApoE4 such as, for example, L→P in isoform E4 Freiburg (residue 28), R→H in isoform E4 P.D. (residue 274), or S→R (residue 296).

In some embodiments, the disease, condition or disorder is a dementia, Alzheimer's disease, cerebral amyloid angiopathy, cardiovascular disease, coronary heart disease, age-related macular degeneration, peripheral vascular disease, hypertriglyceridemia, hyperlipoproteinemia Type III, multiple sclerosis, or a traumatic or non-traumatic acquired brain injury, such as for example, head trauma, cerebral hemorrhage, stroke or epilepsy. In some embodiments, the subject is an ApoE4 carrier.

Yet further aspects of the present disclosure provide methods of modulating one or more functions or, or phenotypes associated with, an ApoE4 protein or a lipoprotein particle comprising an ApoE4 protein in a subject, comprising administering to the subject a therapeutically effective amount of an anti-ApoE4 ABP, such as for example, an anti-ApoE4 ABP or pharmaceutical composition described herein. In some embodiments, the one or more functions of, or phenotypes associated with, an ApoE4 protein or a lipoprotein particle comprising an ApoE4 protein are selected from among the functions or phenotypes provided in Table 1.

In some embodiments, the methods of treatment provided herein further comprise the administration of one or more additional therapeutic agents. In some embodiments, the one or more additional therapeutic agents is selected from an amyloid beta directed therapeutic, a tau protein directed therapeutic, and combinations thereof. In certain embodiments, the one or more additional therapeutic agents is selected from an antibody that binds a CD33 protein, an antibody that binds a sortilin protein, an antibody that binds a TREM2 protein, an antibody that binds an amyloid beta protein, an antibody that binds tau protein, a BACE inhibitor, a gamma secretase inhibitor, an agent that disaggregates amyloid beta oligomers, an agent that disaggregates tau fibrils, and combinations thereof. In some embodiments, the additional therapeutic agent is a DAP12 targeted therapy.

12.1. Dementia

Dementia is a non-specific syndrome (i.e., a set of signs and symptoms) that presents as a serious loss of global cognitive ability in a previously unimpaired person, beyond what might be expected from normal aging. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, for example, memory, attention span, language, and problem solving. Generally, symptoms must be present for at least six months to before an individual is diagnosed with dementia.

Exemplary forms of dementia include, for example, frontotemporal dementia, Alzheimer's disease dementia, vascular dementia, semantic dementia, and dementia with Lewy bodies.

In certain embodiments, provided herein is a method of treating, preventing, or reducing the risk of dementia in a subject in need thereof, comprising administering an effective amount of an anti-ApoE4 ABP provided herein. In some embodiments, administering an anti-ApoE4 ABP of the present disclosure may modulate one or more functions of, or phenotypes associated with, an ApoE4 protein or a lipoprotein particle comprising an ApoE4 protein in a subject having dementia (e.g., one or more functions or phenotypes provided in Table 1).

12.2. Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

In certain embodiments, provided herein is a method of treating, preventing, or reducing the risk of Alzheimer's disease in a subject in need thereof, comprising administering an effective amount of an anti-ApoE4 ABP provided herein. In some embodiments, administering an anti-ApoE4 ABP of the present disclosure may modulate one or more functions of, or phenotypes associated with, an ApoE4 protein or a lipoprotein particle comprising an ApoE4 protein in a subject having Alzheimer's disease (e.g., one or more functions or phenotypes provided in Table 1).

12.3. Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include, for example, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoff's phenomenon, which is an exacerbation of extant symptoms due to an exposure to higher than usual ambient temperatures; and Lhermitte's sign, which is an electrical sensation that runs down the back when bending the neck.

In certain embodiments, provided herein is a method of treating, preventing, or reducing the risk of multiple sclerosis in a subject in need thereof, comprising administering an effective amount of an anti-ApoE4 ABP provided herein. In some embodiments, administering an anti-ApoE4 ABP of the present disclosure may modulate one or more functions of, or phenotypes associated with, an ApoE4 protein or a lipoprotein particle comprising an ApoE4 protein in a subject having multiple sclerosis (e.g., one or more functions or phenotypes provided in Table 1).

The invention will be more fully understood by reference to the following Examples. They should not, however, be construed as limiting the scope of the invention.

### 13. Selected Embodiments

Provided below are selected non-limiting embodiments of the ABPs provided herein and methods of their use and manufacture:

1. An isolated antigen-binding protein (ABP) that specifically binds to a lipidated ApoE4 protein
2. The ABP of embodiment 1, wherein the lipidated ApoE4 protein is associated with a lipoprotein particle.
3. The ABP of any of the preceding embodiments, wherein the affinity of the ABP for the lipidated ApoE4 protein, as measured by K_{d}, is greater than the affinity of the ABP for non-lipidated ApoE4 protein.
4. The ABP of any of the preceding embodiments, wherein the ABP does not bind non-lipidated ApoE4 protein.
5. The ABP of any of the preceding embodiments, wherein the affinity of the ABP for the lipidated ApoE4 protein, as measured by K_{d}, is at least 3-fold greater than the affinity of the ABP for non-lipidated ApoE4 protein.
6. The ABP of any of the preceding embodiments, wherein the affinity of the ABP for the lipidated ApoE4 protein, as measured by K_{d}, is 10⁻⁶ or less, 10⁻⁷ or less, 10⁻⁸ or less, 10⁻⁹ or less, 10¹⁰ or less, or 10⁻¹¹ M or less.
7. The ABP of any of the preceding embodiments, wherein the ApoE4 protein is a human protein.
8. The ABP of any of the preceding embodiments, wherein the ApoE4 protein is a wild-type protein or a naturally occurring variant.
9. The ABP of any of embodiments Error! Reference source not found.-Error! Reference source not found., wherein the lipoprotein particle is selected from a chylomicron, a high density lipoprotein (HDL) particle, an intermediate density lipoprotein (IDL) particle, a low density lipoprotein (LDL) particle, and a very low density lipoprotein (VLDL) particle.
10. The ABP of any of embodiments Error! Reference source not found.-Error! Reference source not found., wherein the lipoprotein particle comprises at least one lipoprotein other than an ApoE4 protein.
11. The ABP of any of the preceding embodiments, wherein the ABP binds to one or more amino acid residues within an ApoE4 epitope selected from:

| | |
|---|---|
| (a) | amino acid residues 55-78 |
| | (QVTQELRALMDETMKELKAYKSEL (i.e., SEQ ID NO: 2)) |
| | of SEQ ID NO: 1; |
| (b) | amino acid residues 134-150 |
| | (RVRLASHLRKLRKRLLR (i.e., SEQ ID NO: 3)) |
| | of SEQ ID NO: 1; |
| (c) | amino acid residues 154-158 |
| | (DLQKR (i.e., SEQ ID NO: 4)) of SEQ ID NO: 1; |
| (d) | amino acid residues 208-272 |
| | (QAWGERLRARMEEMGSRTRDRLDEVKEQVAEVRAKLEEQAQQIRLQ |
| | AEAFQARLKSWFEPLVEDM (i.e., SEQ ID NO: 5)) |
| | of SEQ ID NO: 1; |
| (e) | amino acid residues 225-299 |
| | (TRDRLDEVKEQVAEVRAKLEEQAQQIRLQAEAFQARLKSWFEPLVE |
| | DMQRQWAGLVEKVQAAVGTSAAPVPSDNH (i.e., SEQ ID NO: |
| | 6)) of SEQ ID NO: 1; and |
| (f) | amino acid residues 244-272 |
| | (EEQAQQIRLQAEAFQARLKSWFEPLVEDM (i.e., SEQ ID |
| | NO: 7)) of SEQ ID NO: 1. |

12. The ABP of any of embodiments 1-Error! Reference source not found., wherein the ABP binds to an ApoE4 epitope comprising at least one of amino acid residues Arg-61, Glu-109, Arg-112, Arg-136, His-140, Lys-143, Arg-150, Asp-154, Arg-158, Arg-172, and Glu-255.
13. The ABP of any of the preceding embodiments, wherein the ABP disrupts the interaction between an N-terminal domain and C-terminal domain of an ApoE4 protein.
14. The ABP of embodiment 4, wherein the ABP disrupts the interaction between ApoE4 helix 2, comprising amino acid residues 55-78 (QVTQELRALMDETMKELKAYKSEL (i.e., SEQ ID NO: 2)) of SEQ ID NO: 1, and the ApoE4 lipid binding domain, comprising amino acid residues 244-272 (EEQAQQIRLQAEAFQARLKSWFEPLVEDM (i.e., SEQ ID NO: 7)) of SEQ ID NO: 1.
15. The ABP of any of embodiments 4 or 5, wherein the ABP disrupts the interaction between amino acid residues Arg-61 and Glu-255 of SEQ ID NO: 1.
16. The ABP of any of the preceding embodiments, wherein the ABP modulates a function of, or phenotype associated with, ApoE4 or a lipoprotein particle comprising ApoE4.
17. The ABP of embodiment Error! Reference source not found., wherein the function of, or phenotype associated with, ApoE4 or lipoprotein particle comprising ApoE4 is modulated so that said function or phenotype more closely resembles the corresponding function of, or phenotype associated with, ApoE2 or a lipoprotein particle comprising ApoE2.
18. The ABP of embodiment Error! Reference source not found., wherein the function of, or phenotype associated with, ApoE4 or lipoprotein particle comprising ApoE4 is modulated so that said function or phenotype more closely resembles the corresponding function of, or phenotype associated with, ApoE3 or a lipoprotein particle comprising ApoE3.
19. The ABP of any of embodiments Error! Reference source not found.-Error! Reference source not found., wherein the function or phenotype is selected from phospholipid-rich particle binding; triglyceride-rich particle binding; LDLR binding; LDLR family member binding; HSPG binding; processing of APP to amyloid beta, BBB leakage; formation of neurofibrillary tangles; inflammation; production of amyloid beta; clearance of amyloid beta from the CNS by transport across the BBB; accumulation of amyloid beta in tissue; level of intraneuronal amyloid beta; internalization of amyloid beta into nerve cells; binding and stabilization of amyloid beta; LDL cholesterol levels; clinically undesirable lipid profile; LDLR levels on cell surfaces; LDLR protein family member levels on cell surfaces; recovery from traumatic or non-traumatic acquired brain injury; rate of aging; cognitive impairment; phagocytosis in microglia, macrophages, monocytes or astrocytes; uptake of soluble amyloid beta by astrocytes; myelin cholesterol levels; adverse reaction or poor responsiveness to statin therapy; risk of developing Alzheimer's disease or late-onset Alzheimer's disease, or symptoms or pathology thereof; risk of developing cardiovascular disease, or symptoms or pathology thereof; risk of developing dementia, or symptoms or pathology thereof; risk of developing cerebral amyloid angiopathy, or symptoms or pathology thereof; risk of developing multiple sclerosis, or symptoms or pathology thereof; risk of developing age-related macular degeneration, or symptoms or pathology thereof; pathological Alzheimer's disease-like gene expression profile; glucose metabolism in pre-symptomatic Alzheimer's disease brain; volume of brain structures in pre-symptomatic Alzheimer's disease brain; senile plaque formation; uptake of amyloid beta by neurons, astroglia, microglia, oligodendrocytes or endothelial cells; pathological microglial activity; competition with soluble amyloid beta for LRP1-dependent uptake by astrocytes; clearance of apoptotic neurons, nerve tissue debris; non-nerve tissue debris, bacteria, foreign bodies, or disease-associated proteins or peptides; hypercholesterimia; and combinations thereof.
20. The ABP of any of the preceding embodiments, wherein the ABP has one or more activities, in vitro or in a subject, selected from:
(a) increasing binding of lipidated ApoE4 to a phospholipid-rich particle;
(b) reducing binding of lipidated ApoE4 to a triglyceride rich lipid particle;
(c) increasing the release of ApoE4 from a triglyceride-rich lipid particle;
(d) reducing the binding of lipidated ApoE4 to LDLR;
(e) reducing the binding of lipidated ApoE4 to an LDLR family member;
(f) increasing binding of ApoE4 to HSPG;
(g) reducing ApoE4-associated processing of APP to amyloid beta;
(h) reducing ApoE4-associated inhibition of amyloid beta clearance;
(i) reducing ApoE4-associated BBB leakage;
(j) reduces ApoE4-associated formation of neurofibrillary tangles;
(k) reducing ApoE4-associated inflammation;
(l) reducing ApoE4-associated production of amyloid beta;
(m) reducing ApoE4-associated reduction in clearance of amyloid beta across the BBB, or increasing clearance of amyloid beta across the BBB;
(n) reducing ApoE4-associated accumulation of amyloid beta in tissue, or increasing clearance of amyloid beta from a tissue;
(o) reducing ApoE4-associated intraneuronal accumulation of amyloid beta;
(p) reducing ApoE4-associated internalization of amyloid beta into nerve cells;
(q) reducing ApoE4-associated stabilization of amyloid beta and the formation of amyloid beta multimers;
(r) reducing ApoE4-associated increase in LDL cholesterol levels;
(s) reducing ApoE4-associated clinically undesirable lipid profiles;
(t) reducing ApoE4-associated downregulation of LDLR on cell surfaces;
(u) reducing ApoE4-associated downregulation of LDLR protein family members on cell surfaces;
(v) reducing ApoE4-associated delayed recovery from traumatic or non-traumatic acquired brain injury;
(w) reducing ApoE4-associated risk of developing Alzheimer's disease or late onset Alzheimer's disease, or symptoms or pathology thereof;
(x) reducing ApoE4-associated risk of developing cardiovascular disease or symptoms or pathology thereof;
(y) reducing ApoE4-associated risk of developing dementia or symptoms or pathology thereof;
(z) reducing ApoE4-associated risk of developing cerebral amyloid angiopathy or symptoms or pathology thereof;
(aa) reducing ApoE4-associated risk of developing multiple sclerosis or symptoms or pathology thereof;
(bb) reducing ApoE4-associated risk of developing age-related macular degeneration or symptoms or pathology thereof;
(cc) reducing ApoE4-associated acceleration of aging;
(dd) reducing or delaying ApoE4-associated cognitive impairment, or normalizing cognitive function in a subject expressing ApoE4;
(ee) reducing ApoE4-associated inhibition of phagocytosis in microglia, macrophages, monocytes, or astrocytes;
(ff) reducing ApoE4-associated decrease in soluble amyloid beta uptake by astrocytes;
(gg) reducing ApoE4-associated depletion of myelin cholesterol;
(hh) reducing ApoE4-associated adverse drug reaction to statin therapy or poor responsiveness to statin therapy;
(ii) reducing ApoE4-associated aberrant gene expression profiles associated with Alzheimer's disease;
(jj) reducing ApoE4-associated reduction in glucose metabolism in brains of pre-symptomatic Alzheimer's disease patients;
(kk) reducing ApoE4-associated reduction in volume of brain structures in pre-symptomatic Alzheimer's disease patients;
(ll) reducing ApoE4-associated senile plaque formation;
(mm) reducing ApoE4-associated decrease in amyloid beta uptake by neurons, astroglia, microglia, oligodendroglia or endothelial cells;
(nn) reducing ApoE4-associated pathological microglial activity;
(oo) reducing the binding of ApoE4 to LRP1, thereby decreasing ApoE4's ability to compete with soluble amyloid beta for binding to LRP1;
(pp) reducing ApoE4-associated reduction in clearance of apoptotic neurons, nerve tissue debris, non-nerve tissue debris, bacteria, foreign bodies, or disease-associated proteins or peptides;
(qq) and combinations thereof.
   21. The ABP of any of embodiments Error! Reference source not found.-7, wherein the phospholipid-rich particle is an HDL particle.
   22. The ABP of any of embodiments Error! Reference source not found.-8, wherein the triglyceride-rich particle is a VLDL particle.
   23. The ABP of any of embodiments Error! Reference source not found.-9, wherein the LDLR family member is selected from LDLR, VLDLR, LRP1, LRP1b, LRP2, LRP3, LRP4, LRP5, LRP6, LRP7, LRP8, LRP10, LRP11, LRP12 sortilin, TREM2, and combinations thereof.
   24. The ABP of any of embodiments Error! Reference source not found.-10, wherein the clinically undesirable lipid profile is selected from one or more of high total cholesterol (>240 mg/dL) and high LDL (>160 mg/dL).
   25. The ABP of any of embodiments Error! Reference source not found.-Error! Reference source not found., wherein the traumatic or non-traumatic brain injury is selected from head trauma, cerebral hemorrhage, stroke, epilepsy, and combinations thereof.
   26. The ABP of any of embodiments Error! Reference source not found.-Error! Reference source not found., wherein the cardiovascular disease is selected from coronary heart disease, atherosclerosis, peripheral vascular disease, and combinations thereof.
   27. The ABP of any of embodiments Error! Reference source not found.-Error! Reference source not found., wherein the dementia is selected from at least one of vascular dementia and frontotemporal dementia.
   28. The ABP of any of embodiments Error! Reference source not found.-Error! Reference source not found., wherein the pathological microglial activity is selected from increased inflammatory polarization, decreased repair function, decreased phagocytosis, and combinations thereof.
   29. The ABP of any of embodiments Error! Reference source not found.-Error! Reference source not found., wherein the disease-associated protein or peptide is selected from amyloid beta, tau, IAPP, TDP-43, alpha-synuclein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain, S-IBM, and combinations thereof.
   30. The ABP of any of embodiments Error! Reference source not found.-Error! Reference source not found., wherein the clearance of apoptotic neurons, nerve tissue debris, non-nerve tissue debris, bacteria, foreign bodies, or disease-associated proteins or peptides is by phagocytosis.
   31. The ABP of any of the preceding embodiments, wherein ApoE4 binding to atypical LDLR family members is preserved in the presence of the ABP and, optionally, wherein the atypical LDLR family member is selected from TREM2, sortilin, SORL1, SORCS1, SORCS2, SORCS, and combinations thereof.
   32. The ABP of any of embodiments Error! Reference source not found.-11, wherein the rate of aging is measured by quantifying telomere length.
   33. The ABP of any of the preceding embodiments, wherein the ABP selectively binds to lipidated ApoE4 that is bound to amyloid beta.
   34. The ABP of embodiment Error! Reference source not found., wherein the ABP enhances the clearance of ApoE4-bound amyloid beta.
   35. The ABP of any of the preceding embodiments, wherein the ABP is selected from an antibody and an alternative scaffold.
   36. The ABP of embodiment Error! Reference source not found., wherein the ABP is an antibody selected from a human antibody, a humanized antibody, a chimeric antibody, a bispecific antibody, and a multivalent antibody.
   37. The ABP of embodiment Error! Reference source not found., wherein the antibody is a monoclonal antibody.
   38. The ABP of any of embodiment Error! Reference source not found.-12, wherein the antibody is an antibody fragment.
   39. The ABP of embodiment 13, wherein the antibody fragment is selected from a Fab, Fab′, Fab′-SH, F(ab′)₂, Fv, and a scFv fragment.
   40. The ABP of embodiment Error! Reference source not found., wherein the alternative scaffold is selected from a fibronectin, a β-sandwich, a lipocalin, an EETI-II/AGRP, a BPTI/LACI-D1/ITI-D2, a thioredoxin peptide aptamer, a protein A, an ankyrin repeat, a gamma-B-crystallin/ubiquitin, a CTLD3, a FYNOMER, and an AVIMER.
   41. The ABP of any of the preceding embodiments, wherein the ABP comprises an immunoglobulin constant region.
   42. The ABP of any of the preceding embodiments, wherein the lipidated ApoE4 protein comprises an ApoE4 protein bound to a lipid selected from a triglyceride, a phospholipid, a sphingolipid, a cholesterol ester, cholesterol, DMPC, triolein, phosphatidylcholine, phosphatidylethanolamine, phosphatidylglycerol, phosphatidylserine, phosphatidylinositol, PIP, phosphatidic acid, and cardiolipin, and combinations thereof.
   43. An isolated nucleic acid molecule comprising a nucleotide sequence that encodes:
(a) a heavy chain or light chain variable region of an antibody of any one of embodiments Error! Reference source not found.-Error! Reference source not found.; or
(b) an alternative scaffold of embodiment Error! Reference source not found.
   44. A vector comprising the nucleic acid molecule of embodiment Error! Reference source not found.
   45. The vector of embodiment Error! Reference source not found., wherein the vector is an expression vector comprising an expression control element that is operably linked to the nucleic acid molecule.
   46. A host cell comprising a nucleic acid molecule of embodiment Error! Reference source not found. or an expression vector of any of embodiments Error! Reference source not found.-Error! Reference source not found.
   47. The host cell of embodiment Error! Reference source not found., comprising a nucleic acid molecule encoding a heavy chain variable region of an antibody and a nucleic acid molecule encoding a light chain variable region of an antibody, wherein the heavy chain and light chain variable regions are expressed by different vectors or from the same vector.
   48. A method of producing an ABP, comprising culturing the host cell of any of embodiments Error! Reference source not found.-Error! Reference source not found. so that the ABP is produced.
   49. The method of embodiment Error! Reference source not found., further comprising recovering the ABP produced by the host cell.
   50. A pharmaceutical composition comprising the ABP of any of the preceding embodiments and a pharmaceutically acceptable carrier.
   51. A method of preventing, treating or reducing the risk of a disease, condition or disorder in a subject that is an ApoE4 carrier, comprising administering to the subject a therapeutically effective amount of an ABP of any one of embodiments 1-Error! Reference source not found. or a pharmaceutical composition of embodiment Error! Reference source not found.
   52. The method of embodiment 14, wherein the disease, condition or disorder is selected from the group consisting of dementia, cognitive disorder, Alzheimer's disease, cerebral amyloid angiopathy, cardiovascular disease, age-related macular degeneration, multiple sclerosis, traumatic or non-traumatic acquired brain injury, adverse reaction or poor responsiveness to statin therapy, reduced glucose metabolism in the brain, reduced volume of brain structures, hypercholesterimia, lipoprotein glomerulopathy, sea-blue histiocyte disease, and combinations thereof.
   53. The method of embodiment 15, wherein the dementia is selected from one or more of frontotemporal dementia and vascular dementia.
   54. The method of any of embodiments 15-Error! Reference source not found., wherein the Alzheimer's disease is selected from late onset Alzheimer's disease, sporadic form of Alzheimer's disease, and familial Alzheimer's disease.
   55. The method of any of embodiments 15-Error! Reference source not found., wherein the cardiovascular disease is selected from coronary heart disease, atherosclerosis, peripheral vascular disease, and combinations thereof.
   56. The method of any of embodiments 15-Error! Reference source not found., wherein the traumatic or non-traumatic acquired brain injury is selected from head trauma, cerebral hemorrhage, stroke, epilepsy, and combinations thereof.
   57. The method of any of embodiments 14-Error! Reference source not found., wherein the ABP reduces, prevents or delays progression of the disease, condition or disorder.
   58. A method of modulating one or more functions of, or phenotypes associated with, an ApoE4 protein or a lipoprotein particle comprising an ApoE4 protein in a subject that is an ApoE4 carrier, comprising administering to the subject a therapeutically effective amount of an ABP of any of embodiments 1-Error! Reference source not found. or a pharmaceutical composition of embodiment Error! Reference source not found.
   59. The method of embodiment Error! Reference source not found., wherein the function of, or phenotype associated with, ApoE4 or lipoprotein particle comprising ApoE4 is modulated so that said function or phenotype more closely resembles the corresponding function of, or phenotype associated with, ApoE2 or a lipoprotein particle comprising ApoE2.
   60. The method of embodiment Error! Reference source not found., wherein the function of, or phenotype associated with, ApoE4 or lipoprotein particle comprising ApoE4 is modulated so that said function or phenotype more closely resembles the corresponding function of, or phenotype associated with, ApoE3 or a lipoprotein particle comprising ApoE3.
   61. The method of any of embodiments Error! Reference source not found.-Error! Reference source not found., wherein the function or phenotype is selected from phospholipid-rich particle binding; triglyceride-rich particle binding; LDLR binding; LDLR family member binding; HSPG binding; processing of APP to amyloid beta, BBB leakage; formation of neurofibrillary tangles; inflammation; production of amyloid beta; clearance of amyloid beta from the CNS by transport across the BBB; accumulation of amyloid beta in tissue; level of intraneuronal amyloid beta; internalization of amyloid beta into nerve cells; binding and stabilization of amyloid beta; LDL cholesterol levels; clinically undesirable lipid profile; LDLR levels on cell surfaces; LDLR protein family member levels on cell surfaces; recovery from traumatic or non-traumatic acquired brain injury; rate of aging; cognitive impairment; phagocytosis in microglia, macrophages, monocytes or astrocytes; uptake of soluble amyloid beta by astrocytes; myelin cholesterol levels; adverse reaction or poor responsiveness to statin therapy; risk of developing Alzheimer's disease or late-onset Alzheimer's disease, or symptoms or pathology thereof, risk of developing cardiovascular disease, or symptoms or pathology thereof; risk of developing dementia, or symptoms or pathology thereof; risk of developing cerebral amyloid angiopathy, or symptoms or pathology thereof; risk of developing multiple sclerosis, or symptoms or pathology thereof; risk of developing age-related macular degeneration, or symptoms or pathology thereof; pathological Alzheimer's disease-like gene expression profile; glucose metabolism in pre-symptomatic Alzheimer's disease brain; volume of brain structures in pre-symptomatic Alzheimer's disease brain; senile plaque formation; uptake of amyloid beta by neurons, astroglia, microglia, oligodendrocytes or endothelial cells; pathological microglial activity; competition with soluble amyloid beta for LRP1-dependent uptake by astrocytes; clearance of apoptotic neurons, nerve tissue debris; non-nerve tissue debris, bacteria, foreign bodies, or disease-associated proteins or peptides; and combinations thereof.
   62. The method of any of embodiments Error! Reference source not found.-Error! Reference source not found., wherein the ABP has one or more activities in the subject selected from:
(a) increasing binding of lipidated ApoE4 to a phospholipid-rich particle;
(b) reducing binding of lipidated ApoE4 to a triglyceride rich lipid particle;
(c) increasing the release of ApoE4 from a triglyceride-rich lipid particle;
(d) reducing the binding of lipidated ApoE4 to LDLR;
(e) reducing the binding of lipidated ApoE4 to an LDLR family member;
(f) increasing binding of ApoE4 to HSPG;
(g) reducing ApoE4-associated processing of APP to amyloid beta;
(h) reducing ApoE4-associated inhibition of amyloid beta clearance;
(i) reducing ApoE4-associated BBB leakage;
(j) reduces ApoE4-associated formation of neurofibrillary tangles;
(k) reducing ApoE4-associated inflammation;
(l) reducing ApoE4-associated production of amyloid beta;
(m) reducing ApoE4-associated reduction in clearance of amyloid beta across the BBB, or increasing clearance of amyloid beta across the BBB;
(n) reducing ApoE4-associated accumulation of amyloid beta in tissue, or increasing clearance of amyloid beta from a tissue;
(o) reducing ApoE4-associated intraneuronal accumulation of amyloid beta;
(p) reducing ApoE4-associated internalization of amyloid beta into nerve cells;
(q) reducing ApoE4-associated stabilization of amyloid beta and the formation of amyloid beta multimers;
(r) reducing ApoE4-associated increase in LDL cholesterol levels;
(s) reducing ApoE4-associated clinically undesirable lipid profiles;
(t) reducing ApoE4-associated downregulation of LDLR on cell surfaces;
(u) reducing ApoE4-associated downregulation of LDLR protein family members on cell surfaces;
(v) reducing ApoE4-associated delayed recovery from traumatic or non-traumatic acquired brain injury;
(w) reducing ApoE4-associated risk of developing Alzheimer's disease or late onset Alzheimer's disease, or symptoms or pathology thereof;
(x) reducing ApoE4-associated risk of developing cardiovascular disease or symptoms or pathology thereof;
(y) reducing ApoE4-associated risk of developing dementia or symptoms or pathology thereof;
(z) reducing ApoE4-associated risk of developing cerebral amyloid angiopathy or symptoms or pathology thereof;
(aa) reducing ApoE4-associated risk of developing multiple sclerosis or symptoms or pathology thereof;
(bb) reducing ApoE4-associated risk of developing age-related macular degeneration or symptoms or pathology thereof;
(cc) reducing ApoE4-associated acceleration of aging;
(dd) reducing or delaying ApoE4-associated cognitive impairment, or normalizing cognitive function in a subject expressing ApoE4;
(ee) reducing ApoE4-associated inhibition of phagocytosis in microglia, macrophages, monocytes, or astrocytes;
(ff) reducing ApoE4-associated decrease in soluble amyloid beta uptake by astrocytes;
(gg) reducing ApoE4-associated depletion of myelin cholesterol;
(hh) reducing ApoE4-associated adverse drug reaction to statin therapy or poor responsiveness to statin therapy;
(ii) reducing ApoE4-associated aberrant gene expression profiles associated with Alzheimer's disease;
(jj) reducing ApoE4-associated reduction in glucose metabolism in brains of pre-symptomatic Alzheimer's disease patients;
(kk) reducing ApoE4-associated reduction in volume of brain structures in pre-symptomatic Alzheimer's disease patients;
(ll) reducing ApoE4-associated senile plaque formation;
(mm) reducing ApoE4-associated decrease in amyloid beta uptake by neurons, astroglia, microglia, oligodendroglia or endothelial cells;
(nn) reducing ApoE4-associated pathological microglial activity;
(oo) reducing the binding of ApoE4 to LRP1, thereby decreasing ApoE4's ability to compete with soluble amyloid beta for binding to LRP 1;
(pp) reducing ApoE4-associated reduction in clearance of apoptotic neurons, nerve tissue debris, non-nerve tissue debris, bacteria, foreign bodies, or disease-associated proteins or peptides;
(qq) and combinations thereof.
   63. The method of any of embodiments 14-Error! Reference source not found., wherein the subject has a genotype selected from:
(a) an ε4 homozygote;
(b) an ε4/ε3 heterozygote; and
(c) an ε4/ε2 heterozygote.
   64. The method of any of embodiments 14-16, further comprising administering to the subject a therapeutically effective amount of one or more additional therapeutic agents.
   65. The method of embodiment 17, where in the one or more additional therapeutic agents is selected from an amyloid beta-directed therapeutic, a tau protein-directed therapeutic, an antibody that binds a CD33 protein, an antibody that binds a sortilin protein, an antibody that binds a TREM2 protein, an antibody that binds an amyloid beta protein, an antibody that binds tau protein, a BACE inhibitor, a gamma secretase inhibitor, an agent that disaggregates amyloid beta oligomers, an agent that disaggregates tau fibrils, and combinations thereof.
   66. The method of any of embodiments 14-17, wherein the ABP is administered by intravenous, intramuscular, intraperitoneal, intracerobrospinal, intracranial, intraarterial cerebral infusion, intracerebroventricular, intraspinal, subcutaneous, intra-articular, intrasynovial, intrathecal, oral, topical, or inhalation routes.

### EXAMPLES

### Example 1: ApoE Protein and ApoE Containing Lipoprotein Particles

ApoE proteins, including lipidated and non-lipidated ApoE4 and ApoE2 protein, as well as lipoprotein particles containing ApoE4 or ApoE2, for use in the Examples below are obtained from a variety of sources or using standard methods known in the art. For example, sources include commercial suppliers, such as, Recombinant Human ApoE4 Protein, ProSci, Inc., #40-138; Recombinant Human ApoE2 Protein, ProSci, Inc., #40-140; Recombinant Human ApoE4 Protein, MBL International, #JM-4699-500; Recombinant Human ApoE2 Protein, Leinco Technologies, #A215. Apolipoprotein particles containing ApoE proteins also may be isolated from ApoE2 or ApoE4 homozygote human sources, such as plasma and or cerebrospinal fluid using standard procedures know in the art, such as for example ultracentrifugation. Recombinant ApoE proteins may be prepared directly using bacterial expression systems (see e.g., (Zaiou, et al., J Lipid Res 41:1087-95 (2000))), or similarly using other expression systems, such as mammalian cells or insect cells.

More specifically, in one example, recombinant ApoE4 and ApoE2 are generated in vitro from E. coli cultures after transformation of plasmid DNA encoding the ApoE4 or ApoE2 into protease-deficient E. coli strain BL21 (DE3). An overnight culture of in Luria-Bertarni (LB) broth supplemented with ampicillin (100 g/mL) is used to inoculate a 6-L culture in LB medium. The culture is grown at 37° C. with constant shaking until its absorbance reaches 0.5 OD at 600 nm, and expression is then induced by adding IPTG to a final concentration of 0.4 mm. The expression is continued for 2.5 h, and the cells are harvested by centrifugation at 4,000 rpm for 20 min at 4° C. in a J6 rotor. The cells are resuspended in 30 mL of ice-cold extraction buffer (150 mm NaCl, 20 mm Na2HPO4, 25 mm EDTA, 2 mm phenylmethylsulfonyl fluoride, 1% Trasylol (aprotinin), 0.1% 2-mercaptoethanol, pH 7.4). The suspension is sonicated on ice with a sonifier cell disruptor 350 (Branson Ultrasonics, Danbury, Conn.) fitted with a ½-inch tip for three cycles of 1 min on and 2 min off. Bacterial debris is removed by centrifugation at 40,000 g for 20 min at 4° C. To prepare soluble proteins in the cytoplasm of the E. coli, solid GdnHCl and 2-mercaptoethanol are added to the supernatant to final concentrations of 7 M and 1%, respectively. The mixture is incubated at 4° C. overnight, insoluble material is removed by centrifugation for 10 min at 40,000 g, and the supernatant containing the recombinant proteins is recovered for further purification.

ApoE4 and ApoE2 are separated from the E. coli extracts by fast-performance liquid chromatography (FPLC) using a combination of gel-filtration, ion-exchange, and affinity techniques. First, the supernatant is applied to a Sephacryl S300 column (200×2.6 cm, 1-mL/min flow rate) previously equilibrated with a buffer containing 4 m GdnHCl, 0.1 m Tris-HCl (pH 7.4), 1 mm EDTA, and 0.1% 2-mercaptoethanol. ApoE is eluted with the same buffer, and the elution profile is determined by monitoring the absorbance of the effluent at 280 nm. Protein samples are analyzed for purity by sodium dodecyl sulfate-polyacrylamide gel electrophoresis (SDS-PAGE) using 8-25% Phast gels (Amersham Pharmacia Biotech, Piscataway, N.J.) at each stage of the procedure. Fractions (12.5 mL) containing ApoE are pooled and extensively dialyzed against 20 mm NH4HCO3. After dialysis, the protein samples are lyophilized and solubilized in 0.1 m NH4HCO3, pH 7.4. ApoE is then applied to a 5×16 cm Q-Sepharose ion-exchange column equilibrated with 50 mL of buffer A (6 m urea, 20 mm Tris-HCl, pH 7.4). Bound ApoE is eluted by applying a 0-1 M NaCl gradient (buffer B: 6 m urea, 1 m NaCl, 20 mm Tris-HCl, pH 7.4). The fractions containing ApoE are pooled, dialyzed against 25 mm NH4HCO3, pH 8.0, and passed through three heparin columns (HiTrap (1.5×1.6 cm); Amersham Pharmacia Biotech). The column is washed with 25 mm NH4HCO3, pH 8.0, to remove unbound proteins, and the ApoE protein is eluted with 750 mm NH4HCO3, pH 8.0.

Preparation of ApoE.DMPC (dimyristoylphosphatidylcholine) lipid complexes can be made as follows. ApoE4 or ApoE2 proteins are mixed with DMPC vesicles at a ratio of 1:3.75 (protein-DMPC, by weight) and isolated by KBr density gradient ultracentrifugation as described previously. Briefly, the desired amount of DMPC is dried from a chloroform-methanol solution under nitrogen in a 15-mL tube. The residue is redissolved in 1-2 mL of benzene, frozen, and lyophilized. Lipids are sonicated in a buffer containing 0.15 m NaCl, 10 mm disodium EDTA, and 1 mm Tris-HCl, pH 7.6. The slightly translucent solution of DMPC vesicles is then centrifuged at low speed and kept at room temperature. The appropriate amount of ApoE dissolved in 0.1 m NH4HCO3, pH 8.1, is added to the tube in the presence of 2-mercaptoethanol (at 0.5· 1/100·g of protein), and the mixture is recycled three times through the gel-liquid crystal transition temperature of the DMPC (23.5° C.) by warming to 37° C. and cooling on ice, taking 15 min for each cycle. The DMPC.apoE complexes are separated from uncomplexed protein and lipid by density gradient centrifugation. A linear KBr salt gradient (d 1.006-1.21 g/mL) is prepared in polyallomer tubes (Beckman Instruments). The lipid-protein complexes are layered on top of the gradient and centrifuged in an SW-55 rotor at 15° C. for 20 h (369,000 g). The majority of the lipid-protein complex is removed from collected fractions in the density range of 1.09-1.10 g/mL. These fractions are pooled and dialyzed against saline-EDTA and stored at 4° C. The apoE-DMPC discoidal complexes are sized by negative-stain electron microscopy with a JEOL (Tokyo, Japan) 100CXII electron microscope.

### Example 2: Isolation of Anti-ApoE4 Antibodies from Display Libraries

### Phage Panning and Rescue

Lipidated human ApoE4 or lipoprotein particles containing human ApoE4 are biotinylated with Sulfo-NHS-LC-Biotin (Pierce, Rockford, Ill.) using the manufacturer's protocol and 16-fold molar excess of biotin reagent. The biotinylation of is confirmed by surface plasmon resonance (SPR). For the first round of phage panning, 10¹¹ cfu of phage particles from an scFv phage display library are blocked for 1 h at room temperature (RT) in 1 ml of 5% milk/PBS with gentle rotation. Blocked phage are twice deselected for 30 minutes against streptavidin-coated magnetic Dynabeads® M-280 (Invitrogen Dynal AS, Oslo, Norway).

The biotin-ApoE4 protein or lipoprotein particle solution is incubated with blocked streptavidin-coated magnetic Dynabeads® M-280 (Invitrogen Dynal AS, Oslo, Norway) for 30 minutes with gentle rotation in order to immobilize the biotin-ApoE4. The deselected phage are incubated with the biotin-streptavidin beads for 2 h at RT. The beads are washed. For the first round of panning, beads are quickly washed (i.e., beads are pulled out of solution using a magnet and resuspended in 1 ml wash buffer) three times with PBS-0.1% TWEEN, followed by three times with PBS. For the second round of panning, beads are quickly washed five times with PBS-0.1% TWEEN followed by a one 5 minute wash (in 1 ml wash buffer at room temperature with gentle rotation) with PBS-0.1% TWEEN and then five times with PBS followed by one 5 minute wash with PBS. For the third round of panning, beads are quickly washed four times with PBS-0.1% TWEEN, followed by two washes for five minutes with PBS-0.1% TWEEN and then four quick washes with PBS, followed by two 5 minute washes with PBS.

The ApoE4-bound phage are eluted with 100 mM triethylamine (TEA) (30 min incubation at RT) which is then neutralized with 1M Tris-HCl (pH 7.4). The eluted phage are used to infect TG1 bacterial cells (Stratagene, Calif.) when they reach an OD₆₀₀ of about 0.5. Following infection for 30 min at 37° C. without shaking, and for 30 min at 37° C. with shaking at 90 rpm, cells are pelleted and resuspended in 2YT media supplemented with 100 ug/ml ampicillin and 2% glucose. The resuspended cells are plated on 2YT agar plates with 100 ug/ml carbenicillin and 2% glucose and incubated overnight at 30° C.

Phage is then rescued with helper phage VCSM13 (New England Biolabs, MA) at a multiplicity of infection (MOI) about 10. Following helper phage infection at an OD₆₀₀ of 0.6 at 37° C. for 30 min without rotation and 30 min incubation at 37° C. at 150 rpm, cell pellets are resuspended in 2YT media supplemented with 100 ug/ml ampicillin and 50 ug/ml kanamycin and allowed to grow overnight at 30° C. Phage in the supernatant are recovered after rigorous centrifugation and used for the next round of panning. In order to monitor the enrichment resulting from the phage selections, the amount of input and output phage is titered for each round of panning.

### Gene III Excision and Generation of Bacterial Periplasmic Extracts

Before screening the phage panning output scFv clones for binding to the ApoE4, the gene III gene is first excised from the phagemid vectors to enable production of secreted scFv. In order to do this, a plasmid midi prep (Qiagen, Valencia, Calif.) of the final panning round output pool of clones is digested with the restriction enzyme. The digestion product without the gene III is then allowed to self-ligate with T4 DNA ligase (New England Biolabs, MA) and used to transform chemically-competent TOP10 E. coli cells (Invitrogen, Carlsbad, Calif.). Individual transformed colonies in 96-well plates are then used to generate bacterial periplasmic extracts according to standard methods, with a 1:3 volume ratio of ice-cold PPB solution (Teknova, Hollister, Calif.) and double distilled water (ddH₂O) and two protease inhibitor cocktail tablets (Roche, Ind.). The lysate supernatants are assayed by ELISA, as described below.

### ELISA Screening of Antibody Clones on Lipidated ApoE4 or Apolipoprotein Particles

ELISA Maxisorp® plates (Thermo Fisher Scientific, Rochester, N.Y.) are coated overnight at 4° C. with 3 ug/ml ApoE4 in PBS. Plates are then blocked for 1 h at RT with 400 ul/well 5% milk/PBS. Bacterial periplasmic extracts are also blocked with 5% milk/PBS for 1 h and then added to the coated ELISA plate (50 ul/well) and allowed to bind to ApoE4 on the ELISA plate for 2 h at RT. Bound scFv fragments are detected with murine anti-c-myc mAb (Roche, Ind.) for 1 h at RT followed by goat anti-mouse HRP-conjugated antisera (Thermo Scientific, Rockford, Ill.). Three washes with PBS-0.1% TWEEN-20 (Teknova, Hollister, Calif.) are performed following every stage of the ELISA screens. Color is developed at 450 nm absorbance with 50 ul/well soluble 3.3′,5.5′-tetramethylbenzidine (TMB) substrate (EMD chemicals, Calbiochem, NJ) and stopped with 1M H₂SO₄ (50 ul/well) Antibody Phage Display: Methods and Protocols. (Methods in Molecular Biology Springer Protocols, Humana Press; 2nd ed. 2009 edition. Robert Aitken editor)

### Yeast Display Libraries

Yeast display libraries, such as those in which antibodies are displayed as a fusion with the Aga2p protein on the surface of yeast, may be used in a similar manner with biotinylated antigen. Further isolations of yeast that express the desired ApoE4 binding antibodies are performed by flow cytometry with fluorescent labeled lipidated APOE4. Yeast that is bound to the fluorescent lipidated APOE4 through the antibody on its cell surface can be isolated and separated from non-bound yeast by flow cytometry. The same yeast can then be exposed subsequently to fluorescent non-lipidated ApoE4 to identify and isolate antibodies which preferentially bind to lipidated ApoE4 (Gera, et al., Methods 60:15-26 (2013))

### Example 3: ApoE Variants Display Differential Binding to LDL Family Receptors

Antibodies that modify or modulate the binding of lipidated ApoE4 to exhibit greater similarity (e.g., more closely resemble) that of lipidated ApoE2 and/or ApoE3 are identified in vitro, for example using recombinant lipidated ApoE4, using purified lipidated ApoE4 from plasma of ApoE4 human carriers, or from cell cultures or transgenic animals expressing the human ApoE4. The ability of ApoE4, in complex with lipids, to bind the different receptors (e.g., LDL family of receptors) is tested in the absence or presence of ApoE4 antibodies. Among various antibodies of the present disclosure, in some embodiments, anti-ApoE4 antibodies that reduce or prevent binding to LDLR but not to VLDLR and/or LRP1 are identified.

### Example 4: Preparation of Lipidated ApoE4

Binding of lipidated ApoE4 to the LDL receptor (LDLR) in the presence or absence of antibodies of the present disclosure is quantified in vitro, in cell cultures. ApoE4 is tested as a lipidated form with a single lipid, such as DMPC, or as VLDL-like emulsions as detailed below, or other lipid emulsions (Dong, et al., J Lipid Res 39:1173-80 (1998)). ApoE4 is mixed with DMPC at a ratio of 1:3.75 (w:w, protein:DMPC), and phospholipid:protein complexes are isolated by density gradient ultracentrifugation. The VLDL-like emulsion particles are prepared as described previously. Briefly, triolein (100 mg) and egg yolk phosphatidylcholine (25 mg) (Sigma) are mixed and then dried under a stream of nitrogen. After resuspension in 5 ml of 10 mm Tris-Cl buffer (pH 8.0) containing 0.1 m KCl and 1 mm EDTA, the materials are sonicated as previously described. The mean size of the emulsion particles prepared by this procedure is expected to be similar to that of the native VLDL, based on previously published studies. The emulsion particles are incubated with ApoE4 at 37° C. for 2 h. Particle-bound ApoE4 is separated from unbound ApoE4 on a Superose 6 column (Pharmacia Fine Chemicals).

### Example 5: Binding of Lipidated ApoE4 to LDLR on Cell Surfaces

One week before the experiment, normal human fibroblasts that express the LDL receptor are plated at 3.5×10⁴ cells/dish. On day 5, the cells are switched to medium containing 10% lipoprotein-deficient serum. On day 7, the cells are incubated in medium containing 2.0 g/ml of ¹²⁵I-labeled LDL and various concentrations of ApoE4-DMPC. The ability of ApoE4-DMPC to displace the binding of ¹²⁵I-labeled LDL to the LDLR on cells in the presence or absence of antibodies of the present disclosure is determined at 4° C. In another example, binding is quantified in a solid-phase assay in a cell free system (Dong, et al., J Lipid Res 39:1173-80 (1998)). Binding is performed using an N-terminal 22 kD fragment of lipidated ApoE4, but similar studies can be performed with the full-length form. Lipidated recombinant ApoE4 or a 22-kDa fragment are isolated as previously described, and receptor-binding activity is determined with a solid-phase assay in the presence or absence of antibodies described herein. The fragments (100 ng/well) in phosphate-buffered saline (150 mm NaCl, 20 mm sodium phosphate, pH 7.4) (PBS) are incubated overnight at 4° C. in 96-well microtiter plates (Dynatech Immunlon, Chantilly, Va.). After each subsequent step, the plates are washed with 1% bovine serum albumin (BSA) in PBS. Non-specific binding is blocked with 4% BSA in PBS for 1 h at room temperature. The soluble LDL receptor fragment is diluted to approximately 10 ng/ml in 2 mM phosphate and 0.15 m NaCl (pH 7.2) containing 3% BSA and 20 mm CaCl₂ and incubated in the 22-kDa ApoE-coated wells for 2 h at room temperature. Bound receptor is detected with the anti-LDL receptor monoclonal antibody C7 (Amersham), followed by horseradish peroxidase-labeled anti-mouse immunoglobulin G (IgG) (Amersham) and color development with O-phenylenediamine dihydrochloride (Sigma) according to the manufacturer's instructions. Determinations are performed in triplicate in three separate plates. In parallel, wells without added receptor, an anti-ApoE4 antibody is used for detection to ensure that the microtiter wells are coated with comparable amounts of each ApoE4. The binding of lipidated ApoE2 is determined as a control.

In another example, interaction between lipidated ApoE4 and LDLR and related receptors is determined by surface plasmon resonance (SPR), by pull-down assays, by NMR, or by X-ray crystallography, in the presence or absence of antibodies of the present disclosure. Binding is quantified to LDLR or related receptors of ApoE, including VLDL receptor, LRP1, LRP2, APOER2/LRP8, MEGF7, LDLR-related protein 1, LDLR-related protein 1b, Megalin, Sortilin, SORLA (Nykjaer, et al., Trends Cell Biol 12:273-80 [2002]), and other receptors of this family in the presence or absence of antibodies of the present disclosure: This can be performed with lipidated or non-lipidated proteins, as well as ApoE4 containing lipoproprotein particles, in the presence or absence of antibodies of the present disclosure.

Whereas the lipidation state is a major determinant of binding to LDLR, it is not a major determinant of binding to LRP1 or VLDLR (Ruiz, et al., J Lipid Res 46:1721-31 [2005]). Lipidation can be performed using a variety of techniques, or lipidated forms can be isolated from cell cultures or from human plasma or from plasma of transgenic animals (see e.g., above Examples). The ability of lipidated ApoE4 to bind VLDLR and LRP in the absence or presence of antibodies of the present disclosure can also be determined as follows (Ruiz, et al., J Lipid Res 46:1721-31 [2005]). The soluble VLDL receptor fragment containing ligand binding repeats 1-8 (sVLDLr1-8) is prepared and characterized as described. In some experiments, a soluble form of the human VLDL receptor, termed sVLDLr, that contains the entire ectodomain is used. This receptor is prepared using the Drosophila expression system (Invitrogen) with the inducible/secreted kit according to the manufacturer's protocol. The secreted sVLDLr is purified by first removing Cu2 ions from the media by passage over a Chelex-100 (Bio-Rad) column and then by affinity chromatography over receptor-associated protein (RAP)-Sepharose as described.

Soluble forms of the LDL receptor are prepared in E. coli. LRP is purified from human placenta, whereas RAP is expressed in E. coli and prepared as described. ApoE2, ApoE3, and ApoE4 are prepared as described. Because of the presence of cysteine in ApoE2 and ApoE3, they are prone to form intermolecular disulfide-linked forms that are visualized by SDS-PAGE under nonreducing conditions. When present, the disulfide-linked aggregates are removed by dialyzing the protein into 20 mM HEPES, 150 mM NaCl, pH 7.4 (HBS buffer) containing 20 mM DTT for 1 h at room temperature, followed by dialysis overnight against nitrogenated HBS buffer. SDS-PAGE under nonreducing conditions and fast-protein liquid chromatography analysis are used to confirm that ApoE preparations re free of disulfidelinked structures after treatment. ApoE monoclonal antibodies 3H1 and 1D7 have been described, as well as mouse monoclonal anti-VLDL receptor antibodies 5F3, 1H5, and 1H10, which are generated by immunizing VLDL receptor knockout mice with recombinant sVLDLr1-8 and prepared as described. Antibodies are purified using protein G-Sepharose (Amersham Pharmacia Biotech). Purified mouse IgGs from Sigma-Aldrich, Inc. (St. Louis, Mo.) are used as controls for mouse anti-VLDL receptor antibodies. For assays involving cells, IgG samples are heat-inactivated for 30 min at 50° C. before use. BSA is purchased from Sigma-Aldrich, Inc.

The ability of lipidated ApoE4 to bind VLDLR or related receptors is evaluated by coating microtiter wells with sVLDLr1-8, or LRP. After coating and blocking with BSA, the wells are incubated with 5 nM ¹²⁵I-labeled apoE4 in the absence or presence of monoclonal antibodies of the present invention. After repeated washing using standard procedures, radioactivity is quantified in a gamma-counter.

### Example 6: Binding of Lipidated ApoE4 to Receptor in Solid Phase Binding Assays

Another method to determine binding of lipidated ApoE4 to the various receptors in the presence or absence of antibodies of the present disclosure is to immobilize lipidated ApoE4 on microtiter wells (IMMULON 2HB plates from Fisher Scientific) (Ruiz, et al., J Lipid Res 46:1721-31 [2005]) at a concentration of 4 g/ml. The microtiter wells are then blocked with 3% BSA. LRP and sVLDLr1-8 are added, in the presence or absence of the antibodies and binding is allowed to occur for 16 h at 4° C. After binding, wells are washed three times. Bound LRP is detected with monoclonal antibody 11H4, and bound sVLDLr1-8 is detected with mouse polyclonal antibodies against sVLDLr1-8. To determine specificity, the binding of LRP and sVLDLr1-8 to BSA-coated wells is also measured. Bound monoclonal antibodies are detected with anti-mouse IgG-alkaline phosphatase-conjugated antibodies (Bio-Rad). After incubation with phosphatase substrate (Sigma number 104) in 0.1 M glycine, 1 mM MgCl 2, and 1 mM ZnCl 2, pH 10.4, the absorbance for each sample is measured at 405 nm. Data are analyzed by nonlinear regression analysis using SigmaPlot.

To measure the binding of monoclonal antibodies to the VLDL receptor, sVLDLr1-8 is first immobilized onto microtiter wells. After blocking with BSA, increasing amounts of antibodies are added. After binding and washing, bound monoclonal antibodies are detected with anti-mouse IgG-alkaline phosphatase-conjugated antibodies (Bio-Rad). After incubation with phosphatase substrate (Sigma number 104) in 0.1M glycine, 1 mM MgCl₂, and 1 mM ZnCl₂, pH 10.4, the absorbance for each sample is measured at 405 nm. Data are analyzed using nonlinear regression analysis using SigmaPlot.

### Example 7: Surface Plasmon Resonance Measurements of ApoE4 Binding to Receptor

To evaluate the affinity of lipidated ApoE4 for receptors, such as VLDLR and LRP, in the presence or absence of antibodies of the present disclosure, surface plasmon resonance (SPR) can be used with a BIAcore 3000 biosensor (BIAcore AB, Uppsala, Sweden)(Ruiz, et al., J Lipid Res 46:1721-31 [2005]). Purified sVLDL1-8 and LRP are immobilized onto a CM5 sensor chip surface at densities of 3.5 mol/mm 2 (120 resonance units (RU)) and 5.8 fmol/mm 2 (3,500 RU), respectively, by amine coupling in accordance with the manufacturer's instructions (BIAcore AB). One flow cell is activated and blocked with 1 M ethanolamine without any protein and is used as a control surface to normalize SPR signal from receptors immobilized with flow cells. Most binding experiments are conducted in standard HBS-P buffer, pH 7.4 (BIAcore AB), containing 0.005% Tween 20 at a flow rate of 30 l/min and temperature of 25° C. Some direct binding experiments with the LRP and sVLDLr1-8 immobilized receptors are carried out in the presence of 2 mM CaCl₂ in HBS-P buffer at a flow rate of 10 l/min. Sensor chip surfaces are regenerated by 30 s pulses of 100 mM H₃PO₄. All injections use the Application Wizard in the automated method. Data are analyzed with BIA evaluation 3.0 software (BIAcore AB) using the equilibrium analysis model. The maximum change in response units (Rmax) from this analysis is replotted versus ApoE4 concentration in the presence or absence of antibodies, and the data are fit to a single class of sites by nonlinear regression analysis using SigmaPlot 9.0 software. To measure the binding of ApoE4 to the VLDL receptor in the presence or absence of antibodies of the present disclosure, 100 nM of each protein is injected directly over the CM5 chip surface in which sVLDLr is immobilized at a density of 3,000 RU. As a control for the experiment, a flow cell with immobilized ovalbumin (500 RU) is used. All injections are done in KINJECT mode, and Rmax reflects the SPR response of ApoE protein binding to the VLDL receptor.

Lipidated ApoE4 also is thought to bind to atypical LDLR family members, such as Sortilin, SORCS1, and SORLA—the latter being implicated in Alzheimer's disease risk (Carlo, et al., J Neurosci 33:358-70 [2013]). Binding of lipidated ApoE4 to these atypical LDLR family members, or fragments of the extracellular components of these, and modification of binding in the presence of antibodies to the binding levels of lipidated ApoE2 or 4, is assessed using SPR or other binding assays as above for LDLR.

### Example 8: Distribution of Lipidated ApoE4 to HDL and VLDL

Another screen for antibodies of the present disclosure involves testing their ability to modulate (e.g., change) the distribution properties of lipidated ApoE4 to lipoprotein particles, such that the antibody bound ApoE4 exhibits greater similarity to (e.g., mimics) the distribution properties of APOE2 or ApoE3. Lipidated ApoE4 generally exhibits greater (e.g., increased) distribution to VLDL (or chylomicrons or other less dense particles), and lesser (e.g., reduced) binding or distribution to HDL and other more dense particles. Furthermore, ApoE4 generally interacts more avidly with lipids compared to ApoE2 or ApoE3. Antibodies that alter the ApoE4 profile to more closely resemble an ApoE2 profile of lipid and lipoprotein binding may be useful therapeutically.

ApoE distribution among plasma lipoproteins in the presence or absence of antibodies of the present disclosure can be determined in vitro or in vivo (Dong, et al., J Lipid Res 39:1173-80 [1998]). In one in vitro example, ApoE4 is iodinated with the Bolton-Hunter reagent (Dupont NEN). The iodinated protein is reduced with b-mercaptoethanol (0.1% final concentration) and incubated with normal human plasma at 37° C. for 2 h as described previously. The plasma is fractionated into various lipoprotein classes by Superose 6 column chromatography (10/50 HR, Pharmacia). The column is eluted with 20 mm phosphate buffer (pH 7.4) containing 150 mm NaCl at a flow rate of 0.5 ml/min, and 0.5-ml fractions are collected. The ¹²⁵I content is determined in a Beckman 8000 counter (Beckman Instruments). Partitioning of ApoE4 into dense (HDL-like) and less dense (VLDL-like) particles in the presence or absence of function changing antibodies can thus be determined.

In another example, recombinant ApoE isoforms are evaluated for interaction with artificial liposomal particles resembling VLDL or HDL (Nguyen, et al., Biochemistry 49:10881-9 [2010]) in the presence or absence of antibodies of the present disclosure and antibodies that elicit interactions between ApoE4 and liposomal particles that mimic those of ApoE2 or ApoE3 are identified. Human ApoE4 is expressed in E. coli as thioredoxin fusion proteins and isolated and purified as described. Full length ApoE3 and ApoE4 (residues 1-299), their 22 kDa N-terminal fragments (residues 1-191) and 12 kDa C-terminal fragment (residues 192-299), as well as the C-terminal truncated forms (1-260, 1-272) have been described previously. The ApoE preparations are at least 95% pure as assessed by SDS-PAGE. The ApoE variants are ¹⁴C-trace labeled by reductive methylation as described previously. In all experiments, the ApoE sample is freshly dialyzed from 6M GdnHCl and 10 mM DTT solution into a buffer solution before use. ApoE concentrations are determined either by a measurement of the absorbance at 280 nm or by the Lowry procedure. HDL3 and VLDL are purified by sequential ultracentrifugation from a pool of normolipidemic human plasma as described. Dimyristoyl phosphatidylcholine (DMPC) is obtained from Avanti Polar Lipids (Pelham, Ala.) and egg yolk phosphatidylcholine (PC) and triolein are purchased from Sigma (St. Louis, Mo.). 8-Anilino-1-napthalenesulfonic acid (ANS) is purchased from Molecular Probes (Eugene, Oreg.).

### Example 9: Distribution of Lipidated ApoE4 to Emulsion Particles Resembling HDL and VLDL

Emulsion particles are prepared by sonication of a triolein/egg yolk PC mixture (3.5/lw/w) in pH 7.4 Tris buffer. The binding of ApoE4 in the presence or absence of antibodies of the present disclosure is monitored by incubating ¹⁴C-labeled ApoE4 protein with emulsion for 1 h at room temperature and separating free and bound ApoE4 by centrifugation, as described (Nguyen, et al., Biochemistry 49:10881-9 [2010]).

The partitioning of the lipidated ApoE4 between human HDL3 and VLDL is monitored using a previously described, competitive-binding assay. In brief, ¹⁴C-ApoE4 (5 μg) is incubated at 4° C. for 30 min with 0.45 mg VLDL protein and 0.9 mg HDL3 protein (these concentrations give approximately equal total VLDL and HDL3 particle surface areas available for ApoE4 binding in the presence or absence of antibodies of the disclosure, in a total volume of 1 ml of Tris buffer (pH 7.4). VLDL, HDL3 and unbound ApoE4 are then separated by sequential ultracentrifugation. In another example, VLDL/HDL distribution in the presence or absence of antibodies is evaluated in human plasma in vitro (Sakamoto, et al., Biochemistry 47:2968-77 [2008]). ApoE2 and ApoE3 (ApoE2/3) partition differently between VLDL and HDL than ApoE4 when added to human plasma. ApoE2/3 and ApoE4 bind similarly to VLDL when added to a mixture of the two lipoproteins whereas ApoE2/3 binds markedly better than ApoE4 to HDL3. The VLDL/HDL distribution of ApoE2/3 and ApoE4 in the presence or absence of function changing antibodies is examined after each protein is added separately to human plasma.

### Example 10: Isolation of VLDL and HDL

VLDL and HDL3 are isolated by sequential density ultracentrifugation from a pool of fresh-frozen human plasma (similar results are obtained when lipoproteins from fresh plasma are utilized). The various ApoE preparations are trace-labeled with either ³H or ¹⁴C by reductive methylation and incubated at 4° C. for 30 min with a mixture of human HDL3 and VLDL. Each of the pair of ³H- and ¹⁴C-labeled proteins (5 μg) to be compared is mixed and incubated with 0.45 mg VLDL protein and 0.9 mg HDL3 protein (these concentrations give approximately equal total VLDL and HDL3 particle surface areas available for ApoE binding) in a total volume of 1 ml of Tris buffer (10 mM Tris-HCl, 150 mM NaCl, 0.02% NaN3, 1 mM EDTA, pH 7.4). The VLDL, HDL3 and unbound protein are separated by sequential density gradient ultracentrifugation and the amounts and ratios of ³H/¹⁴C radioactivity in each fraction are determined by liquid scintillation counting. Similar results can be obtained when the lipoproteins are isolated by gel filtration chromatography. Binding of ApoE isoforms or fragments thereof to VLDL and HDL particles in the presence or absence of antibodies of the present disclosure, using SPR analysis, can be used to determine affinity as well as kinetics (Sakamoto, et al., Biochemistry 47:2968-77 [2008].

HDL3 and VLDL are purified by sequential density ultracentrifugation from a pool of fresh human plasma obtained by combining several single units from normolipidemic individuals. Full-length human ApoE2/3, ApoE4, and their 22 kDa (residues 1-191), 12 kDa (residues 192-299), and 10 kDa (residues 222-299) fragments are expressed and purified. The C-terminal truncation variants (Δ251-299, Δ261-299, and Δ273-299) of ApoE2/3 and ApoE4 are created as described previously. The ApoE preparations are at least 95% pure as assessed by SDS-PAGE. In all experiments, the ApoE sample is freshly dialyzed from a 6 M GdnHCl and 1% β-mercaptoethanol (or 5 mM DTT) solution into a buffer solution before use.

### Example 11: Biotinylation of HDL and VLDL Particles

HDL₃ and VLDL are dialyzed into phosphate-buffered saline (pH 7.4) prior to biotinylation (Nguyen, et al., Biochemistry 48:3025-32 [2009]). The EZ-link sulfo-NHS-LC-biotinylation kit from Pierce Chemical Co. (Rockford, Ill.) is used for attaching biotin molecules through a 2.24 nm spacer arm to lysine residues on the surface of the lipoprotein particles. HDL3 and VLDL, each at 1.0 mg of protein/mL, are mixed with a freshly made 10 mM sulfo-NHS-LC-biotin solution at a 10-fold molar excess of biotin. The lipoproteins are incubated under nitrogen at 4° C. overnight before dialysis against Tris-buffered saline (TBS, pH 7.4) to remove unreacted sulfo-NHS-LC-biotin. The degree of biotinylation of the particles is determined using conditions recommended by Pierce. Briefly, solutions containing biotinylated lipoproteins are added to a mixture of HABA reagent (2-(4′-hydroxyphenyl)azobenzoic acid) and immunopure avidin (Pierce Chemical Co.). Because of its higher affinity for avidin, biotin, from the biotinylated lipoproteins, displaces avidin-bound HABA. Therefore, the absorbance at 500 nm of the HABA-avidin complex is reduced. The change in absorbance is used to calculate the level of biotin incorporated into the lipoprotein particles. This procedure yields an average degree of labeling of one biotin molecule per lipoprotein particle.

### Example 12: Surface Plasmon Resonance (SPR) Determination of ApoE4 Binding to VLDL and HDL

Studies of the binding of apolipoproteins (association and dissociation) to HDL3 and VLDL are performed with a Biacore 3000 SPR instrument (Biacore, Uppsala, Sweden) using SA sensor chips (Biacore) (Nguyen, et al., Biochemistry 48:3025-32 [2009]). This chip is designed to bind biotinylated ligands through a high-affinity capture process. Prior to immobilization of HDL3 or VLDL on the sensor chip, the streptavidin surface is conditioned with three consecutive 1 min injections of 1 M NaCl in 50 mM NaOH (50 μL/min). The biotinylated HDL3 or VLDL is then immobilized onto the surface through the quasi-covalent biotin-streptavidin interaction by exposing the surface to the biotinylated lipoprotein solutions in running buffer (50 mM TBS, pH 7.4) until 2500-3000 and 5000-7000 response units (RU) of biotinylated HDL3 or VLDL, respectively, are bound to the surface. This is achieved by a 10 μL injection of biotinylated HDL3 or VLDL (1.0 mg of protein/mL) at a flow rate of 2 μL/min, at room temperature. After 5 min, the chip is washed with degassed TBS to remove unattached lipoprotein. A 50 μg/mL human apoE3 solution is passed over the chip at a rate of 20 μL/min for 2 min to block any remaining hydrophobic surface areas and reduce the subsequent level of ApoE binding to nonlipoprotein sites. The chip is then washed with TBS until the SPR signal reached a steady background value.

The surface of the immobilized HDL3 or VLDL is then exposed to a 4 min injection of ApoE dissolved in degassed TBS at a flow rate of 20 μL/min to monitor association, and then TBS alone is passed over the sensor surface to monitor dissociation of ApoE from the immobilized lipoprotein particles (Nguyen, et al., Biochemistry 48:3025-32 [2009]). For these experiments, two flow cells are monitored simultaneously with flow cells 1 and 2 containing immobilized biotinylated VLDL and HDL3, respectively. A sensor chip lacking immobilized lipoprotein can not be used as a reference cell because ApoE binds more to this surface than to a lipoprotein-coated chip. The apolipoproteins are dialyzed from 6 M GdnHCl containing 5 mM DTT into TBS, filtered (Ultrafree-MC centrifugal filter devices, 0.1 μm filter unit, Millipore, Bedford, Mass.), and degassed before serial dilutions (2.5-50 μg/mL) are made just prior to injection. The sensor chip is washed two times with 20 μL of TBS between each injection of apolipoprotein. The chips are used for 2 days in repetitive experiments. Regeneration of the sensor chip surface is not possible since the lipoproteins are directly immobilized via biotin-streptavidin interaction. The ApoE sensorgrams are independent of flow rate in the range of 10-40 μL/min, indicating that ApoE binding at 20 μL/min is not limited by mass transport (diffusion) effects.

Steady-state binding isotherms and K_{d} values of the binding to HDL3 and VLDL are obtained by generating sensorgrams at different apoE concentrations. The sensorgrams are analyzed with the BIA evaluation software, version 4.1 (Biacore). The response curves of various apolipoprotein (analyte) concentrations are fitted to the two state binding model described by the following equation:

$$A + B {{}^{{k}_{al}}}_{{k}_{dl}} AB {{}_{{k}_{d 2}}}^{{k}_{a 2}} {AB}_{X}$$

The equilibrium constants of each binding step are K₁=kₐ₁/k_{d1} and K₂=kₐ₂/k_{d2}, and the overall equilibrium binding constant is calculated as Kₐ=K₁(1+K₂) and K_{d}=1/Kₐ. In this model, the analyte (A) binds to the ligand (HDL3 or VLDL) (B) to form an initial complex (AB) and then undergoes subsequent binding or conformational change to form a more stable complex (ABₓ). A further check of the two-state binding mechanism is obtained by variation of the contact time for association between apoE and the lipoprotein particle. For a two-state reaction, an increase in the contact time between the analyte and the ligand decreases the dissociation rate since more of the stable ABₓ complex is formed. For the apolipoproteins, binding responses in the steady-state region of the sensorgrams (R_{eg}) are also plotted against apolipoprotein concentration (C) to determine the overall equilibrium binding affinity. The data are subjected to nonlinear regression fitting (Prism 4, GraphPad Inc.) according to the following equation:

R_{eg}=CRₘₐₓ/(C+K_{d})

Rₘₐₓ is the maximum binding response, and K_{d} is the dissociation constant. This SPR approach for measuring K_{d} is validated by the fact that monitoring the binding of ApoE3 and ApoE4 to VLDL by ultracentrifugation yields similar K_{d} values.

### Example 13: ApoE4 Interaction with Lipid Measured Using a DMPC Clearance Assay

To assess the lipid-binding abilities of ApoE, a DMPC clearance assay is used (Nguyen, et al., Biochemistry 48:3025-32 [2009]). ApoE2/3 and ApoE4 at a concentration of 0.1 mg/ml display a time-dependent decrease in light scattering intensity with ApoE4 giving a faster rate than ApoE2/3. Such faster clearance rates for ApoE4 than ApoE3 are seen over a wide range of ApoE concentrations, indicating that ApoE4 has a stronger ability to solubilize DMPC vesicles than ApoE3. This stronger ability of ApoE4 to solubilize DMPC vesicles is reduced by introduction of the mutation E255A. Removal of residues 273-299 in both ApoE3 and ApoE4 enhances the clearance activities to a similar level for the isolated C-terminal fragments, whereas further truncated mutants 1-260 and 1-250 display greatly reduced clearance activities. Antibodies that change the clearance rates of lipidated ApoE4 to exhibit greater similarity to (e.g., mimic) the clearance rate of ApoE2/3 are identified.

### Example 14: Measuring Lipidated ApoE4 Dependent Blood-Brain Barrier Leakage In Vivo

ApoE4 leads to increased blood-brain permeability (BBB), compared to ApoE2 and/or ApoE3. Antibodies of the present disclosure, which decrease the BBB permeabity induced by lipidated ApoE4, such that the ApoE4 exhibits (e.g., mimics) effects on permeability with greater similarity (e.g., more similar) to that observed in vivo, in an animal, or in cell culture models of the BBB for ApoE2 and/or ApoE3 are identified in one or more assays, such as for example, those described below (Nishitsuji, et al., J Biol Chem 286:17536-42 [2011]). More specifically, in one in vivo example, mice expressing human ApoE are generated by the gene-targeting technique taking advantage of homologous recombination in embryonic stem cells (knock-in)(Nishitsuji, et al., J Biol Chem 286:17536-42 [2011]). Three week-old C57BL/6 mice are purchased from SLC Inc. (Hamamatsu, Japan). For astrocyte culture, pregnant C57BL/6 mice are purchased from SLC Inc., and newborn mice at postnatal day 2 are used for the experiment. ApoE-KO mice are obtained from the Jackson Laboratories (Bar Harbor, Me.).

BBB permeability is quantified using the established Evans blue dye assay technique assay (Nishitsuji, et al., J Biol Chem 286:17536-42 [2011]). Two hundred microliters of 20% mannitol (Sigma) is injected into 6-month-old aApoE knock-in mice through the tail vein. After 30 min, 200 microliters of 2% Evans blue (Sigma) was injected intraperitoneally. Mice are sacrificed at 3 h after injection. The cerebellum and cerebral cortex are collected and then incubated in 500 ml of formamide for 72 h in the dark. Subsequently, the absorption (A) of the extracted dye is measured at 630 nm by spectrophotometry.

### Example 15: Measuring Lipidated ApoE4 Dependent Blood-Brain Barrier Leakage In Vitro

Primary cultures of mouse brain capillary endothelial cells (mBECs) are prepared from 3-week-old mice in accordance with previously described methods (Nishitsuji, et al., J Biol Chem 286:17536-42 [2011]). The mice are sacrificed, and the gray matter is dissected out. The gray matter is minced in ice-cold Dulbecco's modified Eagle's medium (DMEM) (Invitrogen) and then dissociated into single cells by 25 times of up- and down-strokes with a 5-ml pipette in 10 ml of DMEM containing 100 ul of collagenase type 2 (100 mg/ml; Sigma), 150 ul of DNase I (1 mg/ml; Roche Applied Science), followed by digestion for 1.5 h at 37° C. The digest in 20% bovine serum albumin (BSA) (Sigma) in DMEM is centrifuged at 1,000×g for 20 min to obtain cell pellets. The microvessels obtained from the pellets are further digested with collagenase and dispase (1 mg/ml; Roche Applied Science) for 1 h at 37° C. Microvessel endothelial cell clusters are separated on a 33% continuous Percoll (Pharmacia) gradient, collected, and washed twice in DMEM before plating on 60-mm plastic dishes coated with collagen type IV (Nitta Gelatin) and fibronectin (Calbiochem) (both 0.1 mg/ml). mBEC cultures are maintained at 37° C. for 2 days in DMEM/F12 (Invitrogen) supplemented with mBEC medium I containing 10% FBS, basic fibroblast growth factor (1.5 ng/ml; Roche Applied Science), heparin (100 ug/ml; Sigma), insulin (5 ug/ml; Sigma), transferrin (5 ug/ml; Sigma), sodium selenite (5 ng/ml; Sigma) (insulintransferrin-sodium selenite media supplement), penicillin, streptomycin (Invitrogen), and puromycin (4 ug/ml; Sigma).

On the 3rd day, the medium is replaced with a new medium that contains all of the components of mBEC medium I except puromycin (mBEC medium II). When the cultures reach 80% confluence (approximately 4th day in vitro), the purified endothelial cells are passaged and used. Pure cultures of mouse cerebral pericytes are obtained by a 2-week culture of isolated brain microvessel fragments, which contain pericytes beside endothelial cells. When the cultures reach confluence, cells are treated with trypsin (Invitrogen), replated onto uncoated dishes, and cultured in DMEM supplemented with 10% FBS. Culture medium is changed every 3 days. Highly astrocyte-rich cultures are prepared using previously described methods. In brief, brains of day 2 postnatal human ApoE-knock-in mice, WT mice, or ApoE-KO mice are removed under anesthesia. The cerebral cortices from the mice are dissected, freed from meninges, and diced into small pieces. The cortical fragments are incubated in 0.25% trypsin and 20 mg/ml DNase I in PBS at 37° C. for 20 min. The fragments are then dissociated into single cells by pipetting. The cells are seeded in 75-cm² dishes with DMEM-containing 10% FBS at a density of 5×107 cells/dish. After 10 days of incubation, flasks are shaken at 37° C. overnight, and the remaining astrocytes in the monolayer are trypsinized (0.1%) and reseeded. The astrocyte-rich cultures are maintained in DMEM-containing 10% FBS until use.

Barrier integrity in in vitro BBB models is analyzed by measurement of transendothelial electric resistance (TEER). TEER is measured using an epithelial-volt-ohm meter and Endohm-24 chamber electrodes (World Precision Instruments). TEER of coated but cell-free filters is subtracted from the measured TEERs of models. To construct in vitro models of BBB, pericytes (1.5×10⁴ cells/cm²) are seeded on the bottom side of the polyester membrane of Transwell inserts (Corning Inc., Corning, N.Y.) coated with collagen type IV and fibronectin. The cells are allowed to adhere firmly overnight, then endothelial cells (1.5×10⁵ cells/cm2) are seeded on the upper side of the inserts placed in the wells of 24-well culture plates (for measurement of TEER) or 6-well plates (for Western blotting). Astrocytes (1×10⁵ cells/cm2) on the 6-well plates or 24-well plates are maintained in mBEC medium II. Finally, the Transwell inserts with mBECs and pericytes are placed into the 6-well or 24-well plates with astrocytes and maintained for 7 days. For the experiment to examine the effect of lipidated ApoE-containing medium on BBB integrity, the double co-cultured model using pericytes and mBECs in the absence of astrocytes is used. For the preparation of conditioned media, primary astrocytes prepared from ApoE3- or ApoE4-knock-in mice are cultured in mBEC medium II for 48 h, and the conditioned media of ApoE3-expressing astrocytes (apoE3-CM) or ApoE4-expressing astrocytes (apoE4-CM) are collected. To determine the effect of ApoE3-CM or ApoE4-CM on BBB integrity, each CM is added only to the luminal side of the double co-cultured model, and the abluminal side is filled with mBEC medium II. These culture media are replaced with newly prepared CM or fresh mBEC medium II on the 3rd and 5th days and TEER is determined on the 7th day.

### Example 16: Assay for Lipidated ApoE4 Dependent Blood-Brain Barrier Permeability

Many assays of the integrity and health of the pericytes that make up the BBB are known in the art and can be utilized (Bell, et al., Nature 485:512-6 [2012]), including for example: (a) multiphoton microscopy of tetramethylrhodamineconjugated dextran (TMR-dextran) (b) Cyclophilin A (CYPA) levels, as this is a pro-inflammatory mediator in brain microvessels (c) Systemically administered cadaverine accumulation in brain (d) Endogenous IgG leakage, thrombin and fibrin accumulation in brain (e) Haemosidrin foci (in terms of Prussian Blue) (f) metalloproteinases (MMP)2 and MMP9 (gelatinases) accumulation by IHC or Western blot. (g) Gelatin zymography of brain tissue for pro-MMP9 and activated MMP9 and MMP2 levels (h) Levels of MMP9 substrates including collagen IV and tight-junction proteins ZO-1 (also known as Tjp1), occludin and claudin 5, which are required for normal BBB integrity in brain microvessels (i) Nuclear accumulation in pericytes of Nuclear-factor-kB (NF-kB), which transcriptionally activates MMP9 in cerebral vessels, causing BBB breakdown.

### Example 17: Processing of APP to Amyloid Beta In Vitro

In another example, ApoE4 antibodies are identified that suppress the higher levels (e.g., increased) of amyloid beta production seen in cells treated with ApoE4 (e.g., lipidated ApoE4) to levels more similar to the levels observed in cells treated with ApoE2 and/or ApoE3 (e.g., lipidated ApoE2, ApoE3) (He, et al., J Neurosci 27:4052-60 [2007]). Neuroblastoma N2a-APPsw cells are cultured in 24-well plates in DMEM containing 10% fetal bovine serum, 100 U/ml penicillin, and 100 μg/ml streptomycin (Invitrogen) with 80 μg/ml G418, one day before use. Transient transfections are done using FuGENE6 (Roche Diagnostics) and fresh Optimum medium (Invitrogen) is supplied 5 h after transfection. Apolipoproteins are added in the fresh medium at 10 μg/ml. Conditioned medium is collected 24 h later. Aβ40 or Aβ42 is determined in triplicate using an Aβ40 or Aβ42 ELISA Kit (Biosource International, Camarillo, Calif.).

In another example, ApoE4 antibodies that increase clearance of amyloid beta from brain tissue and interstitial fluid (ISF) (e.g., of ApoE4 carriers) to levels more similar to the levels observed in cells treated with ApoE2 and/or ApoE3 are identified (Castellano, et al., Sci Transl Med 3:89ra57 [2011]).

### Example 18: Processing of APP to Amyloid Beta In Vivo

Homozygous PDAPP (APPV717F) mice lacking ApoE on a mixed background composed of DBA/2J, C57BL/6J, and Swiss Webster are crossed with mice expressing ApoE2, ApoE3 and ApoE4 under control of mouse regulatory elements on a C57BL/6J background (Castellano, et al., Sci Transl Med 3:89ra57 [2011]). Resulting mice are intercrossed to generate homozygous PDAPP/TRE mice, which are then maintained via a vertical breeding strategy. Male and female PDAPP/TRE mice are used throughout experiments. For experiments involving TRE mice with murine APP, 2.5-month-old male littermates on a C57BL/6J background from each ApoE genotype are purchased from Taconic.

In vivo microdialysis is performed in the left hemisphere of 20- to 21-month-old mice, after which mice are immediately perfused transcardially, fixing brains in 4% paraformaldehyde overnight. After brains are placed in 30% sucrose, the contralateral (noncannulated) hemisphere is sectioned on a freezing-sliding microtome. Serial 50-μm coronal sections are taken from the rostral anterior commissure through the caudal extent of the hippocampus, staining sections with biotinylated 3D6 antibody (anti-Aβ₁₋₅) for amyloid β immunostaining quantification and X-34 dye for amyloid load quantification. Slides are scanned in batch mode with the NanoZoomer slide scanner system (Hamamatsu Photonics), capturing images in bright-field mode (amyloid β immunostaining) or fluorescent mode (X-34). NDP viewer software is used to export images from slides before quantitative analysis with ImageJ software (National Institutes of Health (NIH)). Using three sections per mouse separated each by 300 μm (corresponding to bregma −1.7, −2.0, and −2.3 mm in mouse brain atlas), the percentage of area occupied by immunoreactive amyloid β or amyloid (X-34-positive signal) is determined in a blinded fashion, thresholding each slide to minimize false-positive signal, as described.

In vivo microdialysis in 20- to 21-month-old and 3- to 4-month-old PDAPP/TRE mice is performed essentially as described to assess steady-state concentrations of various analytes in the hippocampal ISF with a 38-kD cutoff dialysis probe (Bioanalytical Systems Inc.). ISF exchangeable AP₁₋ₓ(eAβ₁₋ₓ) is collected with a flow rate of 1.0 μl/min, whereas ISF eAβₓ₋₄₂ and urea are collected with a flow rate of 0.3 μl/min. For clearance experiments, a stable baseline of ISF eAβ₁₋ₓ concentration is obtained with a constant flow rate of 1.0 μl/min before intraperitoneally injecting each mouse with 10 mg/kg of a selective y-secretase inhibitor (LY411,575), which is prepared by dissolving in dimethyl sulfoxide (DMSO)/PBS/propylene glycol. The elimination of eAβ₁₋ₓ from the ISF follows first-order kinetics; therefore, for each mouse, t_{1/2} for eAβ is calculated with the slope, k′, of the linear regression that includes all fractions until the concentration of eAβ stops decreasing. Microdialysis using the zero flow extrapolated method is performed by varying the flow rates from 0.3 to 1.6 μl/min. Zero flow data for each mouse are fit with an exponential decay regression with GraphPad Prism 5.0 software.

Quantitative measurements of amyloid β collected from in vivo microdialysis fractions are performed with sensitive sandwich ELISAs. For human Aβ₁₋ₓ quantification, ELISA plates are coated with m266 antibody (anti-Aβ₁₃₋₂₈), and biotinylated 3D6 antibody (anti-Aβ₁₋₅) is used for detection. For Aβₓ₋₄₂ ELISAs, HJ7.4 (anti-Aβ₃₅₋₄₂) antibody is used to capture, followed by biotinylated HJ5.1 antibody to detect (anti-APβ₁₃₋₂₄).

### Example 19: Lipidated ApoE4 Dependent b-Secretase Activity in Hippocampal Homogenates from Young PDAPP/TRE Mice

After transcardial perfusion with heparinized PBS, brain tissue is microdissected and immediately frozen at −80° C. Hippocampal tissue is manually Dounce-homogenized with 75 strokes in radioimmunoprecipitation assay (RIPA) buffer (50 mM tris-HCl (pH 7.4), 150 mM NaCl, 0.25% deoxycholic acid, 1% NP-40, 1 mM EDTA) containing a cocktail of protease inhibitors (Roche). Total protein concentration in hippocampal homogenates is determined with a BCA protein assay kit (Pierce). Equivalent amounts of protein (50 μg) are loaded on 4 to 12% bis-tris gels (Invitrogen) for SDS-PAGE before transferring protein to 0.2-μm nitrocellulose membranes. Immediately after transfer, blots are boiled for 10 min before blocking and incubation with 82E1 antibody (anti-Aβ₁₋₁₆; IBL) to detect C99 the transmembrane carboxyl-terminal domain of the amyloid precursor protein that is cleaved by γ-secretase to release the amyloid-β. Loading is normalized by stripping blots and reprobing with α-tubulin antibody (Sigma). Normalized band intensities are quantified with ImageJ software (NIH).

β-Secretase activity in hippocampal lysates is assessed with a commercially available kit (#P2985; Invitrogen) that relies on fluorescence resonance energy transfer (FRET) that results from (3-secretase cleavage of a fluorescent peptide based on the APP sequence (Rhodamine-EVNLDAEFK-Quencher). Briefly, 5 μg of protein per sample is mixed with sample buffer and β-secretase substrate, monitoring fluorescence signal every minute for 120 min with a Synergy2 BioTek (BioTek Instruments Inc.) plate reader (excitation, 545 nm/emission, 585 nm). Because the kinetics of the reaction for all samples is reliably linear in the 20- to 60-min interval, reaction velocity (relative fluorescence units (RFUs) per minute) is calculated and reported over this interval for all samples. Specificity of β-secretase activity is validated with a commercially available β-secretase inhibitor.

### Example 20: Analyses of Lipidated ApoE4 Dependent Brain Amyloid β Clearance by Stable Isotopic Labeling Kinetics

Fractional synthesis rate FSRs of amyloid β are measured in hippocampal lysates from young PDAPP/TRE mice with a method adapted from the in vivo stable isotopic labeling kinetics technique. Briefly, after mice are injected intraperitoneally with (¹³C₆)leucine (200 mg/kg), brain tissue harvesting and plasma collection are performed 20 and 40 min after injection. Whole hippocampus is lysed with 1% Triton X-100 lysis buffer containing protease inhibitors and amyloid β in the extracts is immunoprecipitated with HJ5.2 antibody (anti-Aβ₁₃₋₂₈). After trypsin digestion of immunoprecipitated amyloid β, LC-MS is performed to measure the relative abundance of labeled to unlabeled tryptic amyloid β peptide, which is calibrated with a standard curve of amyloid β secreted from H4 APP695ΔNL neuroglioma cells. FSR curves are then generated based on the amount of labeled to unlabeled amyloid β present 20 and 40 min after (¹³C₆) leucine injection, normalized to the amount of free leucine in the plasma, which is measured by gas chromatography (GC)-MS (Deane, et al., J Clin Invest 118:4002-13 [2008]). Aβ40 and Aβ42 are obtained from the W.M. Keck Foundation Biotechnology Resource Laboratory (Yale University, New Haven Conn., USA). They are synthesized by solid-phase F-moc (9-fluorenylmethoxycarbonyl) amino acid chemistry, purified by reverse-phase HPLC, and structurally characterized. Lyophilized peptides are kept at −80° C. until used.

### Example 21: Lipidated ApoE4 Dependent Formation of Amyloid β-ApoE Complexes

Lipidated and lipid-poor (e.g., non-lipidated)¹²⁵I-labeled ApoE2 and ApoE4 complexes with synthetic human Aβ40 and Aβ42 are prepared with a ratio of amyloid β to ApoE of 40 to 1. Complexes ae purified by fast flow size-exclusion chromatography (FPLC) to remove excess free Aβ. Formation of complexes between lipidated ApoE and lipid-poor ApoE isoforms with amyloid β isoforms and complete removal of excess free amyloid β are verified by nondenaturing 4%-20% Tris-glycine polyacrylamide gel (Invitrogen) and 10%-20% Tris-tricine polyacrylamide gel (Bio-Rad), respectively, followed by Western blot analysis for ApoE. ¹²⁵I-labeled Aβ40 or Aβ42 complexes with unlabeled ApoE2 and ApoE4 also are prepared in the same way as described above (Deane, et al., J Clin Invest 118:4002-13 [2008].

### Example 22: Analyses of ApoE4 Dependent Brain Amyloid β Clearance Using Amyloid β Tracer

The amount of injected tracers is accurately determined using a micrometer to measure the linear displacement of the syringe plunger in the precalibrated microsyringe. Mock CSF (0.5 μl) containing ¹²⁵I-labeled test-tracers Aβ (monomer), ApoE (lipid poor or lipidated), or Aβ-apoE complex together with ¹⁴C-inulin (reference molecule) is microinfused into brain ISF over 5 minutes. When the effects of different unlabeled molecular reagents are tested, they are injected 15 minutes prior to radiolabeled ligands and then simultaneously with radiolabeled ligands, as described (Deane, et al., J Clin Invest 118:4002-13 [2008]).

At the end of the experiments, brain, blood, and CSF are sampled and prepared for radioactivity analysis and TCA and SDS-PAGE analyses to determine the molecular forms of test tracers. Studies with ¹²⁵I-labeled amyloid β have demonstrated that both radiolabeled Aβ40 and Aβ42 remain mainly intact in brain ISF (>95%) within 30-300 minutes of in vivo clearance studies as well as during short-term kinetic clearance studies in vitro on brain capillaries In the present study, we confirmed previous findings indicating that molecular forms of transport of ¹²⁵I-labeled Aβ and apolipoproteins within 30-300 minutes of clearance studies remained mainly in their original form of intact molecules, as injected in the CNS.

### Example 23: Analyses of Lipidated ApoE4 Dependent High Cholesterol and Diabetes

Mice homozygous for replacement of the endogenous ApoE gene with the human ApoE*3 (E3) or ApoE*4 (E4) allele are crossed with mice deficient in the LDLR (Johnson, et al., J Lipid Res 54:386-96 [2013, Johnson, et al., Diabetes 60:2285-94 [2011]). All mice are on C57BL/6 backgrounds. Male mice are fed normal chow diet ad libitum (5.3% fat and 0.02% cholesterol; Prolab IsoPro RMH 3000). Diabetes is induced at 2 months of age by peritoneal injections of STZ for 5 consecutive days (0.05 mg/g body wt in 0.05 mol/L citrate buffer, pH 4.5). Mice maintaining glucose levels >300 mg/dL throughout the course of the study are considered “diabetic.” “Non-diabetic” control mice are injected with vehicle citrate buffer. Biochemical analyses are carried out at 1 month post-STZ unless otherwise stated.

After a 4-h fast, animals are anesthetized with 2,2,2-tribromoethanol and blood is collected. Plasma glucose, cholesterol, phospholipids, free fatty acids (FFAs), and ketone bodies are measured using standard commercial kits (Wako, Richmond, Va.). TGs and insulin are determined using standard commercial kits from Stanbio (Boerne, Tex.) and Crystal Chem Inc. (Downers Grove, Ill.), respectively. Liver TGs are extracted. Lipoprotein distribution and composition is determined with pooled (n=6-8) plasma samples (100 μL) fractionated by fast-protein liquid chromatography using a Superose 6 HR10/30 column (GE Healthcare, Piscataway, N.J.). Pooled plasma (800 μL) is separated by sequential density ultracentrifugation into density fractions from <1.006 g/mL (VLDL) to >1.21 g/mL (HDL) and subjected to electrophoresis in a 4-20% denaturing SDS-polyacrylamide gel. Carboxylmethyl lysine (CML) advanced glycation end products (AGEs) are measured using an ELISA with antibodies specific for CML-AGEs (CycLex, Nagano, Japan).

ApoE and ApoCIII are measured using an ELISA with antibodies specific for ApoE (Calbiochem, San Diego, Calif.) and ApoCIII (Abcam, Cambridge, Mass.). Protein expression by Western blot is determined using antibodies against AMP-activated protein kinase (AMPK)-α, phosphorylated (Thr172) AMPK (pAMPK)-α, acetyl-CoA carboxylase (ACC), phosphorylated (Ser79) ACC (pACC), and β-actin (Cell Signaling, Boston, Mass.). Lipid tolerance test is performed by gavaging 10 mL/kg olive oil after an overnight fast. For VLDL secretion, plasma TG is measured after injection of Tyloxapol (Triton WR-1339, Sigma, St. Louis, Mo.) via tail vein (0.7 mg/g body wt) after a 4-h fast. VLDL lipolysis is estimated by incubating VLDL (25 μg TG in 60 μL PBS) at 37° C. with 15 units of bovine lipoprotein lipase (Sigma). The reaction is stopped by adding 3 μL of 5 mol/L NaCl, and fatty acid release (FAₜᵢₘₑₚₒᵢₙₜ−FA₀) is measured as above.

### Example 24: Analyses of Lipidated ApoE4 Dependent Atherosclerosis

After 3 months of diabetes, mice are killed with a lethal dose of 2,2,2-tribromoethanol and perfused at physiological pressure with 4% phosphate-buffered paraformaldehyde (pH 7.4). Morphometric analysis of plaque size at the aortic root is performed as described. Apoptotic cells are detected in 8-μm frozen sections of the aortic root with a kit that detects DNA fragmentation (Chemicon, Billerica, Mass.). Macrophages are detected with a 1:500 dilution of MOMA-2 (Abcam) and a 1:2,000 dilution of goat polyclonal secondary antibody to rat IgG—H&L Cy5 (Abcam) (Johnson, et al., J Lipid Res 54:386-96 (2013, Johnson, et al., Diabetes 60:2285-94 [2011]).

### Example 25: Analyses of Lipidated ApoE4 Dependent Pathological Inflammation and Pathological Microglial Activity

ApoE4 mice display increased acute pathological inflammatory response, including cytokines expression response with peripheral LPS injection, as quantified by cytokine release both peripherally and in brain. ApoE4 antibodies that reduce acute pathological inflammatory response (e.g., in ApoE4 carriers) to levels more similar to these observed in animal expressing ApoE3 or ApoE3 are identified (Lynch, et al., J Biol Chem 278:48529-33 [2003]).

Cytokine levels in murine serum and brain homogenate are determined by using mouse cytokine ELISA kits for murine IL-6 and TNFa following the manufacturer's specifications (Pierce). Murine brains are isolated and quick-frozen by immersing in liquid nitrogen. The frozen brains are ground up into a fine powder in a liquid nitrogen pre-cooled mortar. Homogenates are generated by placing brains in ice-cold homogenized buffer (0.25 M sucrose, 1 mM EDTA, 10 mM HEPES, pH 7.4, 0.1% ethanol, and mixture tablets (Roche Applied Science)) and homogenized by using a Teflon pestle and a motor-driven tissue homogenizer. Samples are maintained on ice throughout the homogenization procedure. After homogenization, the sample is clarified by centrifuging at 5° C. for 15 min at 1,500×g to remove cellular debris. The supernatant is removed and divided into several smaller working aliquots and stored at −70° C. until ELISA analysis.

### Example 26: Lipidated ApoE4 Dependent Pathological Astroglial and Microglial Activation In Vivo

ApoE4 antibodies are identified that reduce acute pathological inflammatory response to LPS (e.g., in ApoE4 carriers) to levels more similar to the levels observed in cells treated with ApoE2 or ApoE3 (Zhu, et al., Glia 60:559-69 [2012])Homozygous human ApoE2, ApoE3, and ApoE4 knock-in (targeted-replacement) mice are used (Zhu, et al., Glia 60:559-69 [2012, Sullivan, et al., J Biol Chem 272:17972-80 [1997]). In these mice, exons 2-4 of the human ApoE2, ApoE3, and ApoE4 genes replace the corresponding genomic DNA at the mouse ApoE locus. These three mice colonies as well as ApoE knock-out mice are maintained at Taconic (Hudson, N.Y.). Experiments are performed on age-matched male animals at 4 months of age.

Mice are anesthetized by intraperitoneal injection of 120 mg/kg ketamine (Abbott Laboratories, Chicago, Ill.) and then receive unilateral ICV injection of LPS (Sigma, St. Louis, Mo.) or vehicle control. Mice are injected with 2.5 μL of 400 ng/μL LPS or 2.5 μL of saline at a rate of 0.5 μL/min, using a syringe pump at the following mouse brain coordinates: anterior/posterior=−0.34 mm, medial/lateral=1.0 mm, dorsal/ventral=−2.0 mm (n=4-5 per treatment group). After each injection, the syringe is left for an additional 2 min to avoid liquid reflux. These mice are anesthetized with 120 mg/kg ketamine and euthanized by transcardial perfusion with ice-cold phosphate-buffered saline (1×PBS) containing 1× protease inhibitor cocktail (Calbiochem, Gibbstown, N.J.). For immunohistochemistry, the ipisilateral hemisphere is fixed in 4% paraformaldehyde in 1×PBS, pH7.4, for 48 h and then stored in 30% sucrose, 1×PBS solution for 24 h at 4° C. The contralateral hemisphere is immediately dissected on ice to obtain cerebral cortex, hippocampus, and cerebellum that are snap-frozen in liquid nitrogen and stored at −80° C. for biochemical analyses. Experiments are conducted on brains 24 or 72 h after ICV injection of LPS or vehicle control. These times are chosen to represent early and late responses to inflammation (Zhu, et al., Glia 60:559-69 [2012]).

The ipsilateral hemispheres are subsequently cut into 35 μm coronal sections on a Leica SM 2000R microtome, and sections are stored at −20° C. in 24-well plates with cryoprotectant (30% glycerol, 30% ethylene glycol, 1×PBS). Every sixth section is immunohistochemically processed for identification of glial cells using a rabbit antibody against Glial Fibrillary Acidic Protein (GFAP) (1:500, Dako, Carpinteria, Calif.) for astrocytes, and rat anti-F4/80 monoclonal antibody (1:500, Serotec, Raleigh, N.C.) for microglia/macrophage. Sections are incubated with the primary antibodies at room temperature overnight, washed with TBS-T (25 mM Tris-HCl, 137 mM NaCl, 2.7 mM KCl, pH 7.4, 0.25% Triton X-100), and then incubated at room temperature for 1 h with the corresponding biotinylated goat anti-rabbit and goat anti-rat IgG secondary antibodies. Sections are then incubated in peroxidase-conjugated avidin-biotin complex for 1.5 h. A chromogen solution containing 0.05% 3, 3′-diaminobenzidine and 0.003% H₂O₂ is used to obtain brown staining. The total numbers of F4/80-immunoreactive (F4/80-IR) microglia and GFAP-IR astrocytes in the hippocampus are determined using the computerized optical dissector method with Stereo Investigator software (Version 9.03, MBF Bioscience, Williston, Vt.) with Zeiss Imager Al microscope.

Cells are manually designated by a blinded investigator. The total numbers (N) of IR cells are calculated using the formula N=NV×V, where NV is the numerical density and V is the volume of the hippocampus or frontal cortex. The densities of the F4/80-IR and GFAP-IR cells in the hippocampus ipsilateral to the LPS injection site are determined in three sections per animal, and the average of the counts thus obtained is taken from four to five animals in each group.

Expression of the T-cell marker CD3 (1:250, Abcam, Cambridge Mass.), presynaptic marker synaptophysin (1:1,000, Chemicon), and postsynaptic markers PSD-95 (1:500, Abcam, Cambridge Mass.) and drebrin (1:2,000, Abcam, Cambridge Mass.), are evaluated by single immunofluorescence staining (Zhu, et al., Glia 60:559-69 [2012]). Brain sections (35 μm) are first blocked by incubation with TBS-T solution containing 5% bovine serum albumin (BSA) for 1 h at room temperature. For PSD-95 staining, the brain sections are pretreated with 100 mg/mL of pepsin (DAKO) at 37° C. in a water bath for 5 min prior to blocking. The sections are then incubated with primary antibodies dissolved in TBS solution containing 0.1% Triton X-100 and 2% BSA for 16 h at room temperature. The bound primary antibodies are visualized by incubating the sections for 1 h at room temperature with Alexa 488- or 594-conjugated donkey anti-rabbit IgG (1:1,000, Invitrogen). The sections are then mounted on slides, and fluorescence images are captured using a confocal scanning laser microscope (LSM 510; Zeiss, Oberkochen, Germany) with a 40× or 63× oil-immersion lens. Images of CD3, synaptophysin, PSD-95 and drebrin are taken in the CA3 region of the hippocampus or the Layers 3-4 of frontal cortex. The numbers of CD3 IR-positive cells in the CA3 region are evaluated by Image J and expressed as numbers per mm².

The cerebral cortex, hippocampus, and cerebellum are homogenized with a polytron homogenizer (Brinkmann Instruments, Rexdale, Ontario, Canada) using 12 rapid pulses in ten volume of ice-cold lysis buffer (150-350 μL, 50 mM Tris-HCl, 150 mM NaCl, pH7.4, 1% Triton X-100, 1× protease inhibitor cocktail). Homogenates are centrifuged at 14,000 g for 30 min at 4° C. and the supernatants are collected for biochemical analyses. Total protein concentration is determined by BCA protein assay kit (Pierce, Rockford, Ill.).

Pro-inflammatory cytokines (IL-113, IL-6, and TNF-α) in the brain homogenates are determined using commercial cytokine ELISA kits following the manufacturer's instructions (R&D, Minneapolis, Minn.). Briefly, cytokine standards, samples, diluent buffers, and biotinylated anti-IL-10, IL-6, or TNF-α solutions are pipetted into each well. After 2-h incubation at room temperature, standards and samples are washed and incubated in streptavidin-HRP working solution for 1 h at room temperature. Absorbance is measured at 450 nm using a Molecular Devices microplate reader (Molecular Devices, Sunnydale, Calif.). The concentration of the IL-1β, IL-6, and TNF-α is determined against a seven-point standard curve. The quantity of IL-1β, IL-6, and TNF-α is expressed as pg/mg total protein.

For each hippocampal homogenate, 30-50 μg of total protein is separated by 4-12% Bis-Tris gel (Invitrogen). Separated proteins are transferred onto PVDF membranes and analyzed by Western blotting. The following primary antibodies from Abcam are used: rabbit anti-PSD-95 (1:3,000), rabbit anti-synaptophysin (1:2,000), mouse anti-a-tubulin (1:8,000), and mouse anti-drebrin antibody (1:1,000), respectively. After incubation with the appropriate HRP-conjugated secondary antibody, membranes are developed using ECL-enhanced chemiluminescence (Amersham, Piscataway, N.J.). The X-ray film is scanned and the density of bands is quantified using Image J software. The amount of protein is expressed as a relative value to the levels of a-tubulin.

### Example 27: Analysis of Lipidated ApoE4 Dependent Leukocyte Telomere Length

Qualified telomere length is a measure of aging. Telomere length can be measured, for example, in peripheral lymphocytes in human subjects and animal models. ApoE4 has been associated with accelerated aging as measured by aged dependent reduction in length of telomeres in ApoE4 carriers (Takata, et al., J Gerontol A Biol Sci Med Sci 67:330-5 [2012]). ApoE4 antibodies that prevent or reduce accelerated aging to levels more similar to these observed in cells treated with APOE2/3 as measured by aged dependent length of telomeres in APOE4 carriers are identified.

Subjects undergo blood sampling using venipuncture in a fasting state during the morning hours between 7 am to 10 am. All samples are processed for isolation of mononuclear cells within 1 h of collection. One ml of cryopreserved peripheral blood mononuclear cells (PBMCs) is thawed at 37° C., washed twice with 10 ml of cold DPBS (Invitrogen, Calsbard, Calif.). Cell pellets are collected and DNA is prepared using a Puregene DNA purification Kit (QIAGEN, Valencia, Calif.). Quantitative polymerase chain reaction (Q-PCR) is used to measure TL in the genomic DNA of peripheral leukocytes by determining the ratio of telomere repeat sequence copy number to a reference single copy gene copy number (T/S ratio) in each sample relative to a reference sample. The T and S values are each determined by the standard curve method using a serially diluted reference DNA and the T/S ratio is derived from the T and S value for each sample. Each T/S value is later converted to number of base pairs (bp). The conversion from T/S ratio to base pairs is calculated based on comparison of telomeric restriction fragment (TRF) length from Southern blot analysis and T/S ratios using DNA samples from the human cell line IMR90 at different population doublings. The slope of the linear regression line through a plot of T/S ratio (the x axis) versus mean TRF length (the y axis) is the number of base pairs of telomeric DNA corresponding to a single T/S unit. The formula to convert T/S ratio to base pairs is base pairs=3,274+2,413*(T/S). Results obtained with the Q-PCR method are strongly associated with the traditional terminal restriction fragment length index of TL obtained by Southern blot technique.

### Example 28: Effect of Anti-ApoE4 Antibodies on Recovery Form Traumatic Brain Injury

Antibodies of the present disclosure are evaluated for their ability to improve recovery from traumatic brain injury (TBI). ApoE4 carriers generally exhibit poorer recovery from traumatic brain injury compared to ApoE2/3 carriers. The ability of antibodies to improve recovery (e.g., inhibit or reduce) cognitive decline associated with traumatic brain injury, perinatal hypoxic-ischemic insult, stroke and/or epilepsy in ApoE4 carriers is quantified using any of the various animal models that are well described in the field. More specifically, for example, animals transgenic for ApoE4, other ApoE isoforms or control are treated with an insult, and then assayed for behavioral endpoints, pathological and biochemical changes, such as lesion volume, and microglial activation (Mannix, et al., J Cereb Blood Flow Metab 31:351-61 [2011]).

Mice are given free access to food and water and are housed in laminar flow racks in a temperature-controlled room with 12-hour day/night cycles. Transgenic mice that express targeted replacement of the mouse ApoE allele with human ApoE4 under the direction of the human glial fibrillary acidic protein promoter are obtained from Jackson Laboratories (Bar Harbor, Me., USA). Homozygous ApoE4 transgenic mice do not express endogenous mouse ApoE, develop normally, are fertile, are grossly phenotypically normal, and are congenic with C57Bl/6 (at least six backcrosses, Jackson Laboratories, 21 Jan. 2010). The murine ApoE primary sequence is the same as that of human ApoE4 in the polymorphic region (Arg 112), but is believed to behave like human ApoE3, because it lacks the Arg-61 domain interactions that confer the functional properties of ApoE4. Heterozygous ApoE4 mice have one copy of the wild-type (WT) murine ApoE allele. Male and female adult (aged 2 to 4 months) and immature (aged 20 to 21 days) ApoE4 mice are used in the two experimental protocols described below. Wild-type age- and gender-matched C57Bl/6 mice are used as controls. In all experiments, male and female ApoE4 and WT mice are distributed equally between groups.

The mouse CCI model is used as described previously (Mannix, et al., J Cereb Blood Flow Metab 31:351-61 [2011]) because this model reproduces cell death and cognitive deficits experienced by children and adults with severe TBI. Mice are anesthetized with 3% isoflurane, N₂O, and O₂ (2:1) and placed in a stereotactic frame. A 5-mm craniotomy is performed over the left parietotemporal cortex and the bone flap is removed. Controlled cortical impact is then produced using a pneumatic cylinder with a 3-mm flat-tip impounder, velocity 6 m/s, and impact depth of 0.6 mm. The scalp is sutured closed and mice are allowed to recover from anesthesia in their cages.

Gross vestibulomotor function is assessed using a wire grip test. The test consists of placing the mouse on a wire suspended between two poles and grading the degree of attachment and movement of the mouse. Scores are as follows: 0 is given to a mouse that fell from the wire within 30 seconds; 1 point for unilateral grasp of either upper or lower extremities, 2 points for midline grasp of both upper and lower extremities but not the tail; 3 points for midline grasp of all extremities plus the tail; 4 points for movement along the wire after achieving a score of 3; and 5 points for climbing down the pole within 60 seconds.

Investigators blinded to the mouse genotype evaluate the spatial memory performance of mice using the Morris water maze (MWM) task, as described (Mannix, et al., J Cereb Blood Flow Metab 31:351-61 [2011]). A white pool (83 cm diameter, 60 cm deep) is filled with water to 29 cm depth. Several highly visible intramaze and extramaze cues that remain constant throughout the trials are located in and around the pool. Water temperature is maintained at ˜24° C. The goal platform (a round, clear, plastic platform 10 cm in diameter) is positioned 1 cm below the surface of water. Each mouse is subjected to a maximum of two series of four trials per day. For each trial, mice are randomized to one of the four starting locations (namely north, south, east, or west) and placed in the pool facing the wall. Mice are given a maximum of 60 or 90 seconds to find and rest upon the submerged platform. If the mouse fails to reach the platform by the allotted time, it is placed on the platform by the investigator and allowed to remain there for 10 seconds. Mice are warmed and dried with a lamp between trials. For probe trials, mice are placed in the pool with the platform removed and the time that the animal swims in the target quadrant is recorded (maximum 60 seconds). For visible platform trials, the goal platform is marked by red tape and placed 0.5 cm above the water level. Performance in the MWM is quantitated by latency to the platform or latency in the target quadrant (probe trials).

Morphometric image analysis is used to determine the lesion size after CCI (Mannix, et al., J Cereb Blood Flow Metab 31:351-61 [2011]). Mice are anesthetized with isoflurane and killed by decapitation and the brains removed. Coronal sections (12 μm) are cut at 0.5 mm distances from the anterior to the posterior brain and mounted on poly-1-lysine-coated slides. The area of both hemispheres is determined using image analysis (Nikon Eclipse Ti 2000, MS Elements, MVI, Avon, Mass., USA). Lesion volume is obtained by subtracting the volume of brain tissue remaining in the left (injured) hemisphere from that of the right (uninjured) hemisphere, and expressed in mm³.

The brains are divided into left and right hemispheres. A small amount of the brain tissue anterior and posterior to the contusion is cut in the coronal plane and discarded. After recording the wet weight of the remaining brain tissue in each hemisphere, the brains are dried in an oven at 90° C. for 48 hours and dry brain weight is obtained. Percentage brain water content of each hemisphere is calculated as (wet-dry/wet) weight×100%. Brain edema is estimated as the difference in the percentage brain water content (injured-uninjured hemisphere).

Detergent soluble Aβ40 levels in the brain tissue are assessed because soluble but not insoluble (fibrillar) Aβ levels correlate with the extent of synaptic loss and severity of cognitive impairment in AD, and Aβ40 levels are normally higher in the brain compared with Aβ42. Cortical and hippocampal brain tissues in naive animals, pericontusional tissue including the cortex and the underlying hippocampus in acutely injured animals (48 hours), or cortical and hippocampal brain tissues surrounding the cavitary lesion from animals in chronic periods after CCI are used for determination of Aβ40. At various times after CCI, the brains are removed and bisected into injured and uninjured hemispheres. The brains are frozen in liquid nitrogen and stored at −80° C. until processing. Tissues samples are rapidly homogenized in 250 μL of RIPA buffer with protease inhibitor tablet (Sigma-Aldrich, St Louis, Mo., USA). After centrifugation (14,000 r.p.m., 4° C. for 15 minutes), the supernatant is collected (fraction 1), and the pellet is dissolved in 250 μL RIPA buffer and centrifuged again to obtain the supernatant (fraction 2). The two fractions are combined and protein content determined using the Bio-Rad (Hercules, Calif., USA) assay. Soluble Aβ40 is measured by sandwich enzyme-linked immunosorbent assay (Wako, Richmond, Va., USA) according to the manufacturer's instructions using sample protein concentrations of 1 to 2.5 mg/mL.

At 48 hours after CCI, mice are anesthetized and transcardially perfused with 4% paraformaldehyde 6 to 72 hours after injury. The brain is postfixed for 24 hours in 4% paraformaldehyde and cryoprotected in 30% sucrose for 24 hours. Coronal sections are cut (20 mm) and mounted on poly-1-lysine-coated slides. Sections are washed in phosphate-buffered saline, blocked in 3% normal goat serum in phosphate-buffered saline for 1 hour, and incubated overnight at 4° C. with rabbit anti-Iba-1 antibody (1:200; Wako Pure Chemical Industries, Osaka, Japan). Slides are washed in phosphate-buffered saline and incubated with the appropriate Cy3-conjugated secondary antibody (1:300; Jackson ImmunoResearch, West Grove, Pa., USA) for 60 minutes, washed in phosphate-buffered saline, and coverslipped. Brain sections are photographed on a Nikon Eclipse T300 fluorescence microscope (Nikon, Tokyo, Japan), using excitation/emission filters of 568/585 nm. For comparisons between groups, ×400 fields from the inferolateral aspect of the cortex underlying the contusion are randomly selected from brain regions at the level of the anterior hippocampus and photographed with identical camera settings by an observer blinded to the genotype, compared, and representative fields shown for qualitative analysis.

### Example 29: Effect of Anti-ApoE4 Antibodies on Recovery from Stroke

Antibodies of the present disclosure are evaluated for their ability to improve recovery from stroke. Ten-week-old weight-matched male 2/2-, 3/3-, or 4/4-KI mice are used in the study. In 4/4-KI mice, a portion of ApoE has been replaced by a transgene consisting of human ApoE4 cDNA through homologous recombination in embryonic stem cells, such that human ApoE proteins are expressed under the endogenous regulatory ApoE promoter region. Both 2/2 and 3/3-KI mice are produced using the same strategy as above, except that the transgenes carry ApoE2 or ApoE3 cDNA in place of ApoE4 cDNA. All of the KI mice used in the study are fully backcrossed onto the C57BL/6N background. The nucleotide sequences of the transgene apoE cDNAs are confirmed by sequencing cDNAs prepared from liver polyA+ RNAs of the three homozygous strains. Homozygosity is confirmed in each line of KI mice using allele-specific oligonucleotide primers and polymerase chain reaction analysis. The KI mice entirely lack mouse ApoE. Expression levels of human ApoE in neurons and in astrocytes, as well as the architecture of cerebral arteries including the presence or the absence of the posterior communicating artery, are comparable among the three lines of KI mice (Mori, et al., J Cereb Blood Flow Metab 25:748-762 [2005]).

All efforts are made to minimize animal suffering and to reduce the number of animals used. Animals are housed in a virus-free barrier facility under a 12/12 h light-dark cycle, with ad libitum access to food and water. All of the KI mice are subjected to fasting overnight (12 h) with free access to water before surgical procedures. Anesthesia is induced and maintained with halothane (1.5% to 2% and 0.5%, respectively) in a mixture of 70% nitrous oxide and 30% oxygen with spontaneous ventilation. As repeated blood withdrawal is likely to affect the outcome of pMCAO, all parameters (PaO2, PaCO2, pH, MABP, and blood glucose) are examined in separate sets of the arundic acid and the vehicle groups for 2/2-, 3/3-, or 4/4-KI mice (n=6 per each line of KI mice for each group, total n=36) under halothane anesthesia with spontaneous ventilation as described above. The femoral artery is cannulated to monitor MABP and to collect a blood sample. The above-mentioned physiologic parameters are recorded at 15 mins before (except for PaO2, PaCO2, and pH) and 30 mins after pMCAO. Rectal temperature is monitored throughout the operative procedure using a rectal probe, and normothermia is maintained with a homeothermic blanket control unit preset to 37° C. The distal segment of the middle cerebral artery (MCA) crossing over the rhinal fissure is exposed for induction of pMCAO. Briefly, a 1-cm skin incision is made approximately midway between the left outer canthus and anterior pinna. The temporalis muscle is incised and retracted to expose the squamous portion of the temporal bone. Under a surgical microscope, a burr hole (2 mm in diameter) is made by an electrical drill over the junction of the zygomatic process and the temporal bone. The dura mater is opened with a fine curved needle to expose the MCA. The left common carotid artery (CCA) is ligated with 8-0 silk, and then a 2-mm segment of the MCA is electrocauterized (Mori, et al., J Cereb Blood Flow Metab 25:748-762 [2005]).

The coagulated MCA segment is then transected with microscissors. Thereafter, the burr hole is covered with the temporalis muscle. After the skin is approximated, the wound is infiltrated with lidocaine. After halothane is discontinued, mice are returned to their cages and allowed free access to food and water. Evaluation of neurologic deficits is performed at 24-h intervals after pMCAO until euthanasia as follows: score 0, no neurologic deficit; score 1, forelimb flexion; score 2, decreased resistance to lateral push and forelimb flexion without circling; score 3, same behavior as grade 2, with circling, and score 4, inability to walk spontaneously. A single investigator, who is masked to the treatment and the animal genotype, performs the neurologic evaluation every 24 h after pMCAO until the animals are euthanized (Mori, et al., J Cereb Blood Flow Metab 25:748-762 [2005]).

To determine brain damage at 1 and 5 days after pMCAO, mice are reanesthetized as described above, and euthanized by transcardial perfusion of 200 mL of 10 U/mL heparin in saline, followed by 200 mL of 4% paraformaldehyde in 0.1 mol/L (pH 7.4) phosphate-buffered saline (PBS). The brain is removed and fixed in the same fixative as above at 4° C. for 48 h. Then, the bilateral cerebral hemispheres are embedded in paraffin with 48 h of processing. Serial sections (5-um in thickness) of the cerebral hemispheres at six predetermined coronal planes separated by 1-mm intervals are sequentially labeled as sections 1 to 6 and stained with hematoxylin and eosin (H&E) or cresyl violet. The infarct area in each section is measured using a computer-based image analyzer (Scion Image beta 4.02 for Windows, Scion Corporation, Frederic, Md., USA). To exclude the effects of brain edema, the infarct area is corrected by the ratio of the whole area of the ipsilateral hemisphere to that of the contralateral hemisphere. Since the interval between sections is 1 mm, the infarct volume (mm3) is calculated as the running sum of corrected infarct area in all six slices (Mori, et al., J Cereb Blood Flow Metab 25:748-762 [2005]). Measurements are performed in a masked manner by a single investigator.

Additional sections adjacent to the coronal brain slice at the level of the anterior commissure (section No. 3) are used for immunohistochemistry. Detection of S100 and GFAP is performed according to the manufacturer's protocol using a Vectastain ABC Elite kit (Vector Laboratories, Burlingame, Calif., USA), coupled with the diaminobenzidine reaction. Rabbit polyclonal anti-S100 and anti-GFAP antibodies (ready to use and diluted 1:1000, respectively; incubated at 4° C. overnight, DAKO, Carpinteria, Calif., USA) are used as primary antibodies; hence, the designation of ‘S100’ is used to describe the corresponding results. Phosphate-buffered saline (0.1 mol/L (pH 7.4)) or normal rabbit serum (isotype control) is used instead of primary antibody or ABC reagent as a negative control (Mori, et al., J Cereb Blood Flow Metab 25:748-762 [2005]).

Images are acquired using an Olympus BX60 microscope with an attached digital camera system (DP-50, Olympus, Tokyo, Japan), and the digital image is routed into a Windows PC for quantitative analysis using SimplePCI software (Compix, Inc. Imaging Systems, Cranberry Township, Pa., USA).

### Example 30: Effect of Anti-ApoE4 Antibodies on Alzheimer's Disease

Genetic variations in ApoE also are associated with Alzheimer's disease (AD) type 2 (Ann Neurol (2009) 65:623-625; Neuron (2009) 63:287-303), a late-onset neurodegenerative disorder characterized by progressive dementia, loss of cognitive abilities, and deposition of fibrillar amyloid proteins as intraneuronal neurofibrillary tangles, extracellular amyloid plaques and vascular amyloid deposits. The major constituent of these plaques is the neurotoxic amyloid-beta-APP 40-42 peptide(s), derived proteolytically from the transmembrane precursor protein APP by sequential secretase processing. The cytotoxic C-terminal fragments (CTFs) and the caspase-cleaved products such as C31 derived from APP are also implicated in neuronal death. Risk for AD increased from 20% to 90% and mean age at onset decreased from 84 to 68 years with increasing number of ApoE4 alleles, as observed in 42 families with late onset AD. Thus ApoE4 gene dose appears to be a major risk factor for late onset AD. In contrast, the ApoE2 allele is associated with a lower risk. The mechanism by which ApoE4 participates in pathogenesis is not known.

Antibodies of the present disclosure are evaluated for their ability to improve and/or slow the progression of Alzheimer's disease and/or forms of dementia, such as vascular dementia or frontotemporal dementia, in subjects, such as for example ApoE4 carrier subjects. Such evaluations can be undertaken in a variety of assays, including for example, in a transgenic animal that carries a human ApoE4 allele, or animals that are transduced otherwise with a vector (e.g., retroviral vector) encoding ApoE4, or which are treated with ApoE4 protein. The animals should also display AD features pathologically and/or clinically (Kim, et al., J Neurosci 31:18007-12 [2011]). Animal breeding uses standard techniques. To analyze these mice, cortical tissues are gently lysed in PBS and modified RIPA (1% NP-40, 1% sodium deoxycholate, 25 mM Tris-HCl, 150 mM NaCl) in the presence of 1× protease inhibitor mixture (Roche). Tissue homogenates are centrifuged at 18,000 relative centrifugal force (rcf) for 30 min. Equal amounts of protein for each sample are run on 4-12% Bis-Tris XT gels (Bio-Rad) and transferred to PVDF membranes. Blots are probed with the following antibodies: ApoE (Academy Biomedical); APP (ZYMED); PS1-NTF (EMD Chemicals); β-secretase 1 (BACE1) (Cell Signaling Technology); synaptophysin (or SYP) (Sigma); glutamate receptor (GluR) 2/3/4 (Cell Signaling Technology); NMDAR2b (Cell Signaling Technology); postsynaptic density protein 95 (PSD-95) (Millipore); and tubulin (Sigma). Tubulin-normalized band intensity is quantified using NIH ImageJ software.

Cortical tissues are sequentially homogenized with PBS, modified RIPA, and 5 M guanidine HCl buffer. Tissue homogenates are centrifuged at 18,000 rcf for 30 min after each extraction. The levels of Amyloidβ and ApoE are measured by enzyme-linked immunosorbent assay (ELISA). For Amyloid β ELISA, HJ2 (anti-Aβ35-40) and HJ7.4 (anti-Aβ37-42) are used as capture antibodies, and HJ5.1-biotin (anti-Aβ13-28) as the detection antibody. Commercial reagent anti-ApoE monoclonal antibodies (e.g., WUE4, Calbiochem) are used for ApoE ELISA (Kim, et al., J Neurosci 31:18007-12 [2011]).

Histology, staining, immunohistochemistry, and quantitative analysis are performed as published and known in the art, except that biotinylated mouse monoclonal antibody HJ3.4 (1:1000, targeted against amino acids 1-13 of the human Aβ sequence) is used to detect Amyloid β in tissue sections. For histology (Kim, et al., J Exp Med 209:2149-56 [2012]) and quantitative analysis of Amyloid β plaques, brain hemispheres are placed in 30% sucrose before freezing and cutting on a freezing sliding microtome. Serial coronal sections at 50-μm intervals are collected from the rostral anterior commissure to caudal hippocampus. Sections are stained with biotinylated 82E1 (anti-Aβ1-16) antibody (1:500 dilution; IBL International) or X-34 dye. Stained brain sections are scanned with a NanoZoomer slide scanner (Hamamatsu Photonics) at 20° magnification setting. For quantitative analyses of 82E1-biotin and X-34 staining, scanned images are exported using NDP viewer software (Hamamatsu Photonics) and converted to 8-bit grayscale using ACDSee Pro 2 software (ACD Systems). All converted images are uniformly thresholded to highlight plaques, and then analyzed by “Analyze Particles” function in the ImageJ software (National Institutes of Health). Identified objects after thresholding are individually inspected to confirm the object as a plaque or not. Three brain sections per mouse, each separated by 300 μm, are used for quantification. These sections correspond approximately to sections at Bregma −1.7, −2.0, and −2.3 mm in the mouse brain atlas. The mean of three sections is used to represent a plaque load for each mouse. For analysis of Aβ plaque in the cortex, the cortex immediately dorsal to the hippocampus is assessed. All analyses are performed in a blinded manner.

Brain sections cut with a freezing sliding microtome are immunostained with anti-CD45 antibody (1:500 dilution; AbD Serotec). Stained brain sections are scanned with a NanoZoomer slide scanner (Hamamatsu Photonics) at 40° magnification setting. The percent area covered by CD45 staining is analyzed in the cortex by using NDP viewer, ACDSee Pro 2, and ImageJ softwares, as described in the previous section. Three brain sections per mouse, each separated by 300 μm, are used for quantification. The mean of three sections is used to estimate the area covered by immunoreactivity. All analyses are performed in a blinded fashion after stained images are thresholded to minimize false-positive signals.

Nine-month-old male APPswe/PS1deltaE9 mice are intraperitoneally injected 4 times every 3 d, and brain tissues are collected 24 h after the last injection. Cerebral cortical tissues are lysed by sonication (3-s pulse, 5 times, 35% amplitude) with lysis buffer (50 mM Tris-HCL, 2 mM EDTA, 1 μg/ml leupeptin, 1 μg/ml aprotinin, 0.25 mM phenylmethanesulfonyl fluoride, pH 7.4). Homogenates are centrifuged for 10 min at 14,000 RPM. Supernatants are used to measure IFN-γ and IL-1α levels using Rodent Cytokine Multi-Analyte Profile (Myriad RBM).

Cortical and hippocampal tissues are sequentially homogenized with PBS and 5 M guanidine buffer in the presence of 1× protease inhibitor mixture (Roche). The levels of insoluble Aβ in a 5-M guanidine fraction are measured by sandwich ELISA. For Aβ ELISA, HJ2 (anti-Aβ35-40) and HJ7.4 (anti-Aβ37-42) are used as capture antibodies, and HJ5.1-biotin (anti-Aβ13-28) is used as the detection antibody. ApoE levels in the plasma and cortical tissue PBS lysates are measured using apoE ELISA. HJ6.2 and HJ6.3 antibodies are used for capture and detection, respectively. Pooled C57BL/6J plasma is used as a standard for murine apoE quantification (Kim, et al., J Exp Med 209:2149-56 [2012]).

### Example 31: Effect of Anti-ApoE4 Antibodies on ApoE4 Dependent Decreases in Brain Volume in ApoE4 Carriers

Antibodies of the present disclosure are evaluated for their ability to inhibit and/or slow the progression of ApoE4 dependent decreases in brain volume in ApoE4 carrier subjects. Volumetric imaging of human brain from ApoE4 carrier subjects has shown a progressive reduction (Driscoll, et al., Neurology 72:1906-13 [2009]). The effect of anti-ApoE4 antibody is determined in pathological volumetric analysis of brain regions, such as for example, hippocampus CA1, entorhinal cortex, or subiculum, in knockin ApoE4 animals, and for example, with or without AD associated transgenes, as detailed above and elsewhere (McDaniel, et al., Neuroimage 14:1244-55 [2001]).

### Example 32: Effect of Anti-ApoE4 Antibodies on Cognitive Deficit in ApoE4 Carriers

Antibodies of the present disclosure are evaluated for their ability to inhibit and/or slow the progression of cognitive deficit. Mice are group housed with littermates in the breeding room (12-h light:12-h dark cycle, lights on 07:00-19:00; food and tap water available ad libitum). All experimental mice are fed a Normal Diet formulation (crude proteins 22%, crude fat 4.3%, crude fiber 4% and ash 5.5%; A03 from UAR France). At 15 months of age, mice are weighed and housed individually on day 1 of the testing schedule. One week later, mice are daily weighed and handled for 2-3 min (days 6-11). Then, mice are successively tested in a spatial recognition task in an open field, in a spatial reference memory task, a spatial DMP task and a visible platform task in a water-maze, a Y-maze active avoidance task, a step-through passive avoidance task and a footshock threshold determination (Bour, et al., Behav Brain Res 193:174-82 [2008]). The sequence of behavioral tasks follows the principle of testing from the least to the most invasive, and from the most to the least sensitive to prior test history. This battery of tasks has been successfully pre-tested on C57BL/6J and apoE−/− male mice before being applied to a cohort similar in origin, housing, genetic background, sex and group size, as described (Bour, et al., Behav Brain Res 193:174-82 [2008]).

The investigator is blind to the genotype of the mice under examination throughout the testing period. In between the tasks, mice are left undisturbed for 1-2 weeks. In order to minimize sex-related effects of recent olfactory traces on behavior, male and female mice are tested separately, 4 weeks apart, with the testing devices being cleaned thoroughly with alcohol between male and female series. Mice are weighed after completion of testing, on day 81. Weight of day 81 minus weight of day 1 (Wt81−Wt1) is calculated for each mouse to evaluate the weight evolution over the testing period.

The spatial recognition task is based on the spontaneous tendency of mice to explore preferentially displaced versus non-displaced objects from a familiar arrangement of objects. The apparatus consists of a Plexiglas open field (52 cm×52 cm) with black walls (40 cm high) and a translucent floor divided into 25 equal squares by black lines. The floor is dimly illuminated by a 60 W bulb, placed 32 cm centrally underneath. At the mouse level, it results in 50 lx (corners) to 100 lx (center) light intensity. A black and white striped card (21 cm×29.7 cm) is fixed against one wall. Five objects different in shape (size ranging from 2.4 to 3.8 cm), color and material (a glass black marble, a porcelain thimble, a gray plastic toothed wheel, a white plastic rod on a blue rectangular counter, and a red plastic half gear wheel) are used. Mice are submitted to three exploration phases separated by 5-min resting periods in their home cage. The task begins with a 5-min habituation period in an empty open-field, a 15-min acquisition phase in presence of an arrangement of five different objects, and a 15-min retention phase with a new arrangement of an identical set of object, two of them being relocated. Thus, two categories of objects are considered, the displaced objects (marble and gray wheel) and the non-displaced objects (thimble, rod on counter and red wheel). Object exploration is defined as the mouse nose pointing to the object at a distance less than 1 cm. The amount of time spent exploring each category of objects is recorded with stopwatches over the 15-min phases. These values are divided by the number of objects for each category in order to obtain the mean exploration time for displaced objects (D) and non-displaced objects (ND). Spatial recognition performance is analyzed in terms of a spatial recognition index for two reasons: (i) as a ratio of exploration duration: D/(D+ND), it is independent from the duration of exploration which might differ among groups and (ii) it is also adopted in previous studies (Bour, et al., Behav Brain Res 193:174-82 [2008]).

Total exploration over the two categories of objects (2D+3ND) provides an indication on the mice reaction towards the whole set of objects during each phase. Locomotor activity is evaluated in terms of distance traveled (expressed in cm/5 min period for each phase) by means of a videotracking system (Ethovision 2.3, Noldus Information Technology, The Netherlands). It is verified that the two categories of objects, D and ND, are equally explored by the mice during the acquisition session in order to avoid a bias due to spontaneous preference for an object or a set of objects (Bour, et al., Behav Brain Res 193:174-82 [2008]).

Mice are weighed daily before the first trial in the water-maze (diameter: 140 cm; platform size: 10 cm; water temperature: 19±1° C.). The water is made opaque by the addition of milk powder and the milky water is changed daily. All tasks consist in finding a platform to escape from the water. Trajectories are recorded and analyzed with the videotracking system. When the mouse does not find the platform, it is gently guided and allowed to stay on it for 10 s. Once the mouse voluntarily climbs on a transporting grid, it is placed in its home cage under a red heating lamp to prevent hypothermia. During the first week, mice are trained in the spatial reference memory protocol. They first receive a water adaptation trial (a 1-min walk in 2-cm deep water with a visible platform) on day 21, followed by a 120-s free swim trial (no platform present) on day 22. Spatial reference memory training per se begins on day 25. Mice receive four trials a day for 4 consecutive days with the submerged platform always on the same location (center of the west virtual quadrant). Each trail starts from one of four possible start positions, the sequence of which varies daily. Three mice are tested within a 20-30-min session, which results in an inter-trial-interval (ITI) of 5-10 min. The day following the end of reference memory training (day 29), mice are subjected to a probe trial (60 s, no platform) in order to examine their long-term spatial memory performance.

After a 2-day resting period, mice are trained on a DMP protocol using the same water-maze. From day 32 to 35, the position of the submerged platform varies daily from one quadrant center to the other (sequence: east, north, south and west quadrants). Mice start from one of two possible starting points, both opposite and equidistant to the platform's position. Again, training consists of four trials a day for 4 consecutive days. An ITI of 1 h is set between trials 1 and 2 and then 5-min ITIs between trials 2-3 and 3-4. This protocol is used to determine retention memory after a 1-h delay between trials 1 and 2. The remaining trials with short ITIs allows all groups to reach a common level of performance by the end of each daily session. The day after completion of the DMP task, mice are tested for their visual/motivational abilities in a visible platform task. For each of the four trials, the position of the visible platform (1 cm above the water surface) changes from one quadrant center to the other. The start position changes as well, but remains at the same distance from the platform. All trials are recorded and analyzed with the videotracking system.

Two weeks after the last water-maze trial, mice are subjected to the Y-maze avoidance learning task, which involves procedural memory with a place discrimination component and a temporal component. The procedural aspect of this task lies in the need of a large number of trials to learn a specific motor response (go to the left alley within 5 s) and the existence of a spontaneous improvement of performance (also called “off-line” improvement) on the temporal component of the task. This improvement, which develops several hours after initial training in C57BL/6J and other mouse strains, is known to be extremely sensitive to amnestic.

Mice are trained in a transparent Plexiglas apparatus with three identical arms in a Y shape. At the end of each alley (13 cm×4.5 cm×5.5 cm) is a mobile box (10 cm×4.5 cm×5.5 cm), which allows for transport of the mouse from the goal alley to the start position without having to handle it. In each trial, the mouse has to leave the start-alley of the maze within 5 s (temporal component) and has to choose the left alley (discrimination component) to avoid footshocks. Therefore, a mouse can make two types of errors within a trial: an active avoidance error when it fails to leave the start alley within 5 s, and/or a discrimination error when it choses the wrong alley. Footshocks are delivered every 7 s until the mouse enters the right alley. The footshock level is individually set (maximum 40 V, ac) over the first trial or two in such a way that the mouse lifts suddenly one or two paws from the grid. The mouse undergoes one trial every minute until it reached a criterion of seven correct out of eight consecutive trials. Retention memory performance is tested 48 h later with the same criterion and the same individually set footshock level. Avoidance errors and discrimination errors are recorded in order to evaluate the mouse performance on both the temporal component and the discrimination component of the task, respectively.

One week after the Y-maze task, mice are tested in a step-through passive avoidance task. The apparatus consists of a light, white compartment (8 cm width×23 cm long×14 cm high) and a dark, black compartment (8 cm width×15 cm long×14 cm high) separated by a guillotine door. During the acquisition trial, the mouse is placed in the light compartment. The door is opened 1 min later. The time to enter the dark compartment is recorded. Once all four paws are in the dark compartment, the door is closed and the mouse immediately received two footshocks (40 V, ac; 0.3 s duration; 5 s apart). After 15 s, the mouse is removed from the dark compartment, and returned to its home cage. The mouse is placed back into the light compartment 24 h later. After 10 s, the door is opened and the following measures are taken over 10 min: (1) latency to enter the dark compartment; (2) number of black/white compartment transitions; (3) total time spent in the black compartment. The mouse is always placed against the wall opposite to the dark compartment, so it has to cross the white compartment to reach the guillotine door. Approach behavior towards the dark compartment is evaluated through the latency to cross the white compartment (all four paws in the second half of this compartment).

One week after the passive avoidance task, the threshold for footshock sensitivity is determined with the same apparatus used in the passive avoidance paradigm. This test is conducted to verify that different mouse lines have similar footshock sensitivity threshold. The mouse is placed in a long black alley (8 cm width×50 cm long×14 cm high). The level of footshocks is progressively increased (2 V intervals) starting at 16 V with a maximum of 40 V. Mice receive shocks of increasing voltage every 15-45 s until a footshock induces a flight response. This level of footshock is considered as the threshold of the mouse (Bour, et al., Behav Brain Res 193:174-82 [2008]).

What is claimed is:

1. An isolated antibody that specifically binds to a lipidated ApoE4 protein.

2. The antibody of claim 1, wherein the antibody binds to one or more amino acid residues within an ApoE4 epitope selected from:

| | |
|---|---|
| (a) | amino acid residues 55-78 |
| | (QVTQELRALMDETMKELKAYKSEL (i.e., SEQ ID NO: 2)) |
| | of SEQ ID NO: 1; |
| (b) | amino acid residues 134-150 |
| | (RVRLASHLRKLRKRLLR (i.e., SEQ ID NO: 3)) |
| | of SEQ ID NO: 1; |
| (c) | amino acid residues 154-158 |
| | (DLQKR (i.e., SEQ ID NO: 4)) of SEQ ID NO: 1; |
| (d) | amino acid residues 208-272 |
| | (QAWGERLRARMEEMGSRTRDRLDEVKEQVAEVRAKLEEQAQQIRLQ |
| | AEAFQARLKSWFEPLVEDM (i.e., SEQ ID NO: 5)) |
| | of SEQ ID NO: 1; |
| (e) | amino acid residues 225-299 |
| | (TRDRLDEVKEQVAEVRAKLEEQAQQIRLQAEAFQARLKSWFEPLVE |
| | DMQRQWAGLVEKVQAAVGTSAAPVPSDNH (i.e., SEQ ID NO: |
| | 6)) of SEQ ID NO: 1; and |
| (f) | amino acid residues 244-272 |
| | (EEQAQQIRLQAEAFQARLKSWFEPLVEDM (i.e., SEQ ID |
| | NO: 7)) of SEQ ID NO: 1. |

3. The antibody of claim 1, wherein the antibody binds to an ApoE4 epitope comprising at least one of amino acid residues Arg-61, Glu-109, Arg-112, Arg-136, His-140, Lys-143, Arg-150, Asp-154, Arg-158, Arg-172, and Glu-255 of SEQ ID NO: 1.

4. The antibody of claim 1, wherein the antibody disrupts the interaction between an N-terminal domain and C-terminal domain of an ApoE4 protein.

5. The antibody of claim 4, wherein the antibody disrupts the interaction between ApoE4 helix 2, comprising amino acid residues 55-78 (QVTQELRALMDETMKELKAYKSEL (i.e., SEQ ID NO: 2)) of SEQ ID NO: 1, and the ApoE4 lipid binding domain, comprising amino acid residues 244-272 (EEQAQQIRLQAEAFQARLKSWFEPLVEDM (i.e., SEQ ID NO: 7)) of SEQ ID NO: 1.

6. The antibody of claim 4, wherein the antibody disrupts the interaction between amino acid residues Arg-61 and Glu-255 of SEQ ID NO: 1.

7. The antibody of claim 1, wherein the antibody has one or more activities, in vitro or in a subject, selected from:
(a) increasing binding of lipidated ApoE4 to a phospholipid-rich particle;
(b) reducing binding of lipidated ApoE4 to a triglyceride rich lipid particle;
(c) increasing the release of ApoE4 from a triglyceride-rich lipid particle;
(d) reducing the binding of lipidated ApoE4 to LDLR;
(e) reducing the binding of lipidated ApoE4 to an LDLR family member;
(f) increasing binding of ApoE4 to HSPG;
(g) reducing ApoE4-associated processing of APP to amyloid beta;
(h) reducing ApoE4-associated inhibition of amyloid beta clearance;
(i) reducing ApoE4-associated BBB leakage;
(j) reduces ApoE4-associated formation of neurofibrillary tangles;
(k) reducing ApoE4-associated inflammation;
(l) reducing ApoE4-associated production of amyloid beta;
(m) reducing ApoE4-associated reduction in clearance of amyloid beta across the BBB, or increasing clearance of amyloid beta across the BBB;
(n) reducing ApoE4-associated accumulation of amyloid beta in tissue, or increasing clearance of amyloid beta from a tissue;
(o) reducing ApoE4-associated intraneuronal accumulation of amyloid beta;
(p) reducing ApoE4-associated internalization of amyloid beta into nerve cells;
(q) reducing ApoE4-associated stabilization of amyloid beta and the formation of amyloid beta multimers;
(r) reducing ApoE4-associated increase in LDL cholesterol levels;
(s) reducing ApoE4-associated clinically undesirable lipid profiles;
(t) reducing ApoE4-associated downregulation of LDLR on cell surfaces;
(u) reducing ApoE4-associated downregulation of LDLR protein family members on cell surfaces;
(v) reducing ApoE4-associated delayed recovery from traumatic or non-traumatic acquired brain injury;
(w) reducing ApoE4-associated risk of developing Alzheimer's disease or late onset Alzheimer's disease, or symptoms or pathology thereof;
(x) reducing ApoE4-associated risk of developing cardiovascular disease or symptoms or pathology thereof;
(y) reducing ApoE4-associated risk of developing dementia or symptoms or pathology thereof;
(z) reducing ApoE4-associated risk of developing cerebral amyloid angiopathy or symptoms or pathology thereof;
(aa) reducing ApoE4-associated risk of developing multiple sclerosis or symptoms or pathology thereof;
(bb) reducing ApoE4-associated risk of developing age-related macular degeneration or symptoms or pathology thereof;
(cc) reducing ApoE4-associated acceleration of aging;
(dd) reducing or delaying ApoE4-associated cognitive impairment, or normalizing cognitive function in a subject expressing ApoE4;
(ee) reducing ApoE4-associated inhibition of phagocytosis in microglia, macrophages, monocytes, or astrocytes;
(ff) reducing ApoE4-associated decrease in soluble amyloid beta uptake by astrocytes;
(gg) reducing ApoE4-associated depletion of myelin cholesterol;
(hh) reducing ApoE4-associated adverse drug reaction to statin therapy or poor responsiveness to statin therapy;
(ii) reducing ApoE4-associated aberrant gene expression profiles associated with Alzheimer's disease;
(jj) reducing ApoE4-associated reduction in glucose metabolism in brains of pre-symptomatic Alzheimer's disease patients;
(kk) reducing ApoE4-associated reduction in volume of brain structures in pre-symptomatic Alzheimer's disease patients;
(ll) reducing ApoE4-associated senile plaque formation;
(mm) reducing ApoE4-associated decrease in amyloid beta uptake by neurons, astroglia, microglia, oligodendroglia or endothelial cells;
(nn) reducing ApoE4-associated pathological microglial activity;
(oo) reducing the binding of ApoE4 to LRP1, thereby decreasing ApoE4's ability to compete with soluble amyloid beta for binding to LRP1;
(pp) reducing ApoE4-associated reduction in clearance of apoptotic neurons, nerve tissue debris, non-nerve tissue debris, bacteria, foreign bodies, or disease-associated proteins or peptides;
(qq) and combinations thereof.

8. The antibody of claim 7, wherein the phospholipid-rich particle is an HDL particle.

9. The antibody of claim 7, wherein the triglyceride-rich particle is a VLDL particle.

10. The antibody of claim 7, wherein the LDLR family member is selected from LDLR, VLDLR, LRP1, LRP1b, LRP2, LRP3, LRP4, LRP5, LRP6, LRP7, LRP8, LRP10, LRP11, LRP12 sortilin, TREM2, and combinations thereof.

11. The antibody of claim 1, wherein ApoE4 binding to atypical LDLR family members is preserved in the presence of the antibody and wherein the atypical LDLR family member is selected from TREM2, sortilin, SORL1, SORCS1, SORCS2, SORCS, and combinations thereof.

12. The antibody of claim 1, wherein the antibody is a monoclonal antibody.

13. The antibody of claim 1, wherein the antibody is an antibody fragment.

14. A method of preventing, treating or reducing the risk of a disease, condition or disorder in a subject that is an ApoE4 carrier, comprising administering to the subject a therapeutically effective amount of an isolated antibody that specifically binds to a lipidated ApoE4 protein.

15. The method of claim 14, wherein the disease, condition or disorder is selected from the group consisting of dementia, cognitive disorder, Alzheimer's disease, cerebral amyloid angiopathy, cardiovascular disease, age-related macular degeneration, multiple sclerosis, traumatic or non-traumatic acquired brain injury, adverse reaction or poor responsiveness to statin therapy, reduced glucose metabolism in the brain, reduced volume of brain structures, hypercholesterimia, lipoprotein glomerulopathy, sea-blue histiocyte disease, and combinations thereof.

16. The method of claim 15, wherein the subject has a genotype selected from:
(a) an ε4 homozygote;
(b) an ε4/ε3 heterozygote; and
(c) an ε4/ε2 heterozygote.

17. The method of claim 15, further comprising administering to the subject a therapeutically effective amount of one or more additional therapeutic agents selected from an amyloid beta-directed therapeutic, a tau protein-directed therapeutic, an antibody that binds a CD33 protein, an antibody that binds a sortilin protein, an antibody that binds a TREM2 protein, an antibody that binds an amyloid beta protein, an antibody that binds tau protein, a BACE inhibitor, a gamma secretase inhibitor, an agent that disaggregates amyloid beta oligomers, an agent that disaggregates tau fibrils, and combinations thereof.

18. A method of increasing binding of lipidated ApoE4 to a phospholipid-rich particle and decreasing binding of lipidated ApoE4 to a triglyceride rich lipid particle, comprising contacting the lipidated ApoE4 with an isolated antibody that specifically binds to a lipidated ApoE4 protein.

19. The method of claim 18, wherein the contacting is performed in vitro.

20. The method of claim 18, wherein the contacting is performed in a subject.


SEQUENCE LISTING (7 sequences)

SEQ ID NO: 1 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 2 - protein, 24 aa, Homo sapiens
QVTQELRALMDETMKELKAYKSEL

SEQ ID NO: 3 - protein, 17 aa, Homo sapiens
RVRLASHLRKLRKRLLR

SEQ ID NO: 4 - protein, 5 aa, Homo sapiens
DLQKR

SEQ ID NO: 5 - protein, 65 aa, Homo sapiens
QAWGERLRARMEEMGSRTRDRLDEVKEQVAEVRAKLEEQAQQIRLQAEAFQARLKSWFEPLVEDM

SEQ ID NO: 6 - protein, 75 aa, Homo sapiens
TRDRLDEVKEQVAEVRAKLEEQAQQIRLQAEAFQARLKSWFEPLVEDMQRQWAGLVEKVQAAVGTSAAPVPSDNH

SEQ ID NO: 7 - protein, 29 aa, Homo sapiens
EEQAQQIRLQAEAFQARLKSWFEPLVEDM